(19) United States
(12) Patent Application Publication
(10) Pub. No.: US 2023/0277538 A1
(43) Pub. Date: Sep. 7, 2023

(54) THERAPEUTICS FOR THE TREATMENT OF FSHD

(71) Applicant: UCL BUSINESS LTD, London (GB)

(72) Inventors: Julie DUMONCEAUX, London (GB); Virginie MARIOT, London (GB); Maximilien BENCZE, London (GB)

(21) Appl. No.: 18/011,583

(22) PCT Filed: Jun. 23, 2021
(86) PCT No.: PCT/GB2021/051600

§ 371 Date: Dec. 20, 2022

(30) Foreign Application Priority Data
Jun. 24, 2020 (GB) 2009618.6

(51) Int. Cl. A61K 31/506; C12N 15/86; A61K 31/502; A61P 21/00

(52) U.S. Cl. CPC A61K 31/506; C12N 15/86; A61K 31/502; A61P 21/00; C12N 2750/14141

(57) ABSTRACT

The present invention relates to methods for treating facioscapulohumeral dystrophy (FSHD). Specifically, the invention relates to the use of an inhibitor of necroptosis for treating FSHD.

### FIELD OF THE INVENTION

The present invention relates to methods for treating facioscapulohumeral dystrophy (FSHD). Specifically, the invention relates to the use of an inhibitor of necroptosis for treating FSHD.

### BACKGROUND OF THE INVENTION

Muscular dystrophies (MDs) are a group of genetic diseases. The group is characterized by progressive weakness and degeneration of the skeletal muscles that control movement. Some forms of MD develop in infancy or childhood, while others may not appear until middle age or later. The disorders differ in terms of the distribution and extent of muscle weakness (some forms of MD also affect cardiac muscle), the age of onset, the rate of progression, and the pattern of inheritance.

Facioscapulohumeral muscular dystrophy (FSHD) is a complex autosomal dominant disorder characterized by progressive and asymmetric weakness of facial, shoulder and limb muscles. Symptoms typically arise in adulthood with most patients showing clinical features before age thirty. About five percent of patients develop symptoms as infants or juveniles and these are generally more severely affected. Clinical presentation can vary from mild (some limited muscle weakness) to severe (wheelchair dependence). Historically, FSHD was classified as the third most common MD, affecting one in 20,000 individuals worldwide. However, recent data indicate FSHD is the most common MD in Europe, suggesting its worldwide incidence may be underestimated.

FSHD is characterized by a loss of repressive epigenetic marks within the D4Z4 macrosatellite located in the sub-telomeric region of chromosome 4 (van Deutekom, 1993) (Wijmenga, 1992) (Lemmers, 2012). In muscle, chromatin relaxation results in the expression of the DUX4 transcription factor whose ORF is present in each D4Z4 repeat, resulting in a poison protein effect through induction of multiple downstream genes (Snider, 2009) (Ferreboeuf, 2014). DUX4 expression is extremely low but it has been robustly found in adult and fetal FSHD muscle cells and biopsies (Snider, 2010) (Broucqsault, 2013) (Ferreboeuf, 2014). DUX4 was proposed to disrupt multiple cellular functions (for review see DeSimone, 2017) and to induce cell death in different models. Several publications have reported DUX4-mediated cell death in vitro in murine C2C12 or human myotubes (Bosnakovski, 2008) (Rickard, 2015) (Block, 2013) and in vivo in different species including mice (Wallace, 2011) (Dandapat, 2014).

Many of the genes and pathways involved in FSHD have not yet been deciphered. Previously, a possible pro-apoptotic role of DUX4 was proposed, based on a caspase 3/7 activity found after DUX4 overexpression in vitro (Kowaljow, 2007)(Bosnakovski, 2008), the presence of TUNEL-positive nuclei in Xenopus embryos overexpressing DUX4 (Wuebbles, 2010), the activation of a p53-dependent cell death observed after DUX4 over-expression in mouse muscles and p53-knockout mouse background suppressed AAV-DUX4 toxicity (Wallace, 2011), the decrease of caspase 3/7 activation after treatment of FSHD myotubes with p53 pathway inhibitors (Block, 2013) and bio-informatics analysis (Rickard, 2015) (Shadle, 2017) (Lek, 2020). DUX4-expressing cells have been also described to be more susceptible to oxidative stress-induced death (Bosnakovski, 2008) (Winokur, 2003) (Bou Saada, 2016) (Dmitriev, 2016). However, the role of apoptosis and/or oxidative stress-induced death in FSHD has not been confirmed. Further, heterocyclic derivatives that inhibit tumor necrosis factor alpha (TNF-α)-induced necroptosis have been disclosed (WO 2010/075290). However, these inhibitors have not been shown to have utility in treating FSHD.

Uncertainty surrounding the genes and pathways involved in FSHD has resulted in a lack of treatment options. There thus remains a need in the art for a treatment for muscular dystrophies including FSHD.

### SUMMARY OF THE INVENTION

The inventors have identified for the first time that necroptosis plays a key role in the pathology of FSHD.

Described herein, the inventors show that necroptosis contributes to DUX4 mediated toxicity both in vitro and in vivo.

The inventors have shown that DUX4 mediates necroptosis-dependent cell death. In particular, in vitro addition of a Z-vad pan-caspase inhibitor to iC2C12-DUX4 myotubes did not increase cell survival, suggesting a limited role of apoptosis in myotube death. The addition of the GSK'872 RIPK3 inhibitor (in combination with Z-Vad), however, led to a 2-fold increase of the number of viable cells. Thus, the involvement of necroptosis in myotube death was established.

In iC2C12 myoblasts, RIPK1 was shown to be the main regulator of the necroptosis pathway. Specifically, the presence of RIPK1 inhibitor Necrostatin-1 caused a 20% increase in the number of viable cells. RIPK3 is also involved since the presence of RIPK3 inhibitor GSK'872 caused a 10% increase in the number of viable cells. Apoptosis did not participate in cell death and the addition Z-Vad did not modify the percentage of viable cells.

In vivo, the inventors created a new transgenic and viable mouse model lacking RIPK3 and expressing DUX4 only after tamoxifen injection. RIPK3 was targeted because RIPK1-deficient mice show perinatal lethality (Kelliher, 1998). In these mice, it was observed that RIPK3 deficiency reduces the weight loss observed after DUX4 expression.

These studies thus provide evidence for a key role of necroptosis and RIPK3 in DUX4-mediated cell death and provide proof-of-principle for treating FSHD using one or more inhibitors of the necroptosis pathway, optionally wherein the inhibitor is an inhibitor of RIPK3, RIPK1 or MLKL.

To target the necroptosis pathway, the inventors have developed a shRNA gene silencing approach. It is envisaged that shRNA targeting components of the necroptosis pathway can be used as a treatment for FSHD in human subjects. In particular, it is envisaged that adeno-associated viral (AAV) vector-delivered therapeutic shRNA (or other antisense oligonucleotides) targeting expression of the components of the necroptosis pathway can be used to treat FSHD in humans.

The present invention therefore provides an inhibitor of the necroptosis pathway for use in a method of treating facioscapulohumeral dystrophy (FSHD).

The inhibitor may be administered to a patient having aberrant activation of necroptosis pathway in muscle tissue. The inhibitor may inhibit the expression or activity of a protein involved in the necroptosis pathway. In particular, the inhibitor may inhibit the gene expression of at least one of RIPK3, RIPK1 or MLKL, or inhibit the protein activity of at least one of RIPK3, RIPK1 or MLKL. For example, the inhibitor may inhibit the expression of least one of human RIPK3, human RIPK1 or human MLKL. Alternatively, the inhibitor may inhibit the activity of at least one of human RIPK3, human RIPK1 or human MLKL. Inhibition may result in an at least 80% reduction in expression of the target gene or an at least 80% reduction in the activity of the target protein. Preferably, the inhibitor is administered to a target cell of muscular lineage, such as a myoblast, or a myotube, or a mature myofibre.

Administration of the inhibitor improves or alleviates one or more symptoms of FSHD, including muscle atrophy, muscle weakness, for example abdominal muscle weakness, hip weakness, lower leg weakness (e.g., peroneal muscle weakness), shoulder weakness (e.g., scapular winging) and/or facial weakness, lordosis, scoliosis, dysphagia, foot drop, inflammation of the muscles, retinal vasculopathy, hearing loss, respiratory involvement or any combination thereof.

Preferably, the inhibitor is an antisense oligonucleotide or a small molecule inhibitor.

Where the inhibitor is a small molecule inhibitor, the inhibitor is preferably selected from a small molecule inhibitor of a component of the necroptosis pathway, such as Ripk3, Ripk1, Mlk1. For example, the inhibitor is preferably selected from dabrafenib, galavit, necrostatin-1, GSK872 or ponatinib.

Where the inhibitor is an antisense oligonucleotide, the antisense oligonucleotide preferably targets an RNA molecule comprising or consisting of any one of SEQ ID NO: 1 to 17, or a variant or derivative thereof. The skilled person will appreciate that suitable variants include transcript variants. The antisense oligonucleotide preferably comprises a sequence that is 100% complementary to a sequence of at least 10, at least 11, at least 12, at least 13, at least 14, at least 15, at least 16, at least 17 or at least 18 contiguous nucleotides of any one of SEQ ID NO: 18 to 35. Preferably, the antisense oligonucleotide comprises a sequence that is 100% complementary to the sequence of any one of SEQ ID NOs: 18 to 35. In a preferred embodiment, the antisense oligonucleotide is an shRNA.

Also provided is an expression construct encoding the antisense oligonucleotide for use in a method of treating facioscapulohumeral dystrophy (FSHD). Preferably, the expression construct comprises a nucleotide sequence encoding an antisense oligonucleotide operably linked to a promoter. Preferred promoters include RNA Pol III promoters or muscle-preferred or muscle-specific promoters. In particular, RNA Pol III promoters may be selected from a U6, H1 or 7SK promoter, or any derivative thereof.

Also provided is a vector comprising the expression construct the invention for use in a method of treating facioscapulohumeral dystrophy (FSHD). The vector may be a viral vector, such as an AAV vector. Preferred serotypes of the AAV vector target muscle tissue, such as AAV 1, 6, 8, 9 or rhesus serotype 74 (rh74).

Also provided is a vector for use in a method of treating facioscapulohumeral dystrophy (FSHD), wherein the vector comprises a sequence encoding an antisense oligonucleotide comprising a sequence that is 100% complementary to an RNA molecule having the sequence of any one of SEQ ID NOs: 18 to 35, said coding sequence being operably linked to an RNA Pol III promoter such that expression of said antisense oligonucleotide in a cell of muscular lineage reduces the expression of at least one of human RIPK3, RIPK1 or MLKL, thereby alleviating at least one symptom of FSHD.

Also provided is a pharmaceutical composition for use in a method of treating FSHD wherein the pharmaceutical composition comprises the small molecule inhibitor, the antisense oligonucleotide, the expression construct, or the vector of the invention, further comprising a pharmaceutically acceptable carrier, diluent, excipient, adjuvant, buffer and/or stabilizer. Preferably, the composition is administered to a human subject.

Also provided is a vector comprising an expression construct encoding an antisense oligonucleotide which targets at least one of RIPK3, RIPK1 or MLKL, wherein the coding sequence is operably linked to a promoter. Preferably, expression of the antisense oligonucleotide silences the gene expression, transcription, translation and/or protein activity of RIPK3, RIPK1 or MLKL. For example, the antisense oligonucleotide may target an RNA molecule having the sequence of any one of SEQ ID NO: 1 to 17. Preferably, the antisense oligonucleotide targets an RNA molecule comprising a sequence of at least 10, at least 11, at least 12, at least 13, at least 14, at least 15, at least 16, at least 17 or at least 18 contiguous nucleotides selected from any one of SEQ ID NO: 18 to 35. Described herein, the vector may be a viral vector, such as an AAV vector. Preferred serotypes of the AAV vector target muscle tissue, such as AAV 1, 6, 8, 9 or rhesus serotype 74 (rh74).

Also provided is a pharmaceutical composition comprising the vector of the invention and a pharmaceutically acceptable carrier, diluent, excipient, adjuvant, buffer and/or stabilizer.

### BRIEF DESCRIPTION OF THE SEQUENCES

SEQ ID NO: 1—mRNA sequence of Homo sapiens receptor interacting serine/threonine kinase 3 (RIPK3) (corresponding to NCBI Reference Sequence: NM_006871.4)
SEQ ID NO: 2—mRNA sequence of Homo sapiens receptor interacting serine/threonine kinase 1 (RIPK1), transcript variant 1 (corresponding to NCBI Reference Sequence: NM_003804.6)
SEQ ID NO: 3—mRNA sequence of Homo sapiens receptor interacting serine/threonine kinase 1 (RIPK1), transcript variant 2 (corresponding to NCBI Reference Sequence: NM_001317061.3)
SEQ ID NO: 4—mRNA sequence of Homo sapiens receptor interacting serine/threonine kinase 1 (RIPK1), transcript variant 3 (corresponding to NCBI Reference Sequence: NM_001354930.2)
SEQ ID NO: 5—mRNA sequence of Homo sapiens receptor interacting serine/threonine kinase 1 (RIPK1), transcript variant 4 (corresponding to NCBI Reference Sequence: NM_001354931.2)
SEQ ID NO: 6—mRNA sequence of Homo sapiens receptor interacting serine/threonine kinase 1 (RIPK1), transcript variant 5 (corresponding to NCBI Reference Sequence: NM_001354932.2)
SEQ ID NO: 7—mRNA sequence of Homo sapiens receptor interacting serine/threonine kinase 1 (RIPK1), transcript variant 6 (corresponding to NCBI Reference Sequence: NM_001354933.2)
SEQ ID NO: 8—mRNA sequence of Homo sapiens receptor interacting serine/threonine kinase 1 (RIPK1), transcript variant 7 (corresponding to NCBI Reference Sequence: NM_001354934.2)
SEQ ID NO: 9—mRNA sequence of Homo sapiens mixed lineage kinase domain like pseudokinase (MLKL), transcript variant 1 (corresponding to NCBI Reference Sequence: NM_152649.4)
SEQ ID NO: 10—mRNA sequence of Homo sapiens mixed lineage kinase domain like pseudokinase (MLKL), transcript variant 2 (corresponding to NCBI Reference Sequence: NM_001142497.3)
SEQ ID NO: 11—mRNA sequence of Mus musculus receptor-interacting serine-threonine kinase 3 (Ripk3), transcript variant 1 (corresponding to NCBI Reference Sequence: NM_019955.2).
SEQ ID NO: 12—mRNA sequence of Mus musculus receptor-interacting serine-threonine kinase 3 (Ripk3), transcript variant 2 (corresponding to NCBI Reference Sequence: NM_001164107.1)
SEQ ID NO: 13—mRNA sequence of Mus musculus receptor-interacting serine-threonine kinase 3 (Ripk3), transcript variant 3 (corresponding to NCBI Reference Sequence: NM_001164108.1)
SEQ ID NO: 14—mRNA sequence of Mus musculus receptor (TNFRSF)-interacting serine-threonine kinase 1 (Ripk1), transcript variant 1 (corresponding to NCBI Reference Sequence: NM_009068.3)
SEQ ID NO: 15—mRNA sequence of Mus musculus receptor (TNFRSF)-interacting serine-threonine kinase 1 (Ripk1), transcript variant 2 (corresponding to NCBI Reference Sequence: NM_001359997.1)
SEQ ID NO: 16—mRNA sequence of Mus musculus mixed lineage kinase domain-like (Mlk1), transcript variant 1 (corresponding to NCBI Reference Sequence: NM_001310613.1)
SEQ ID NO: 17—mRNA sequence of Mus musculus mixed lineage kinase domain-like (Mlk1), transcript variant 2 (corresponding to NCBI Reference Sequence: NM_029005.3)
SEQ ID NOs: 18 to 35—Exemplary target mRNA sequences
SEQ ID NOs: 36 to 59—RT-PCT forwards and reverse primers
SEQ ID NO: 60—Exemplary expression construct (5′ to 3′) encoding Ripk3 shRNAs operably linked to pol III promoters.
SEQ ID NO: 61: Expression construct (5′ to 3′) used in Merienne, 2017

### BRIEF DESCRIPTION OF THE FIGURES

FIG. 1: DUX4 expression triggers cell death in iC2C12-DUX4 myoblasts. (A, B, D, E, F) expression of DUX4, Tm7sf4, Ripk1, Mlk1, or Ripk3 respectively (RT-qPCR) 1 days after addition different concentration of dox in iC2C12-DUX4 myoblasts. (C) Cell viability assay (adenosine triphosphate (ATP) assay) after doxycycline induction at various concentrations. Data represents the mean±SD on 3 independent experiments. *P<0.05, **P<0.01, ***P<0.001

FIG. 2: Necroptosis participates in DUX4-mediated cell death. (A) Cell viability of iC2C12-DUX4 myoblasts at 48 h of induction without or with dox (200 ng/ml) and treatment with various concentrations of the pan-caspase inhibitor Z-VAD (μM). (B) ATP assay on iC2C12-DUX4 myoblasts at 48 h of induction with 100 or 200 ng/ml of dox and treatment with different molecules(Z: ZVAD 2004, N: necrostatin-1 3004; G: GSK'872 204; C: cyclosporin A 0.2 μM) alone or in combination. Ctrl: DMSO alone.

FIG. 3: DUX4 expression triggers cell death in iC2C12-DUX4 myotubes. (A, B, D E, F) Expression of DUX4, Tm7sf4, Ripk1, Mlk1, or Ripk3 respectively (RT-qPCR, relative to Gapdh) was measured 24 hours after addition different concentration of dox in iC2C12-DUX4 myotubes after 4 days of differentiation. (C) Cell viability assay (adenosine triphosphate (ATP) assay) at 24 hours after dox induction at various concentrations (ng/ml).

FIG. 4: Ripk3 inhibition decreases DUX4-mediated cell death in iC2C12-DUX4 myotubes. (A) iC2C12-DUX4 viability in presence of dox 1000 ng/ml and treated with different doses of Z-VAD, Necrostatin-1 or GSK'872. (B) Cell viability in presence of dox 1000 ng/ml with the different compounds alone or in combination. Z: Z-vad (20 μM); N: necrostatin-1 (30 μM), N+: necrostatin-1 (90 μM), G: GSK'872 (2 μM); G+: GSK'872 (4 μM).

FIG. 5: In vivo DUX4 mediated toxicity leading to weight loss is triggered by Ripk3. (A) Variation of the total body weight gain (in percentage) from the beginning of treatment to animal death. (B) Total running time was measured on a 15° angled treadmill system. (C and D) Weights of the tibialis anterior (TA) and quadriceps (QUA) in the different models. N=4-8 animals/group. Male (C) or female (D). Six-week-old animals were given a 2-day treatment of 10 mg/kg tamoxifen delivered intraperitoneally and the mice were killed 5 days after the last injection CD: DUX4−/+ animals; CDR: DUX4−/+Ripk3−/−; M: Male; F: Female. Data are presented as means±SD; ****: P<0.0001; **: p<0.01; *: p<0.05 by one-way ANOVA with Dunnett's post hoc test. (E) The quadriceps muscles (Female only) were sectioned and labelled with laminin, and the min Feret was calculated for each muscle fibre. (F and G) The min Feret average (F) and the ratio min Feret/Feret was calculated in the quadriceps of CD− and CDR−Cre+ females. (H and I): Hematoxylin and eosin staining of CD−Cre+ (H) and CDR−Cre+ (I) muscle cross sections.

FIG. 6: Expression of several DUX4 downstream genes is lower in Ripk3−/− animals (A, B, C, D, E, F). Expression of necroptotic genes (Ripk1, Ripk3 and Mlk1) and DUX4-network genes (Tm7sf4, mDuxb1 and Wfdc3) were measured in the quadriceps. Data represents the mean±SD, n=4-8 animals/group. Six-week-old female were given a 2-day treatment of 10 mg/kg tamoxifen delivered intraperitoneally and the mice were killed 5 days after the last injection. CD: DUX4−/+ animals; CDR: DUX4−/+Ripk3−/−.

FIG. 7: RIPK3 deficiency ameliorates muscle phenotype in mouse. Representative images of transversed sections of mouse quadriceps labelled with Laminin and CD68 (A) or Laminin and IgG uptake (B). Quantification of CD68− (C) or IgG-uptake− (D) positive areas. Scale bar 200 μm. Data represents the mean±SEM on 3-5 animals/group. CD: DUX4−/+ animals; CDR: DUX4−/+Ripk3−/−.

FIG. 8: DUX4 expression triggers expression of several genes downstream of DUX4 (A, B, C). Expression of Wfdc3 (A), Dux-Bl (B), and Snx30 (C) was measured by RT-PCR 1 day after addition different concentration of Doxycycline (ng/ml) in iC2C12-DUX4 myoblasts.

FIG. 9: Apoptosis is not involved in iC2C12-DUX4 death. (A) Cell viability on iC2C12-DUX4 myoblasts in the presence of different concentrations of DMSO induced a non-specific increase of cell survival. (B) Caspase 3/7 activity increased after DUX4 expression and is correctly inhibited in the presence of Z-VAD. iC2C12-DUX4 cells were incubated in the presence of Z-Vad (30 μM or 50 μM) or not, with different concentration of doxycycline. Caspase 3/7 activity was measured using the Caspase-Glo 3/7 Assay (Promega) according to the manufacturer's instructions.

FIG. 10: Role of necroptosis in the DUX4 mediated cell death. Several death inhibitors were added at different concentrations alone to iC2C12 myoblasts in presence or not of Doxycycline 200 ng/ml. N: necrostatin1; G: GSK'872; C: cyclosporin A. Cell viability was measured using the adenosine triphosphate (ATP) assay at 24 hours after dox induction.

FIG. 11: Several DUX4 downstream genes are increased in a dose dependent manner after addition of Dox in iC2C12-DUX4 myotubes. Different concentrations of doxycycline (ng/ml) were added in iC2C12-DUX4 myotubes after 4 days of differentiation. The expression of Wfdc3, Dux-Bl and Snx30 was measured 24 hours after addition different concentration of doxycycline.

FIG. 12: Creation of a new transgenic CDR (DUX4−/+Rip−/−) cre-negative or -positive mouse model.

FIG. 13: Ripk3 inhibition decreased DUX4-mediated cell death in vivo Cre-inducible FLExDUX4 mice were injected at the age of 6 weeks with tamoxifen (10 mg/kg for 2 days, IP injection) with or without GSK'872 (2 mg/kg for 5 days, IP injection). Mice were killed at the age of 7 weeks. (A) Total body weight was measured at day 0 and 7. Y-axis represents the weight after tamoxifen at D7 relative to the weight before tamoxifen injection at DO. (BC) Weights of the tibialis anterior (TA) (B) and quadriceps (C) were measured at D7. (DE) Expression levels of 2 genes (Tm7sf4 (D) and DuxBl (E)) downstream of DUX4 were measured in the quadriceps. Y-axis represents arbitrary units relative to Psma2.
* P<0.05, **P<0.01, ***P<0.001, one-way ANOVA followed by a Tukey's multiple comparison test.

FIGS. 14 to 16: An exemplary expression construct encoding shRNAs operably linked to pol III promoters (SEQ ID NO: 60).

FIG. 17: In vivo treatment with galavit (GVT). Galavit (4.5 mg/kg) was injected intraperitoneally daily for 3 consecutive days to 7-8 week old Actal Cre-FLEx DUX4 males. Tamoxifen was intraperitoneally injected at day 0 and 1 (10 mg/kg). Mice were scarified 7 days after the 1st injection. (A)-(C) Variation in weight of tibialis anterior (TA), quadriceps (QUA) and total body weight in treated and untreated animals. (D)-(E) Ratio of total body weight divided by TA or QUA weight in treated and untreated animals. (F)-(H) mRNA expression of several DUX4 downstream genes measured in arbitrary units in treated and untreated animals. Expression of necroptotic genes (Ripk1, Ripk3 and Mlk1) and DUX4-network genes (Tm7sf4, mDuxb1 and Wfdc3) were measured.

FIG. 18: In vivo treatment with dabrafenib (Dab/Dabra). Dabrafenib (30 mg/kg) was given by oral (p.o.) gavage to 4-5 week old Actal Cre-FLEx DUX4 animals 5 times/week for 4 weeks. Tamoxifen was intraperitoneally weakly injected (2 mg/kg). (A)-(F) mRNA expression of several DUX4 downstream genes measured in arbitrary units in treated and untreated animals. Expression of necroptotic genes (Ripk1, Ripk3 and Mlk1) and DUX4-network genes (Tm7sf4, mDuxb1 and Wfdc3) were measured. Males (A-C); Females (D-F). (G) Variation in weight of tibialis anterior (TA) and quadriceps (qua) in treated and untreated animals, expressed as a ratio to total body weight (body). Males (M) and Females (F).

### DETAILED DESCRIPTION

### Definitions

It is to be understood that the terminology used herein describes particular embodiments of the invention only, and is not intended to be limiting.

The articles “a” and “an” are used herein to refer to one or more than one (i.e., to at least one) of the grammatical object of the article. By way of example, “an element” means one element or more than one element.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form or can exist in a non-native environment such as, a host cell.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA or a cDNA may also include introns to the extent that the nucleotide sequence encoding the protein may in some version contain an intron(s).

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

The term “polynucleotide” as used herein is defined as a chain of nucleotides. Furthermore, nucleic acids are polymers of nucleotides. Thus, nucleic acids and polynucleotides as used herein are interchangeable. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR and the like, and by synthetic means.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate the transfer of nucleic acid into cells, such as, for example, polylysine compounds, liposomes, and the like. Examples of viral vectors include, but are not limited to, adenoviral vectors, adeno-associated virus vectors, retroviral vectors, and the like.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., lentiviruses, retroviruses, adenoviruses, and adeno-associated viruses) that incorporate the recombinant polynucleotide.

The term “promoter” as used herein is defined as a DNA sequence recognized by the synthetic machinery of the cell or introduced synthetic machinery, required to initiate the specific transcription of a polynucleotide sequence.

As used herein, the term “promoter/regulatory sequence” means a nucleic acid sequence, which is required for expression of a gene product operably linked to the promoter/regulatory sequence. In some instances, this sequence may be the core promoter sequence and in other instances, this sequence may also include an enhancer sequence and other regulatory elements, which are required for expression of the gene product. The promoter/regulatory sequence may, for example, be one, which expresses the gene product in a tissue-specific manner.

A “constitutive” promoter is a nucleotide sequence which, when operably linked with a polynucleotide which encodes or specifies a gene product, causes the gene product to be produced in a cell under most or all physiological conditions of the cell.

An “inducible” promoter is a nucleotide sequence which, when operably linked with a polynucleotide which encodes or specifies a gene product, causes the gene product to be produced in a cell substantially only when an inducer which corresponds to the promoter is present in the cell.

A “tissue-specific” promoter is a nucleotide sequence which, when operably linked with a polynucleotide encodes or specified by a gene, causes the gene product to be produced in a cell preferentially if the cell is a cell of the tissue type corresponding to the promoter.

In the present application, “antisense oligonucleotide”, or “AON” denotes a single-stranded nucleic acid sequence, either DNA or RNA, which is complementary to a part of a (pre-) mRNA coding a protein which is abnormally expressed in a cell, such as an mRNA encoding RIPK3 or RIPK1 in an FSHD patient.

The terms “abnormal” or “aberrant” when used in the context of organisms, tissues, cells or components thereof, refers to those organisms, tissues, cells or components thereof that differ in at least one observable or detectable characteristic from those organisms, tissues, cells or components thereof that display the “normal” (expected) respective characteristic. Characteristics, which are normal or expected for one cell or tissue type, might be abnormal for a different cell or tissue type. In the context of the present invention, activation of the necroptosis pathway in muscle tissue is abnormal or aberrant.

The term “facioscapulohumeral dystrophy” may also be referred to as facioscapulohumeral type progressive muscular dystrophy, facioscapuloperoneal muscular dystrophy or FSH muscular dystrophy. Two types of facioscapulohumeral muscular dystrophy are known: type 1 (FSHD1) and type 2 (FSHD2). The two types have the same signs and symptoms and are distinguished by their genetic cause. The present invention may be used to treat FSHD1 and/or FSHD2. FSHD may also be categorised as adult-onset (including FSHD that begins in adolescence) and infantile-onset forms. The present invention may be used to treat adult-onset FSHD and/or infantile-onset FSHD.

The term “necroptosis” may be defined as necrotic cell death that is dependent on receptor-interacting protein kinase-3 (RIPK3) and/or receptor-interacting protein kinases 1 (RIPK1). Necroptosis is regulated by multiple steps of post-transcriptional modifications including phosphorylation and ubiquitination (McQuade, 2013), leading in particular to the pseudokinase mixed-lineage kinase domain-like (MLKL) phosphorylation and its translocation to the membrane and the disruption of the plasma membrane (for a review see Silke, 2015). Morphological features of necroptosis include increased cell volume, organelle shrinkage, plasma membrane disintegration. By comparison, apoptosis is characterised cell shrinkage, chromatin condensation, the formation of apoptotic bodies and phagocytosis by adjacent cells.

The terms “patient,” “subject,” “individual,” and the like are used interchangeably herein, and refer to any animal or cells thereof whether in vitro or in situ, amenable to the methods described herein. The patient is preferably a mammal. The mammal may be a commercially farmed animal, such as a horse, a cow, a sheep or a pig, a laboratory animal, such as a mouse or a rat, or a pet, such as a cat, a dog, a rabbit or a guinea pig. The patient is more preferably human.

The terms “treat,” “treated,” “treating,” or “treatment” as used herein refer to both therapeutic treatment and prophylactic or preventative measures, wherein the object is to prevent or slow down (lessen) an undesired physiological condition, disorder or disease, or to obtain beneficial or desired clinical results. Beneficial or desired clinical results include, but are not limited to, alleviation of symptoms; diminishment of the extent of the condition, disorder or disease; stabilization (i.e., not worsening) of the state of the condition, disorder or disease; delay in onset or slowing of the progression of the condition, disorder or disease; amelioration of the condition, disorder or disease state; and remission (whether partial or total), whether detectable or undetectable or enhancement or improvement of the condition, disorder or disease. Treatment includes eliciting a clinically significant response without excessive levels of side effects.

A “therapeutic” treatment is a treatment administered to a subject who exhibits signs of pathology, to diminish or eliminate those signs.

An “effective amount” of a compound is that amount of compound which is sufficient to provide a beneficial effect to the subject to which the compound is administered. The phrase “therapeutically effective amount”, as used herein, refers to an amount that is sufficient or effective to prevent or treat (delay or prevent the onset of, prevent the progression of, inhibit, decrease or reverse) a disease or condition, including alleviating symptoms of such diseases. An “effective amount” of a delivery vehicle is that amount sufficient to effectively bind or deliver a compound.

All publications, patents and patent applications cited herein are incorporated by reference in their entirety.

### Gene Silencing

The present invention relates to a mechanism of gene silencing useful for treating a subject suffering from a disease resulting from the abnormal (or aberrant) expression of a protein. Specifically, the invention relates to a mechanism of gene silencing useful for treating a disease resulting from the aberrant expression of DUX4 and components of the necroptosis pathway in muscle tissue, such as FSHD. The skilled person will appreciate that the necroptosis pathway may be silenced by numerous means, including the use of antisense oligonucleotides (AONs), for example, AONs delivered by gene therapy, small molecule inhibitors, gene-editing tools, for example, to replace RIPK3 and/or RIPK1 with modified genes encoding kinase-dead proteins, and inhibitory antibodies. The present invention therefore also includes any possible means for inhibiting the necroptosis pathway for the treatment of FSHD.

### Antisense Oligonucleotides

In one embodiment, the present invention relates to an antisense oligonucleotide (AON) useful for treating a subject suffering from a disease resulting from the abnormal (or aberrant) expression of a protein. Specifically, the invention relates to an AON useful for treating a disease resulting from the aberrant expression of DUX4 and components of the necroptosis pathway in muscle tissue. Also provided is an AON and a nucleic acid molecule encoding said AON. In a specific embodiment, the subject has FSHD and the mRNA (or pre-mRNA) targeted by the AON encodes a component or regulator of the necroptosis pathway. Preferably, the mRNA is a RIPK3 mRNA, a RIPK1 mRNA or a MLKL mRNA. Preferably, the mRNA is selected from SEQ ID NO: 1 to 17. Genes to be silenced by the inhibitory sequences of the invention can be considered as target genes. Accordingly, exemplary target genes include gene encoding components of the necroptosis pathway, such as RIPK3, RIPK1 or MLKL.

An AON may be used to silence target gene expression and/or activity in a target tissue or cell. In a particular embodiment, the target tissue is a muscle tissue, such as skeletal muscle. In a particular embodiment, the target cell is a myoblast, a myotube, or a mature myofibre.

A variety of mechanisms to silence gene expression or activity are encompassed by the present invention. The term silencing used herein encompasses the reduction, inhibition or downregulation of gene expression, the reduction, inhibition or downregulation of transcription, the reduction, inhibition or downregulation of translation, and/or the reduction inhibition or downregulation of protein activity. Specifically, the term silencing used herein encompasses the reduction, inhibition or downregulation of gene expression, transcription, translation and/or protein activity of or more components involved in the necroptosis pathway. Of particular interest are RIPK3, RIPK1 and MLKL coding sequences (genes, pre-mRNA and mRNA) and proteins. The reduction, inhibition or downregulation can be direct or indirect.

The reduction, inhibition or downregulation can be complete or partial. Silencing as described herein can thus be considered to encompass a 10% reduction, a 20% reduction, a 30% reduction, a 40% reduction, a 50% reduction, a 60% reduction, a 70% reduction, a 80% reduction, a 90% reduction, or a 100% reduction in gene expression, transcription, translation and/or protein activity compared to a suitable control. In one embodiment, a control may be the test subject prior to treatment.

The AON of the invention comprises a sequence that acts to prevent or alter the production of proteins. Specifically, AONs may silence gene expression, transcription, translation and/or protein activity of a target. In some embodiments of the invention, the AON sequence comprises a single or double-stranded RNA, ncRNA, shRNA, siRNA or a miRNA.

Broadly, AONs may induce the degradation of a target mRNA and/or physically prevent or inhibit the progression of splicing or the translational machinery. Inhibiting the progression of the splicing machinery causes exon skipping or intron retention which induces a frameshift into a coding sequence. Thus, the AON of the invention may cause exon skipping or intron retention in any component of the necroptosis pathway, including Ripk1, Ripk3 or Mlk1.

Representative AON types include, but are not limited to, oligodeoxyribo-nucleotides, oligoribonucleotides, morpholinos, tricyclo-DNA-antisense oligonucleotides, tricyclophosphorothioate DNA oligonucleotides, locked nucleic acids (LNA) or any conjugates thereof. Exemplary conjugates include peptide-conjugated or nanoparticle-complexed AONs.

Preferably, the AON is a small hairpin RNA (shRNA). shRNAs are sequences of RNA that include a region of internal hybridization that creates a hairpin structure. shRNA molecules are processed within the cell to form siRNA which in turn knocks down gene expression. The benefit of shRNA is that they can be incorporated into plasmid vectors and integrated into genomic DNA for longer-term or stable expression, and thus longer knockdown of the target mRNA.

In specific embodiments of the invention, the AON targets a pre-mRNA or mRNA encoding a component or modulator of the necroptosis pathway, optionally causing the functional inactivation and/or degradation of the mRNA. For example, the AON may target a RIPK3 mRNA, RIPK1 mRNA or MLKL mRNA. In a preferred embodiment, the mRNA is a human mRNA. For example, the AON may target an mRNA having the sequence of any one of SEQ ID NO: 1 to 17 or a variant, fragment or derivative thereof. In a preferred embodiment, the mRNA is a human mRNA. For example, the AON may target an mRNA having the sequence of any one of SEQ ID NO: 1 to 10 or a variant, fragment or derivative thereof. For example, the AON may target an mRNA having at least 70% sequence identity, at least 75% sequence identity, at least 80% sequence identity, at least 85% sequence identity, at least 90% identity, at least 95% sequence identity, at least 98% sequence identity, or at least 99% sequence identity to an mRNA molecule having the sequence of SEQ ID NO: 1 to 10. In this context, target means an mRNA molecule comprising a sequence which is complementary to the AON.

In a preferred embodiment, the AON targets a contiguous sequence within SEQ ID NO 1 to 17. Preferably the sequence is a sequence within SEQ ID NO: 18 to 35. For example, the target sequence may be at least 10, at least 11, at least 12, at least 13, at least 14, at least 15, at least 16, at least 17, at least 18, or least 19 nucleotides of SEQ ID NO: 18 to 35. In this context, the term “target” refers to an endogenous RNA sequence that is 100% complementary to a contiguous sequence of the AON.

In a preferred embodiment, the AON targets an RNA molecule comprising or consisting of sequence listed in Table 1, or a fragment or variant thereof. For example, the AON comprises a sequence that is 100% complementary to at least 10, at least 11, at least 12, at least 13, at least 14, at least 15, at least 16, at least 17 or at least 18 contiguous nucleotides of any one of SEQ ID NO: 18 to 35. Preferably, the AON comprises a sequence that is 100% complementary to any sequence selected from SEQ ID NO: 18 to 35.

TABLE 1

| Targeted | | |
|---|---|---|
| element | AON target | SEQ ID |
| hRIPK3 mRNA | 5′ GAUGUCGUGCGUCAAGUUA 3′ | SEQ ID NO: 18 |
| hRIPK3 mRNA | 5′ GGCAAGUCUGGAUAACGAA 3′ | SEQ ID NO: 19 |
| hRIPK3 mRNA | 5′ GCUGCUGUCUCCACGGUAA 3′ | SEQ ID NO: 20 |
| hRIPK1 mRNA | 5′ GAAUGUCAUUAAGAUGAAA 3′ | SEQ ID NO: 21 |
| hRIPK1 mRNA | 5′ GGGCGAUAUUUGCAAAUAA 3′ | SEQ ID NO: 22 |
| hRIPK1 mRNA | 5′ CGUGAAGAGUUUAAAGAAA 3′ | SEQ ID NO: 23 |
| hMLKL mRNA | 5′ GGUGUGAAGAGAUGAAAUA 3′ | SEQ ID NO: 24 |
| hMLKL mRNA | 5′ GGAGAUCCCGCAAGAGCAA 3′ | SEQ ID NO: 25 |
| hMLKL mRNA | 5′ GGAUGAAAUCUUAAAGAAA 3′ | SEQ ID NO: 26 |
| mRipk3 mRNA | 5′ GGUAGACAAGACUUCACUA 3′ | SEQ ID NO: 27 |
| mRipk3 mRNA | 5′ GAUGUCUUCUGUCAAGUUA 3′ | SEQ ID NO: 28 |
| mRipk3 mRNA | 5′ GUGAACUCGAAGAAGAUAU 3′ | SEQ ID NO: 29 |
| mRipk1 mRNA | 5′ GGAAGGUGUCCUUGUGUUA 3′ | SEQ ID NO: 30 |
| mRipk1 mRNA | 5′ GUGAGCAGCACCACUAAGA 3′ | SEQ ID NO: 31 |
| mRipk1 mRNA | 5′ GAAAGAGUAUCCAGAUCAA 3′ | SEQ ID NO: 32 |
| mMlkl mRNA | 5′ GGGACAGAUCAUCAAGUUA 3′ | SEQ ID NO: 33 |
| mMlkl mRNA | 5′ GAGGAAGACGGAAAUGAAA 3′ | SEQ ID NO: 34 |
| mMlkl mRNA | 5′ GCAGAGAGAUCCAGUUCAA 3′ | SEQ ID NO: 35 |

When the AON is an shRNA, the total length of the stem-loop structure is generally from 20 to 75 nucleotides in length, for example, from 25 to 65 nucleotides in length, from 35 to 55 nucleotides in length or from 40 to 50 nucleotides in length. The total length of the shRNA may be about 35, about 40, about 45, or about 50 nucleotides in length depending on the targeted sequences within the target mRNA. The stem is generally from 10 to 35, from 15 to 30 or from 20 to 25 nucleotides in length. The stem may comprise a perfectly complementary duplex (but for any 3′ tail), however, bulges or interior loops may be present, and even preferred, on either arm of the stem. The number of such bulges and asymmetric interior loops are preferably few in number (e.g., 1, 2 or 3) and are about 3 nucleotides or less in size. The terminal loop portion may comprise about 4 or more nucleotides, but preferably not more than about 25. More particularly, the loop portion will preferably be 6-15 nucleotides in size.

As described herein, the stem region of the shRNAs comprises a passenger strand and guide strand, whereby the guide strand contains a sequence complementary to the target mRNA transcript encoded by the target gene. The guide strand is 100% complementary to the mRNA. However, mismatches between passenger and guide strands are permitted. Preferably, the G-C content and matching of guide strand and passenger strand are carefully designed for thermodynamically-favourable strand unwind activity with or without endonuclease cleavage. Furthermore, the specificity of the guide strand is preferably confirmed via a BLAST search (www.ncbi.nim.nih.gov/BLAST).

Exemplary shRNAs that target RIPK3 mRNA may be encoded by the sequences shown in FIG. 14. In particular, the sequence of an shRNA targeting exon 6 of RIPK3 is shown at nt 364-310 of FIG. 14, the sequence of an shRNA targeting exon 1 of RIPK3 is shown at nt 625-671 of FIG. 14, and the sequence of an shRNA targeting exon 2-3 of RIPK3 is shown at nt 967-1013 of FIG. 14.

The invention provides that the expression level of multiple target genes may be modulated using the methods and shRNAs described herein. For example, the invention provides a first set of shRNAs designed to include a sequence (a guide strand) that reduces the expression level of a first target gene, whereas a second set of shRNAs may be designed to include a sequence (a guide strand) that is designed to reduce the expression level of a second target gene. The different sets of shRNAs may be expressed and reside within the same, or separate, preliminary transcripts. In certain embodiments, such multiplex approach, i.e., the use of the shRNAs described herein to modulate the expression level of two or more target genes, may have an enhanced therapeutic effect on a patient. For example, if a patient is provided with the shRNAs described herein to treat, prevent, or ameliorate the effects of FSHD, it may be desirable to provide the patient with two or more types of shRNAs, which are designed to reduce the expression level of multiple genes that are implicated in FSHD.

AONs of the invention may be delivered in vivo alone or in association with a vector. The precursor sequences (or constructs) encoding the AON may be introduced into host cells using any of a variety of techniques and delivery vehicles well-known in the art. For example, infection with a viral vector comprising one or more constructs may be carried out, wherein such viral vectors preferably include replication-defective retroviral vectors, adenoviral vectors, adeno-associated vectors, lentiviral vectors, or measles vectors. In addition, transfection with a plasmid comprising one or more constructs may be employed. Such plasmids may be present as naked DNA or in association with, for example, a liposome (e.g., an immunoliposome). Further, the delivery vehicle may consist of immunolipoplexes, targeted nanoparticles, targeted liposomes, cyclodextrins, nanoparticles, aptamers, dendrimers, chitosan, or pegylated derivatives thereof. The nature of the delivery vehicle may vary depending on the target host cell.

In vivo delivery of the AON-encoding constructs may be carried out using any one of a variety of techniques, depending on the target tissue. Delivery may be, for example, achieved by direct injection, inhalation, intravenous injection or other physical methods (including via micro-projectiles to target visible and accessible regions of tissue (e.g., with naked DNA). Administration may further be achieved via syringe needles, trocars, cannulas, catheters, etc., as appropriate.

Further, the present invention encompasses methods of using the nucleic acid sequences and AONs described herein to prevent, treat and/or ameliorate the effects of one or more medical conditions, including FSHD. For example, the invention provides that the shRNAs described herein may be used to reduce the expression level of one or more target genes that are implicated in FSHD. For example, the shRNAs may be used to reduce the expression level of certain target genes that regulate necroptosis such as RIPK3, RIPK1 and MLKL.

In addition to therapeutic applications, the AONs described herein may be used in research-oriented applications. Cultured cells suitable as hosts for the precursor sequences encoding the AONs of the present invention include both primary cells and cell lines. These cells may be human cells, including human stem cells or non-human animal cells. A construct of the invention encoding the AONs may be introduced into cultured cells to inactivate a specific gene of unknown function. Silencing the gene of interest using the AONs can be used as an approach to assess its function. Of course, the AONs of the invention may be introduced into non-human animal cells to produce a model experimental animal. In the case of experimental animals, the AONs can be used for large scale analysis of gene function.

Commercial providers such as Ambion Inc. (Austin, Tex.), Darmacon Inc. (Lafayette, Colo.), InvivoGen (San Diego, Calif.), and Molecular Research Laboratories, LLC (Herndon, Va.) generate custom inhibitory RNA molecules. In addition, commercially kits are available to produce custom siRNA molecules, such as SILENCER™ siRNA Construction Kit (Ambion Inc., Austin, Tex.) or psiRNA System (InvivoGen, San Diego, Calif.).

### Expression Constructs

A nucleic acid molecule of the invention may be provided in the form of an expression construct. An expression construct includes a control sequence(s) operably linked to the nucleic acid molecule as described above. Thus, the expression construct allows expression of the gene inhibitory sequence of the invention in vivo. The term “operably linked” refers to a juxtaposition wherein the components described are in a relationship permitting them to function in their intended manner. A control sequence “operably linked” to a gene inhibitory sequence is ligated in such a way that expression of the gene inhibitory sequence is achieved under conditions compatible with the control sequences. These expression constructs, in turn, are typically provided within vectors (e.g., plasmids or recombinant viral vectors). Such an expression cassette may be administered directly to a host subject.

An expression cassette of the invention can be assembled into a vector of the invention and delivered to the muscles of a test animal, such as a mouse, and the effects observed and compared to a control.

### Promoters

The nucleic acid molecule may be operatively linked to a regulatory sequence allowing or improving the expression of the encoded sequence. Regulatory sequences include promoters, enhancer internal ribosome entry sites (IRES), sequences encoding protein transduction domains (PTD) and the like.

When the regulatory sequence is a promoter, the promoter may be cellular, viral, fungal, plant or synthetic. Most preferred promoters for use in the present invention shall be functional in muscle cells. The AON may be transcribed with an RNA Polymerase III (Pol III) using a promoter which specifically binds to Pol III. Suitable Pol III promoters may be selected from U1, U2, U6, 7SK, or H1, or any variant thereof. For example, several variants of U6 have been characterised, including U6-1 to U6-9 disclosed in Domitrovich, A. et al. Of these promoters, U6-1 and U6-9 were used in Malerba, et al. Accordingly, the AON of the invention may be transcribed using a U6 promoter selected from U6-1, U6-2, U6-3, U6-4, U6-5, U6-6, U6-7, U6-8 or U6-9. Preferably, the U6 promoter is U6-1. In some embodiments, the promoters are those shown in FIG. 14. Alternatively, the AON may be transcribed using an RNA Polymerase II (Pol II) promoter, such as U7

The promoter may be constitutive or conditionally active. Examples of regulated promoters include, without limitation, Tet on/off element-containing promoters, rapamycin-inducible promoters and metallothionein promoters.

Examples of ubiquitous promoters include viral promoters, particularly the CMV promoter, the RSV promoter, the SV40 promoter, hybrid CBA (Chicken-beta actin/CMV) promoter, etc. and cellular promoters such as the PGK (phosphoglycerate kinase) or EF1 alpha (Elongation Factor 1 alpha) promoters.

The promoter may alternatively be a muscle-preferred or muscle-specific promoter or variant thereof, such as a skeletal muscle-preferred or a skeletal muscle-specific promoter. Muscle-preferred expression can be defined as expression that is present in muscle to a greater extent than in other cell types. Muscle-specific expression may be defined as expression that is only present in muscle, and not in other cell types. Muscle-specific expression may be defined as expression that is more than about 10 times greater, 20 times greater, 50 times greater or 100 or more times greater in skeletal muscle than in other cell types. Expression in muscle and other cell types can be measured by any suitable standard technique known to the person skilled in the art. For example, RNA expression levels can be measured by quantitative real-time PCR. Protein expression can be measured by western blotting or immunohistochemistry.

Regulatory sequences may be based on promoters derived from muscle-specific genes. For example, muscle-specific control elements include but are not limited to those derived from the actin and myosin gene families, such as from the myoD gene family (See Weintraub, 1990), the myocyte-specific enhancer binding factor MEF-2 (Cserjesi and Olson, 1991) control elements derived from the human skeletal actin gene (Muscat et al., 1987), the cardiac actin gene, muscle creatine kinase sequence elements (See Johnson, 1989) and the murine creatine kinase enhancer (mCK) element, control elements derived from the skeletal fast-twitch troponin C gene, the slow-twitch cardiac troponin C gene and the slow-twitch troponin I gene: hypoxia-inducible nuclear factors (Semenza et al., 1990), steroid-inducible elements and promoters including the glucocorticoid response element (GRE) (See Mader and White, 1993), and other control elements. Alternatively, synthetic, modified, or hybrid promoters such as C5-12 CK6-CK9, tMCK or a modular muscle hybrid (MH) promoter may be used (Lui, 2004; Hauser M. A, 2000; Himeda C. L., 2011; Wang B, 2008). Endogenous tissue-specific promoters may be modified for expression in an AAV vector.

Introduction of such constructs into host cells may be effected under conditions whereby the two or more AON molecules that are contained within a precursor transcript initially reside within a single primary transcript, such that the separate AON molecules (e.g., each comprising its own stem-loop structure) are subsequently excised from such precursor transcript by an endogenous ribonuclease.

The invention shall not be restricted to the use of any single promoter, especially since the invention may comprise two or more AONs (i.e., a combination of effectors), including but not limited to shRNA.

One or more other regulatory elements may also be present as well as the promoter. For example, the promoter of the invention can be used in tandem with one or more further promoters or enhancers or locus control regions (LCRs).

A nucleic acid molecule of the invention may be administered by a gene therapy vector, liposome, nanoparticle (for example, a polymeric nanoparticle, solid lipid nanoparticle, or compacted DNA nanoparticle), a dendrimer, polyplex, or polymeric micelles.

### Vectors

When the nucleic acid molecule of the invention is administered by a gene therapy vector, the vector may be of any type. For example, the vector may be a plasmid vector or a minicircle DNA vector.

Typically, vectors of the invention are viral vectors. The viral vector may be based on the herpes simplex virus, adenovirus or lentivirus. Adeno-associated virus (AAV) vectors or derivatives thereof are particularly attractive as they are generally non-pathogenic; the majority of people have been infected with this virus without adverse effects.

Vectors of the invention typically comprise two inverted terminal repeats (ITRs), preferably one at each end of the genome. An ITR sequence acts in cis to provide a functional origin of replication and allows for the integration and excision of the vector from the genome of a cell. The AAV genome typically comprises packaging genes, such as rep and/or cap genes which encode packaging functions for an AAV viral particle. The rep gene encodes one or more of the proteins Rep78, Rep68, Rep52 and Rep40 or variants thereof. The cap gene encodes one or more capsid proteins such as VP1, VP2, and VP3 or variants thereof. These proteins make up the capsid of an AAV viral particle.

The AAV genome may be from any naturally derived serotype or isolate or clade of AAV. The AAV vector may be capsid-free. When the AAV vector does encode a capsid, the serotype can be selected by one skilled in the art depending on the target cell that must be transduced. In a particular embodiment, the target cell is of the muscle lineage, and the capsid of the AAV vector is from serotype 1, 6, 8, 9 or rhesus serotype 74 (rh74) of AAV. In a further particular embodiment, the AAV vector is a pseudotyped vector, i.e. its genome and capsid are derived from AAVs of different serotypes. For example, the pseudotyped AAV vector may be a vector whose genome is derived from the AAV2 serotype, and whose capsid is derived from the AAV1, 6, 8, 9 or rh74 serotype or variants thereof. In addition, the genome of the AAV vector may either be a single stranded or self-complementary double-stranded genome (McCarty et al., 2001; McCarty et al., 2008).

Preferably the AAV genome will be derivatized for administration to patients. Typically, it is possible to truncate the AAV genome significantly to include minimal viral sequence yet retain the above function. Derivatisation reduces the risk of recombination of the vector with the wild-type virus, and avoids triggering a cellular immune response by the presence of viral gene proteins in the target cell. A derivative may be a chimeric, shuffled or capsid-modified derivative of one or more naturally occurring AAV viruses.

Chimeric, shuffled or capsid-modified derivatives will be typically selected to provide one or more desired functionalities for the viral vector. Thus, these derivatives may display increased efficiency of gene delivery, decreased immunogenicity (humoral or cellular), an altered tropism range and/or improved targeting of a particular cell type compared to an AAV viral vector comprising a naturally occurring AAV genome, such as that of AAV2.

Increased efficiency of gene delivery may be affected by improved receptor or co-receptor binding at the cell surface, improved internalization, improved trafficking within the cell and into the nucleus, improved uncoating of the viral particle and improved conversion of a single-stranded genome to double-stranded form. Increased efficiency may also relate to an altered tropism range or targeting of a specific cell population, such that the vector dose is not diluted by administration to tissues where it is not needed.

Chimeric capsid proteins include those generated by recombination between two or more capsid coding sequences of naturally occurring AAV serotypes. This may be performed for example by a marker rescue approach in which non-infectious capsid sequences of one serotype are cotransfected with capsid sequences of a different serotype, and directed selection is used to select for capsid sequences having desired properties. The capsid sequences of the different serotypes can be altered by homologous recombination within the cell to produce novel chimeric capsid proteins.

Chimeric capsid proteins may be generated by engineering the capsid protein sequences to transfer specific capsid protein domains, surface loops or amino acid residues between different capsid proteins. Shuffled capsid proteins may be generated by DNA shuffling or by error-prone PCR.

The sequences of capsid genes may also be genetically modified to introduce specific deletions, substitutions or insertions to the native wild-type sequence. In particular, capsid genes may be modified by the insertion of a sequence of an unrelated protein or peptide within an open reading frame of a capsid coding sequence, or at the N- and/or C-terminus of a capsid coding sequence. The unrelated protein or peptide may advantageously be one which acts as a ligand for a particular cell type, thereby conferring improved binding to a target cell or improving the specificity of targeting of the vector to a particular cell population. The unrelated protein may also be one that assists purification of the viral particle as part of the production process i.e. an epitope or affinity tag. The site of insertion will typically be selected so as not to interfere with other functions of the viral particle e.g. internalization, trafficking of the viral particle. The skilled person can identify suitable sites for insertion based on their common general knowledge.

The invention additionally encompasses the provision of sequences of an AAV genome in a different order and configuration to that of a native AAV genome. The invention also encompasses the replacement of one or more AAV sequences or genes with sequences from another virus or with chimeric genes composed of sequences from more than one virus. Such chimeric genes may be composed of sequences from two or more related viral proteins of different viral species.

AAV viruses are replication-incompetent. Therefore helper virus functions, preferably adenovirus helper functions, will typically also be provided on one or more additional constructs to allow for AAV replication.

For the avoidance of doubt, the invention also provides an AAV viral particle comprising a vector of the invention. The AAV particles of the invention include transcapsidated forms wherein an AAV genome or derivative having an ITR of one serotype is packaged in the capsid of a different serotype. The AAV particles of the invention also include mosaic forms wherein a mixture of unmodified capsid proteins from two or more different serotypes makes up the viral envelope. The AAV particle also includes chemically modified forms bearing ligands adsorbed to the capsid surface. For example, such ligands may include antibodies for targeting a particular cell surface receptor.

The present invention also encompasses DNA sequences that are incorporated into AAV genomes (or vectors) devoid of capsids (i.e., AAV0). Such vectors are described in detail in WO 2012/123430. Capsid-free AAV vectors can be delivered into target cells, tissues, organs, or subjects for efficient expression of a protein, RNA or DNA of interest without relying on a viral capsid to facilitate the uptake process.

Accordingly, the invention provides for a minimal rAAV0 vector which has two AAV inverted terminal repeats (ITRs) and a nucleotide sequence of interest (for example an expression cassette). Each ITR comprises an interrupted (or noncontiguous) palindromic sequence. Specifically, each ITR comprises a sequence having a first segment and a third segment that are identical when read 5′ to 3′ but hybridize when placed against each other. A second segment separates the identical segments. Such ITRs form hairpin structures. The nucleotide sequence of interest can, in particular, be an expression cassette comprising at least one promoter operatively linked to an exogenous DNA sequence, and flanked on each end by one ITR. The rAAV0 vector does not encode capsid proteins, and the rAAV0 vector is not encapsidated. The rAAV0 vector can be single-stranded, double-stranded, or duplex with one or both ends covalently linked via the ITR palindrome. In a preferred embodiment, the rAAV0 vector is single-stranded.

For example, in one aspect, an rAAV0 vector can deliver an AON, a nucleic acid sequence encoding an AON, or a nucleic acid encoding a gene-editing tool.

One advantage of using an AAV0 vector is that no anti-capsid response will occur. This can be confirmed, if desired, by multiple injections and by measurement of antibody response or of specific T cell activation or proliferation. In addition, it is expected that even immunogenicity against a protein product of the transgene will be reduced if delivered by a single-stranded rAAV0 because single strand AAV vectors are less capable of eliciting a strong innate immune response than double-stranded AAV vectors.

Capsid-less rAAV0 vectors are produced using cells comprising a nucleotide sequence of interest (e.g. an expression cassette comprising usually at least one promoter operatively linked to an exogenous DNA except for the delivery of a pure DNA template) positioned between two ITRs. The cells either already contain Rep or are transduced with a vector to contain Rep and are then grown under conditions permitting replication and release of DNA comprising the ITRs and the expression cassette and constituting the rAAV0 vector. The rAAV0 vector can then be collected and purified from the cells or supernatant as free released rAAV0 vector or as exosomes or microparticles.

The invention additionally provides a host cell comprising a vector or AAV viral vector or particle of the invention.

The vector of the invention may be prepared by standard means known in the art for the provision of vectors for gene therapy. Thus, well established public domain transfection, packaging and purification methods can be used to prepare a suitable vector preparation.

All of the above additional constructs may be provided as plasmids or other episomal elements in the host cell. Alternatively, one or more constructs may be integrated into the genome of the host cell.

### Small Molecule Inhibitors

Also provided are small molecules that inhibit the necroptosis pathway, leading to reduced muscle atrophy. For example, small molecules of the invention include molecules that block the enzymatic activity or function of one or more RIP kinases, such as RIPK3 or RIPK1, or MLKL. Preferably, the target proteins are human RIPK3, human RIPK1 and/or human MLKL.

Suitable small molecule inhibitors of RIPK1 and/or RIPK3 are listed in Table 2.

TABLE 2
Ripk1 and/or Ripk3 inhibitors

| | |
|---|---|
| 6E11 | 2-[4-(benzyloxy)phenyl]-2,5-dihydroxy-7-methoxy-3,4-dihydro- |
| | 2H-1-benzopyran-4-one |
| compound 21 | 1-[3-[5-(3-aminophenyl)-1H-pyrrolo[2,3-b]pyridin-3-yl]phenyl]- |
| | 3-[2-fluoro-5-(trifluoromethyl)phenyl]urea |
| compound 22 | 7 oxo 2,4,5,7 tetrahydro 6H-pyrazolo[3,4 c]pyridine |
| compound 27 | 1-[4-(4-aminofuro[2,3-d]pyrimidin-5-yl)phenyl]-3-[2-fluoro-5- |
| | (trifluoromethyl)phenyl]urea |
| dabrafenib | N-{3-[5-(2-aminopyrimidin-4-yl)-2-tert-butylthiazol-4-yl]-2- |
| | fluorophenyl}-2,6-difluorobenzenesulfonamide |
| DNL747 | |
| fostamatinib | [6-[[5-fluoro-2-(3,4,5-trimethoxyanilino)pyrimidin-4-yl]amino]- |
| | 2,2-dimethyl-3-oxopyrido[3,2-b][1,4]oxazin-4-y]methyl |
| | dihydrogen phosphate |
| galavit | 3-aminophthalhydrazide monosodium salt |
| GSK2982772 | 5-benzyl-N-[(3S)-5-methyl-4-oxo-2,3-dihydro-1,5-benzoxazepin-3-yl]- |
| | 1H-1,2,4-triazole-3-carboxamide |
| GSK3145095 | (S)-5-benzyl-N-(7,9-difluoro-2-oxo-2,3,4,5-tetrahydro-1H- |
| | benzo[b]azepin-3-yl)-1H-1,2,4-triazole-3-carboxamide |
| GSK840 | [4-(5-Methylcarbamoyl-benzoimidazol-1-yl)-phenyl]-acetic acid |
| | tert-butyl ester |
| GSK843 | 3-Benzothiazol-5-yl-7-(2,5-dimethyl-2H-pyrazol-3-yl)-thieno[3,2- |
| | c]pyridin-4-ylamine |
| GSK872 | Benzothiazol-5-yl-[6-(propane-2-sulfonyl)-quinolin-4-yl]-amine |
| GSK963 | 2,2-dimethyl-1-[(3S)-3-phenyl-3,4-dihydropyrazol-2-yl]propan-1-one |
| GW440139B | 4-methyl-3-[(7-pyridin-2-ylquinolin-4-yl)amino]phenol |
| HS-1371 | Quinoline, 4-(4-methylphenoxy)-7-[1-(4-piperidinyl)-1H-pyrazol-4-yl]- |
| necrostatin-1 | 5-(1H-indol-3-ylmethyl)-3-methyl-2-sulfanylideneimidazolidin-4-one |
| necrostatin-1s | 7-Cl—O-Nec-1) (5-[(7-chloro-1H-indol-3-yl)methyl]-3- |
| | methylimidazolidine-2,4-dione |
| necrostatin-3 | 3R,3aR)-rel-2-acetyl-3,3a,4,5-tetrahydro-3-(4-methoxyphenyl)-8- |
| | methoxy-2H-benz[g]indazole |
| necrostatin-3 | 1-([3S,3aS]-3-[3-fluoro-4-[trifluoromethoxy]phenyl]-8-methoxy- |
| analog | 3,3a,4,5-tetrahydro-2H-benzo[g]indazol-2-yl)-2-hydroxyethanone |
| necrostatin-4 | (S)-N-(1-[2-chloro-6-fluorophenyl]ethyl)-5-cyano-1-methyl-1H- |
| | pyrrole-2-carboxamide |
| necrostatin-5 | 5-(1H-indol-3-ylmethyl)-2-thioxo-4-imidazolidinone |
| pazopanib | 5-[[4-[(2,3-Dimethyl-2H-indazol-6-yl)méthylamino]-2- |
| | pyrimidinyl]amino]-2-methylbenzolsulfonamide |
| PN10 | 5-[(7-chloro-1H-indol-3-yl)methyl]-3-[4-[3-(2-imidazo[1,2- |
| | b]pyridazin-3-ylethynyl)-4-methylphenyl]butyl]imidazolidine- |
| | 2,4-dione |
| ponatinib | 3-(2-imidazo[1,2-b]pyridazin-3-ylethynyl)-4-methyl-N-[4-[(4- |
| | methylpiperazin-1-yl)methyl]-3-(trifluoromethyl)phenyl]benzamide |
| RIPA-56 | N-benzyl-N-hydroxy-2,2-dimethylbutanamide |
| RIPK1 inhibitor 22b | 1-(5-{4-Amino-7-ethyl-7H-pyrrolo[2,3-d]pyrimidin-5-yl}-2,3- |
| | dihydro-1H-indol-1-yl)-2-[3-(trifluoromethoxy)phenyl]ethan-1-one |
| RIPK3 inhibitor 18 | N-{4-[(2-cyclopropaneamidopyridin-4-yl)oxy]-2,3-dimethylphenyl}- |
| | 1-(4-fluorophenyl)-2-oxo-1,2-dihydropyridine-3-carboxamide |
| RIPK3 inhibitor 42 | N-(6-(3-(3-(3-Bromophenyl)ureido)-4-fluorophenoxy)benzo[d]thiazol- |
| | 2-yl)cyclopropanecarboxamide |
| SZM594 | N-[6-(4-fluoro-3-{2-[3-(trifluoromethyl)phenyl]acetamido}phenoxy)- |
| | 1,3-benzothiazol-2-yl]cyclopropanecarboxamide |
| TAK-632 | N-[7-cyano-6-[4-fluoro-3-[[2-[3- |
| | (trifluoromethyl)phenyl]acetyl]amino]phenoxy]-1,3- |
| | benzothiazol-2-yl]cyclopropanecarboxamide |
| Tozasertib, VX-680 | N-[4-[4-(4-Methylpiperazin-1-yl)-6-[(5-methyl-1H-pyrazol-3- |
| or MK-0457 | yl)amino]pyrimidin-2-yl]sulfanylphenyl]cyclopropanecarboxamide |

Small molecule inhibitors of MLKL include are listed in Table 3.

TABLE 3
Mlkl inhibitors

| | |
|---|---|
| GW440139B | 4-methyl-3-[(7-pyridin-2-ylquinolin-4-yl)amino]phenol |
| GW806742X | 3-(4-(Methyl(4-(3-(4-(trifluoromethoxy)phenyl)ureido)phenyl) |
| | amino pyrimidin-2-ylamino)benzenesulfonamide |
| necrosulfonamide | (2E)-N-{4-[(3-methoxypyrazin-2-yl)sulfamoyl]phenyl}- |
| (NSA) | 3-(5-nitrothiophen-2-yl)prop-2-enamide |
| compound1 | 1-[4-[methyl-[2-(3-sulfamoylanilino)pyrimidin-4- |
| | yl]amino]phenyl]-3-[4-(trifluoromethoxy)phenyl]urea |

Preferably, small molecule inhibitors of the invention are selected from dabrafenib, galavit, necrostatin-1s, ponatinib and GSK872.

Small molecules may be delivered to target cells using liposomes, CPPs, injection or combination thereof.

### CRISPR and Guide RNAs

In one embodiment, the silencing mechanism encompasses a mechanism of gene silencing by CRISPR (Clustered Regularly Interspaced Short Palindromic Repeats). In one embodiment, the mechanism of gene silencing by CRISPR involves the use of a guide RNA. The guide RNA may comprise a guide RNA sequence and a tracr RNA. The guide RNA sequence is capable of hybridizing to a target sequence in the DNA to be silenced. The tracr RNA is coupled to the guide RNA sequence. The guide RNA hybridises to the site of the allele and targets a CRISPR-Cas enzyme to said site.

In some embodiments, the guide RNA is between 10-30, or between 15-25, or between 15-20 nucleotides in length. In some embodiments, one guide RNA is used. In some embodiments, two guide RNAs are used. In some embodiments, more than two guide RNAs are used.

Preferably the CRISPR-Cas enzyme is a Type II CRISPR enzyme, for example, Cas-9 (CRISPR associated protein 9). In some preferred embodiments, the Cas-9 enzyme is SaCas-9.

The enzyme complexes with the guide RNA. In one embodiment, the complex targeted to the DNA sequence will bind by hybridization. In one embodiment, the enzyme is active and acts as an endonuclease to cleave the DNA either via activation of the non-homologous end-joining or homologous DNA repair pathway, resulting in a blunt end cut or a nick. In one embodiment, the use of guide RNA or RNAs and the CRISPR enzyme results in the deletion of essential elements of the gene to be silenced, resulting in a non-functional gene. In some embodiments, the gene is not transcribed. In some embodiments, the gene is not translated.

In a preferred embodiment of the invention, the gene to be silenced is RIPK3, RIPK1 or MLKL. Preferably, the gene is a human gene.

In another embodiment, the enzyme is targeted to the DNA of the gene to be silenced but the enzyme comprises one or more mutations that reduce or eliminate its endonuclease activity such that it does not edit the allele but does prevent or reduce its transcription. An example of such an enzyme for use in the invention is dCas-9, which is catalytically dead. In one embodiment, the dCas-9 is dSa-Cas9.

In one embodiment dCas-9 is associated with a transcriptional repressor peptide that can knock down gene expression by interfering with transcription. In a preferred embodiment, the transcriptional repressor protein is Kruppel-associated box (KRAB).

In a related embodiment, the enzyme can be engineered such that it is fused to a transcriptional repressor to reduce or disable its endonuclease function. The enzyme will be able to bind the guide RNA and be targeted to the DNA sequence, but no cleavage of the DNA takes place. The mutant allele may be suppressed, for example, by the shutting down of the promoter or blockage of RNA polymerase.

In a preferred embodiment, the enzyme is dSaCas9-KRAB.

In another embodiment, the transcription repressor may be bound to the tracr sequence. Functional domains can be attached to the tracr sequence by incorporating protein-binding RNA aptamer sequences, as described in Konermann et al. The transcription repressor-tracr sequence complex may be used to target other moieties to a precise gene location as desired.

In another embodiment, the CRISPR mechanism of silencing involves CRISPR base editors to knock out genes by changing single nucleotides to create stop codons (CRISPR-STOP method (Kuscu et al. 2017)).

In another embodiment, the CRISPR mechanism of silencing involves CRISPR activation mediated upregulation of a gene, said upregulation resulting in the silencing of a target gene as described herein. Thus, in an embodiment of the invention, the payload sequence can comprise a dSaCas9-VPR sequence

### ZFPs

In another embodiment of the invention, the mechanism of silencing encompasses the use of zinc finger proteins (ZFPs, otherwise known as zinc finger nucleases or ZFNs). A ZFP is a heterodimer in which each subunit contains a zinc finger domain and a FokI endonuclease domain. ZFPs constitute the largest individual family of transcriptional modulators known for higher organisms. In certain embodiments, the payload sequence comprises a DNA-binding domain made up of Cys2His2 zinc fingers fused to a KRAB repressor. In a preferred embodiment of the invention, the payload sequence comprises a zinc-finger-KRAB sequence.

### TALENs

In another embodiment of the invention, the mechanism of silencing encompasses the use of transcription activator-like effector nucleases (TALENs). TALENs comprise a non-specific DNA-cleaving nuclease fused to a DNA-binding domain that can be customised so that TALENs can target a sequence of interest to be silenced (Joung and Sander, 2013). In certain embodiments, the payload sequence comprises a TALEN sequence.

### Pharmaceutical Compositions

The invention provides a pharmaceutical composition comprising a nucleic acid molecule (for example, an AON, expression construct or vector), AAV particle or small molecule inhibitor of the invention.

Administration of an effective dose of the compositions may be by routes standard in the art including, but not limited to, intramuscular, parenteral, intravenous, oral, buccal, nasal, pulmonary or transdermal. Route(s) of administration may be chosen by those skilled in the art taking into account the infection and/or disease state being treated and the target cells/tissue(s) that are to express the nucleic acids.

For example, when the therapeutic agent is an AAV particle, any physical method that will transport the AAV vector into the target tissue of an animal may be used. Simply resuspending an AAV in phosphate buffered saline has been demonstrated to be sufficient to provide a vehicle useful for muscle tissue expression, and there are no known restrictions on the carriers or other components that can be co-administered with the AAV (although compositions that degrade DNA should be avoided in the normal manner with AAV). Capsid proteins of an AAV may be modified so that the AAV is targeted to a particular target tissue of interest such as muscle. See, for example, WO 02/053703, the disclosure of which is incorporated by reference herein.

The composition may additionally comprise a pharmaceutically acceptable carrier, diluent, excipient, adjuvant, buffer, stabilizer, and/or other materials well known to those skilled in the art. Such materials should be non-toxic and should not interfere with the efficacy of the active ingredient. The precise nature of the carrier or other material may be determined by the skilled person according to the route of administration.

For purposes of intramuscular injection, solutions in an adjuvant such as sesame or peanut oil or in aqueous propylene glycol can be employed, as well as sterile aqueous solutions. Such aqueous solutions can be buffered, if desired, and the liquid diluent first rendered isotonic with saline or glucose. For example, solutions of AAV as a free acid (DNA contains acidic phosphate groups) or a pharmacologically acceptable salt can be prepared in water suitably mixed with a surfactant such as hydroxpropylcellulose. A dispersion of AAV can also be prepared in glycerol, liquid polyethylene glycols and mixtures thereof and in oils. In all cases the form must be sterile and preserved against the contaminating actions of microorganisms such as bacteria and fungi. The prevention of the action of microorganisms can be brought about by various antibacterial and antifungal agents, for example, parabens, chlorobutanol, phenol, sorbic acid, thimerosal and the like.

When the therapeutic is delivered by an AAV vector, sterile injectable solutions are prepared by incorporating AAV in the required amount in the appropriate solvent with various other ingredients enumerated above, as required, followed by filter sterilization. Generally, dispersions are prepared by incorporating the sterilized active ingredient into a sterile vehicle which contains the basic dispersion medium and the required other ingredients from those enumerated above. In the case of sterile powders for the preparation of sterile injectable solutions, the preferred methods of preparation are vacuum drying and the freeze drying technique that yield a powder of the active ingredient plus any additional desired ingredient from the previously sterile-filtered solution thereof

For delayed-release, the vector may be included in a pharmaceutical composition that is formulated for slow release, such as in microcapsules formed from biocompatible polymers or in liposomal carrier systems according to methods known in the art. Prolonged absorption of the injectable compositions can also be brought about by use of agents delaying absorption, for example, aluminium monostearate and gelatin.

Combination therapies are also contemplated by the invention. Combination as used herein includes both simultaneous treatment or sequential treatments. Combinations of methods of the invention with standard medical treatments (e.g., non-steroidal anti-inflammatory drugs, NSAIDs) are specifically contemplated, as are combinations with novel therapies. Suitable combinations of therapeutics include, but are not limited to, therapeutics that target the necroptosis pathway. In particular, combinations of inhibitors of Ripk3, Ripk1, Mlk1 and/or Dux4 and epigenetic regulators may be used. For example, Dux4 inhibitors that may be combined with the inhibitors of the present invention include AONs and small molecule inhibitors of Dux4.

Dosages and dosage regimes can be determined within the normal skill of the medical practitioner responsible for the administration of the composition. The dosage of the active agent(s) may vary, depending on the reason for use, the individual subject, and the mode of administration. The dosage may be adjusted based on the subject's weight, the age and health of the subject, and tolerance for the compound(s) or composition.

### Host Cells

The invention additionally provides a host cell comprising a nucleic acid molecule (for example, an AON, expression construct or vector) or AAV viral particle disclosed herein. Any suitable host cell can be used to produce the nucleic acid molecule disclosed herein. In general, such cells will be transfected mammalian cells, but other cell types, e.g. insect cells, can also be used. In terms of mammalian cell production systems, HEK293 and HEK293T are preferred for AAV vectors. BHK or CHO cells may also be used.

### Methods of Therapy and Medical Uses

The therapeutic agents of the invention are useful for the treatment or prevention of FSHD. For example, nucleic acid molecules, small molecule inhibitor or compositions of the invention can be used to silence the expression or activity of genes involved in FSHD via the necroptosis pathway.

The therapeutic agents disclosed herein may be administered to patients who have been diagnosed with FSHD. Means for diagnosing FSHD are known in the art, and include southern blot, molecular combing to determine the size of the D4Z4 array, 4aA/4qB haplotyping, and/or a methylation assay to, for example, identify whether there is hypomethylation of the D4Z4 repeat.

FSHD causes muscle atrophy. Specifically, symptoms of FSHD include muscle weakness, for example abdominal muscle weakness, hip weakness, lower leg weakness (e.g., peroneal muscle weakness), shoulder weakness (e.g., scapular winging) and/or facial weakness, lordosis, scoliosis, dysphagia, foot drop, inflammation of the muscles, or any combination thereof. Non muscular symptoms include retinal vasculopathy, hearing loss and respiratory involvement. The present invention may therefore be used to treat or alleviate any combination of the above-mentioned symptoms. In preferred embodiments, the present invention may be used to treat or alleviate muscular symptoms.

Also provided is a method for treating facioscapulohumeral dystrophy (FSHD), the method comprising administering an inhibitor of the necroptosis pathway to a patient in need thereof. Preferably, the inhibitor will inhibit the expression or activity of at least one of RIPK3, RIPK1 and MLKL. For example, the inhibitor may be a small molecule inhibitor, an antisense oligonucleotide, an expression construct or a vector encoding said antisense oligonucleotide, as described above. The inhibitors may be administered to the patient in the form of a pharmaceutical composition.

Also provided is the use of the above-mentioned nucleic acid molecule, small molecule inhibitor or composition in a method for treating or preventing FSHD in a patient in need thereof.

Also provided is the use of a nucleic acid molecule, small molecule inhibitor or composition in the manufacture of a medicament for the treatment or prevention of FSHD.

One skilled in the art will recognize that the amount of a therapeutic to be administered will be an amount that is sufficient to induce amelioration of unwanted disease symptoms (such as FSHD symptoms). Such an amount may vary inter alia depending on such factors as the gender, age, weight, overall physical condition of the patient, etc. and may be determined on a case by case basis. The amount may also vary according to other components of a treatment protocol (e.g. administration of other medicaments, etc.). If a viral-based delivery is chosen, suitable doses will depend on different factors such as the virus that is employed, the route of delivery (intramuscular, intravenous, intra-arterial or other).

Further, those of skill in the art will recognize that, while disease symptoms may be completely alleviated by the treatments described herein, this need not be the case. Even a partial or intermittent relief of symptoms may be of great benefit to the recipient. Thus, as used herein, “therapeutically effective amount” means an amount of a nucleic acid set forth herein that, when administered to a mammal, is effective in producing the desired therapeutic effect.

### Kits

The present invention also includes kits, e.g., comprising one or more of the described therapeutic agents instructions for their use for treating FSHD. The instructions may include directions for using the therapeutic agents in vitro, in vivo or ex vivo. Typically, the kit will have a compartment containing the therapeutic agent. The therapeutic agent may be in a lyophilized form, liquid form, or other form amendable to being included in a kit. The kit may also contain additional elements needed to practice the method described on the instructions in the kit, such a sterilized solution for reconstituting a lyophilized powder, additional agents for combining with the therapeutic agent prior to administering to a patient, and tools that aid in administering a therapeutic agent to a patient.

### EXAMPLES

The following examples are provided to further illustrate the compositions and methods of the present invention. These examples are illustrative only and are not intended to limit the scope of the invention in any way.

### Example 1: Materials and Methods

### I. Animals Housing and Crosses.

FLExDUX4 (B6(Cg)-Gt(ROSA)26Sortm1.1(DUX4*)Plj/J) and HSA-Cre (B6. Cg-Tg(ACTA1-cre/Esr1)2Kesr/J) mice were purchased from the Jackson Laboratory (#001801, #028710, #025750). RipK3−KO mice (C57BL/6 Ripk3+/−) were generated after crossing back the Mdx/RipK3^{(-/-)} mice to remove the mdx mutation (Morgan, 2018). These 3 strains were crossed to generate Cre^{(+/-)}RipK3^{(-/-)} and FLExDUX4^{(+/+)}RipK3^{(-/-)} mice, and bred together to generate progeny Cre^{(+/-)}FLExDUX4^{(+/-)}RipK3^{(-/-)}/Cre^{(-/-)}FLExDUX4^{(+/-)}RipK3^{(-/-)} (CDR/Cre+ and CDR/Cre− respectively) used in the experiments.

Three-week-old animals were weighted 3 times a week, after weaning. The tamoxifen (MP Biomedicals) injections were realized according to Jones et al (Jones, 2018). Six-week-old mice were injected (IP) with Tamoxifen (TMX) on two consecutive days for a final concentration of 10 mg/kg. Following TMX injection, animals were daily weighed, and sacrificed after a week. Muscles were harvested and frozen in liquid nitrogen or liquid-nitrogen-cooled isopentane for further analysis.

### II. Cell Culture, Viability, Cytotoxicity and Caspase Activation.

Inducible iC2C12-Dux4 (Bosnakovski, 2008) were cultured in DMEM high glucose with GlutaMAX and no Sodium Pyruvate (Gibco), 20% fetal bovine serum, and 800 μg/mL G418 (Gibco). For myoblast experiments, iC2C12-Dux4 were seeded into white 96-well plates (3000 cells per well) in growth medium. After 24 h, DUX4 expression was induced by adding doxycycline (Sigma-Aldrich) at a final concentration of 50 to 1000 ng/mL. For myotubes experiments, iC2C12-Dux4 were seeded into white 96-well plates (1500 cells per well) in growth medium. After 48 h, cells were differentiated with DMEM, 2% horse serum and 10 μg/mL insulin. DUX4 expression was induced after 4 days of differentiation to not disturb myotube formation (Bosnakovski, 2008). Inhibition of apoptosis and/or necroptosis was simultaneously realized to DUX4 induction for 24 h using 20 μM of pan-caspase inhibitor Z-VAD.fmk (Merk Chemicals), 30 μM Necrostatin-1, 3 μM GSK872, 0.2 Cyclosporin A (Cambridge Bioscience). Cell survival was determined by CellTiter-Glo luminescent cell viability assay (Promega), membrane permeability by CytoTox-Glo cytotoxicity assay (Promega) and caspase activation by Caspase-Glo 3/7 Assay (Promega) according to the manufacturer's instructions. Luminescence was read by a microplate reader Tecan.

III. RNA Extraction, qPCR

For muscles, cryopreserved tissues were transferred in tube containing 1.4 mm ceramic beads (FastPrep, MP biomedicals, UK) plus 1 mL of Trizol (Thermo Fisher Scientific, Paisley, UK) and shaken at 10000 rpm for 40 s. Total RNAs from cells or tissue were extracted using Trizol (Thermo Fisher) according the manufacturer's protocol. The quantity of RNA was determined using a nanodrop ND-1000 spectrophotometer (Thermo Scientific, Wilmington, Del., USA). Reverse transcription was performed on 1 μg of total RNA in a 10 μL final volume (Roche Transcriptor First Strand cDNA Synthesis kit). Quantitative PCR (qPCR) was designed according to the MIQE standards. qPCRs were performed on a LightCycler 480 Real-Time PCR System (Roche) in a final volume of 9 with 0.2 μL of RT product, 0.4 μM each of forward and reverse primers (see Tables 2 and 3), and 4.5 μL of SYBRGreen Mastermix (Roche).

TABLE 4
RT-PCR forwards primers

| | | SEQ ID |
|---|---|---|
| Gene | Forward primer | NO |
| Dux4-all | GGCCCGGTGAGAGACTCCACA | 36 |
| Hprt1 | TGATCAGTCAACGGGGGACA | 37 |
| mDux-bl | GCATCTCTGAGTCTCAAATTATGACTTG | 38 |
| mGapdh | CACCCACCCCAGCAAGGA | 39 |
| mMlkl | ATCAAAGTATTCAACAACCCC | 40 |
| mRipk1 | AGAAGAAGGGAACTATTCGC | 41 |
| mRipk3 | CGGGCACACCACAGAACAT | 42 |
| mSnx30 | ATTATGAGAAGTGCCTCATGG | 43 |
| mTm7sf4 | TCCTCCATGAACAAACAGTTCC | 44 |
| mWfdc3 | GGTAGCTGCAGGAGAGCACG | 45 |
| P0 | TGCTCGACATCACAGAGCAG | 46 |
| mPsma2 | AGAGCGCGGTTACAGCTTC | 47 |

TABLE 5
RT-PCT reverse primers

| | | SEQ ID |
|---|---|---|
| Gene | Reverse primer | NO |
| Dux4-all | CCAGGAGATGTAACTCTAATCCAGGTTTGC | 48 |
| Hprt1 | TCCAACACTTCGAGAGGTCC | 49 |
| mDux-bl | GCGTTCTGCTCCTTCTAGCTTCT | 50 |
| mGapdh | ATGGGGGTCTGGGATGGAAA | 51 |
| mMlkl | GCAAATCCCAAATATACGCAA | 52 |
| mRipk1 | TTCTATGGCCTCCACGAT | 53 |
| mRipk3 | GTAGCACATCCCCAGCACCAC | 54 |
| mSnx30 | GACATTCTGGTTCAGGGTTC | 55 |
| mTm7sf4 | AATCATGGACGACTCCTTGGG | 56 |
| mWfdc3 | CTGGGGACAGGATTCGTCTC | 57 |
| P0 | GATCTGCAGACACACACTGG | 58 |
| mPsma2 | CTCCACCTTGTGAACACTCCTT | 59 |

IV. CD68 and IgG uptake staining

The analysis of CD68 positive cells infiltration and IgG uptake was performed on 10 μm transverse cryosections from the quadriceps muscle. Sections stored at −80° C. were dried at room temperature for 30 min and fixed with 4% paraformaldehyde for 10 min. Following three washes with PBS, staining areas were delimited using a Dako pen. Blocking of unspecific sites and permeabilization were achieved by incubating samples in 20% Fetal Bovine Serum, 0.5% Tween 20, 0.5% Triton X100 and 5% BSA. Primary antibodies were diluted in 1% FBS and incubated 2 hrs at room temperature (IgG uptake analysis) or overnight at 4° C. (CD68 positive cells infiltration analysis). Secondary antibodies diluted I 1% FBS were incubated for 1 hr at room temperature and sections were stained for nuclei with Hoechst for 15 min. For immunofluorescence analysis, rat IgG2A antibody to CD68 (clone FA-11, Biolegend #137001, 1/50), mouse antibody to Laminin (Dako #Z0097, 1/400), goat anti-mouse IgG (Biotin-XX, Invitrogen, 1/400), mouse anti-rat IgG2A eFluor 615 Texas Red (1/400), goat anti-rabbit Alexa Fluor 488 (1/400), Streptavidin Protein, Dy Light 488 (1/400) were used.

Pictures were acquired using ThermoScientific™ Invitrogen™ EVOS™ FL Auto 2 Imaging System and 20× objectives. Sections were entirely scanned and pictures were analysed using ImageJ software.

### Example 2: DUX4 Expression Causes Ripk1-Mediated Necroptosis in iC2C12-DUX4 Myoblasts

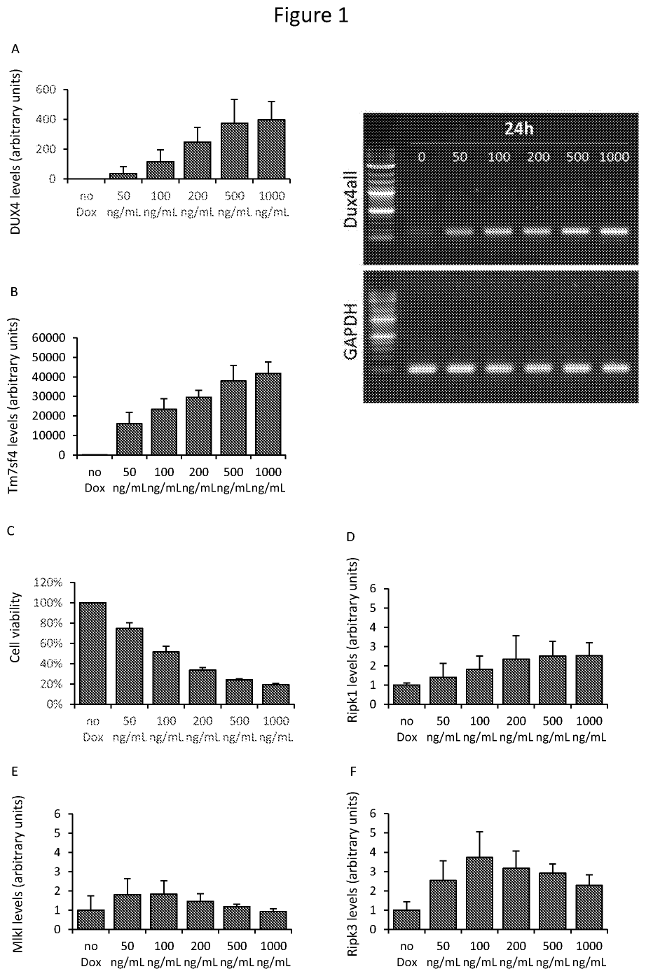

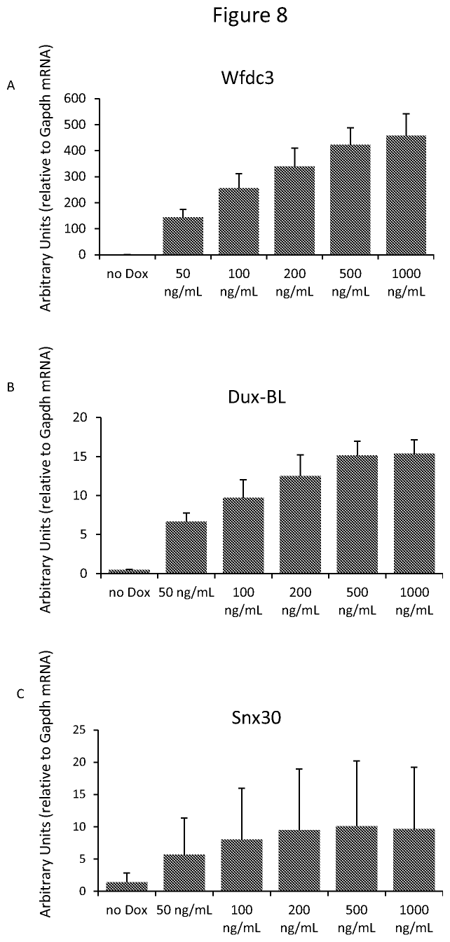

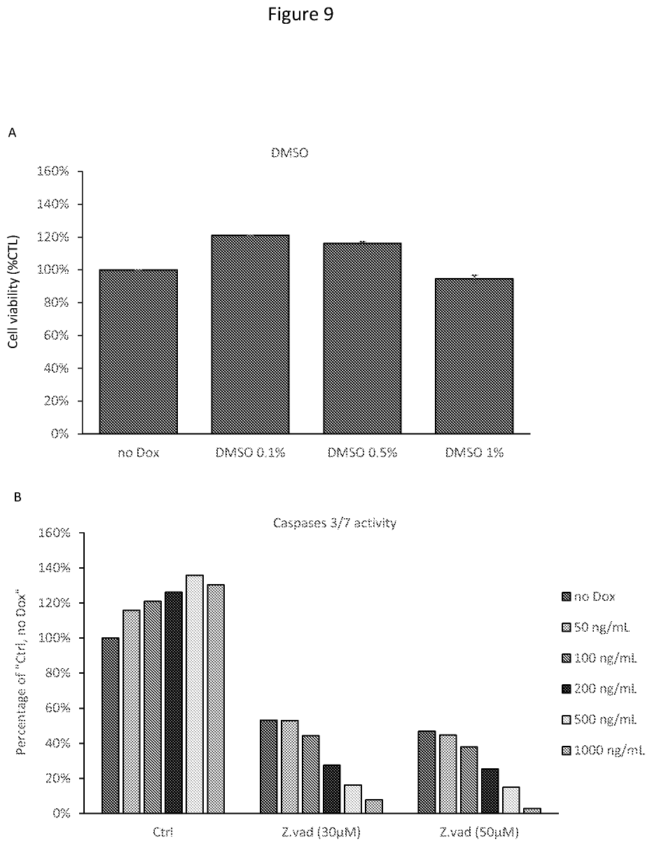

To investigate the role of necroptosis in DUX4 toxicity, we used the iC2C12-DUX4 cells that carry a doxycycline (dox)-inducible DUX4 transgene (Bosnakovski, 2008). In the presence of dox, DUX4 is expressed in a dose dependent manner (FIG. 1A), and consequently genes downstream of DUX4 are transcribed (FIG. 1B and FIG. 8), and cell viability dramatically decreased (19.3%±1.2% of viable cells at 1000 ng/ml of dox) (FIG. 1 C). Non-induced cells expressed low levels of the 3 major genes involved in necroptosis Ripk1, RipK3 and Mlk1, but after DUX4 induction, expression increased up to 2.52±0.67 times for Ripk1, 1.83±0.69 for Mlk1 and 3.7±1.34 for Ripk3 (FIG. 1 D, E, F respectively).

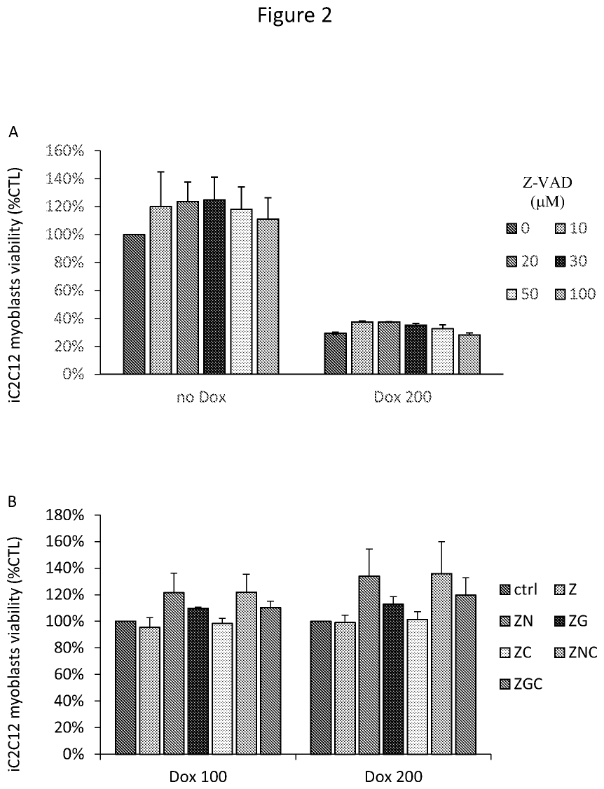

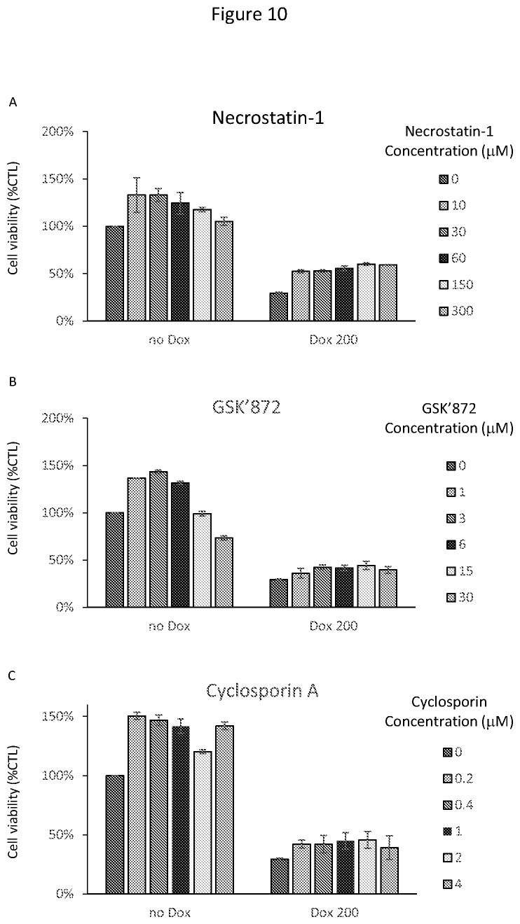

The inventors next asked whether increased necroptosis gene levels have any effect on cell viability. First they investigated the role of the caspases by cultivating the iC2C12-DUX4 cells in the presence of 200 ng/ml of dox (dose leading to an important expression of DUX4 with a cell viability of 35%, FIGS. 1A and C) and with different doses of Z-VAD, a pan-caspase inhibitor. No change in cell survival was observed with and without Z-VAD (FIG. 2A), indicating that caspases are not involved in the death of the iC2C12-DUX4 cells. It is worth noting that the addition of Z-VAD induced a non-specific increase of cell survival that is due to the presence of DMSO (Fig. (A). It was confirmed that caspase 3/7 activity increased after DUX4 expression and was correctly inhibited in the presence of Z-VAD (FIG. S2B). Next the role of necroptosis in DUX4 mediated cell death was investigated by the addition of either necrostatin-1 (Ripk1 inhibitor) or GSK'872 (Ripk3 inhibitor) alone or in combination. A dose response was first performed (FIG. 10) allowing the determination of the optimal dose for each compound: 30 μM for necrostatin-1 and 3 μM for GSK'872 (FIG. 10). The iC2C12-DUX4 viability was assessed for 2 different doses of dox and the results are expressed as the percentage of cells alive in the presence of the different compounds compared to the condition without them. Z-VAD was always added to eliminate any bias linked to caspases 3/7 activation/synergic effect. The only condition that induced a cell rescue was when necrostatin-1 was present. In the presence of 100 ng/ml dox, the 2 combinations Z-VAD/necrostatin-1 and Z-VAD/necrostatin-1/cyclosporine A lead to an increase of cell survival by 22%±15 (FIG. 2B). Similar results were obtained when the dox concentration was 200 ng/ml (increase of cells survival by 34%±20 when necrostatin-1 is present), thus demonstrating the role of Ripk1-mediated necroptosis in DUX4-mediated cell death in iC2C12-DUX4 myoblasts.

### Example 3: DUX4 Expression Causes Ripk3-Mediated Necroptosis in iC2C12-DUX4 Myotubes

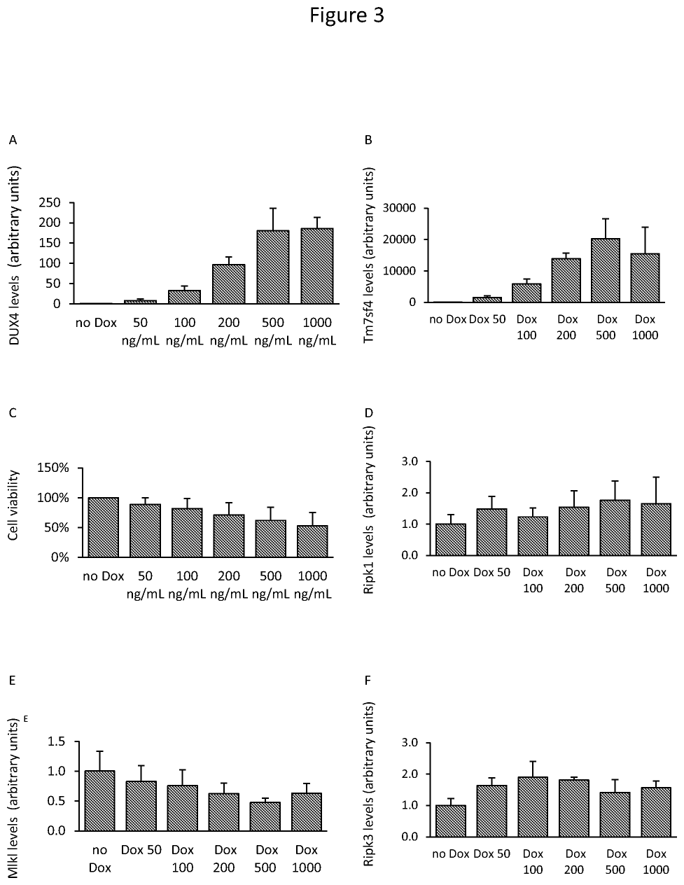

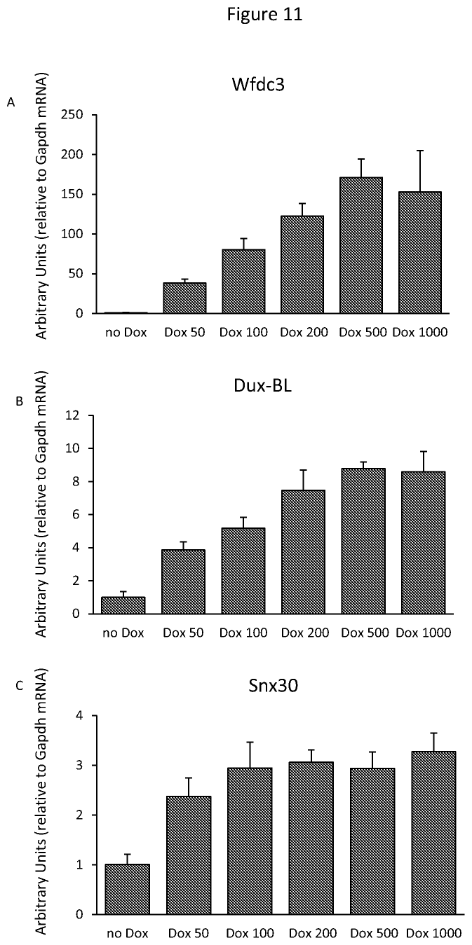

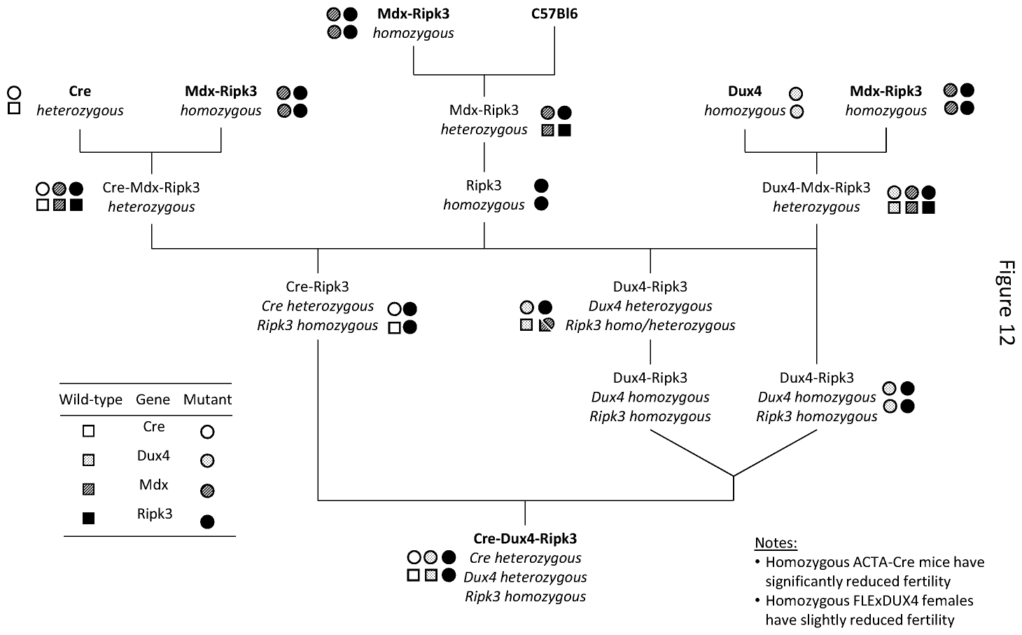

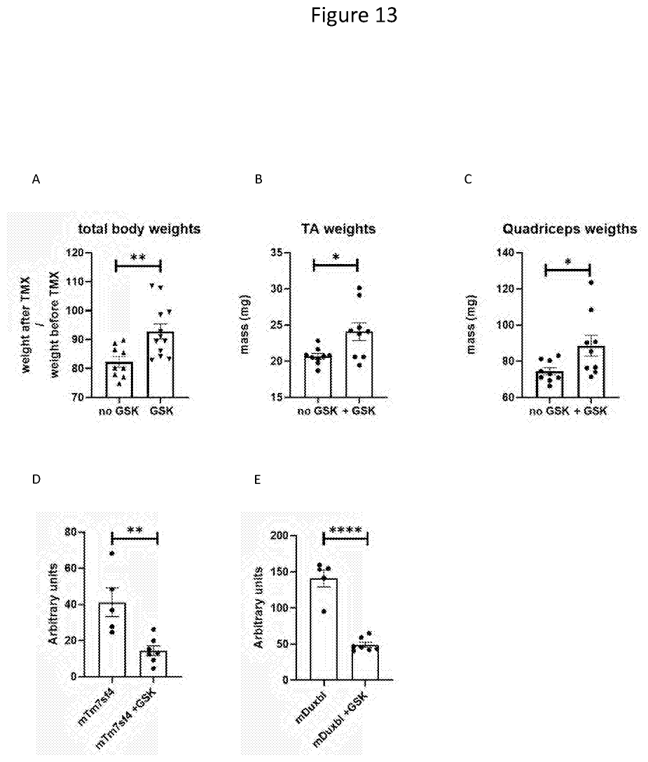

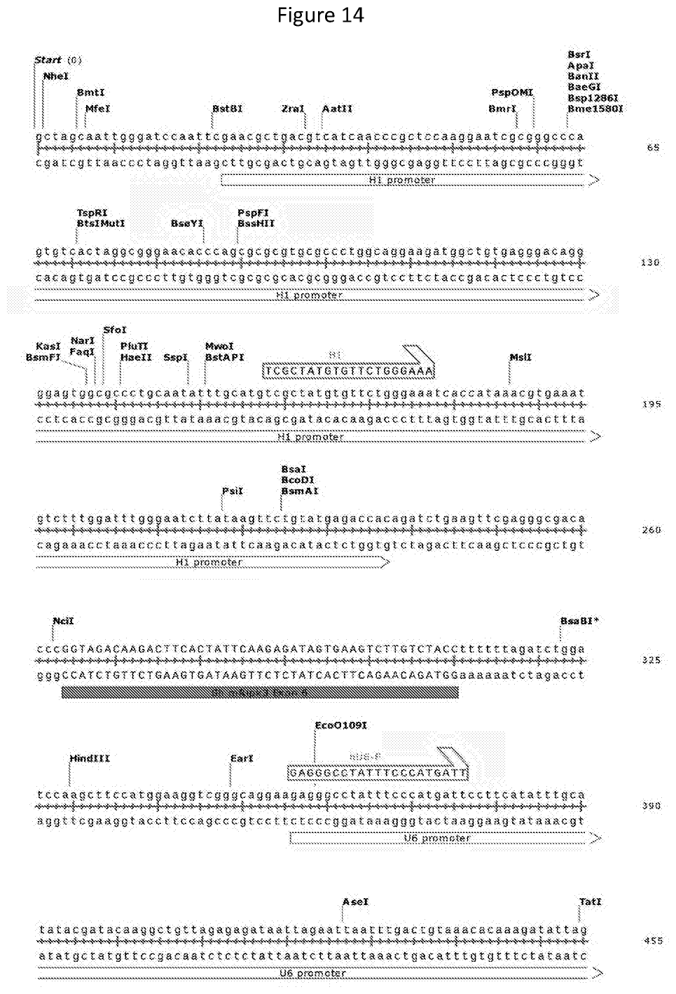

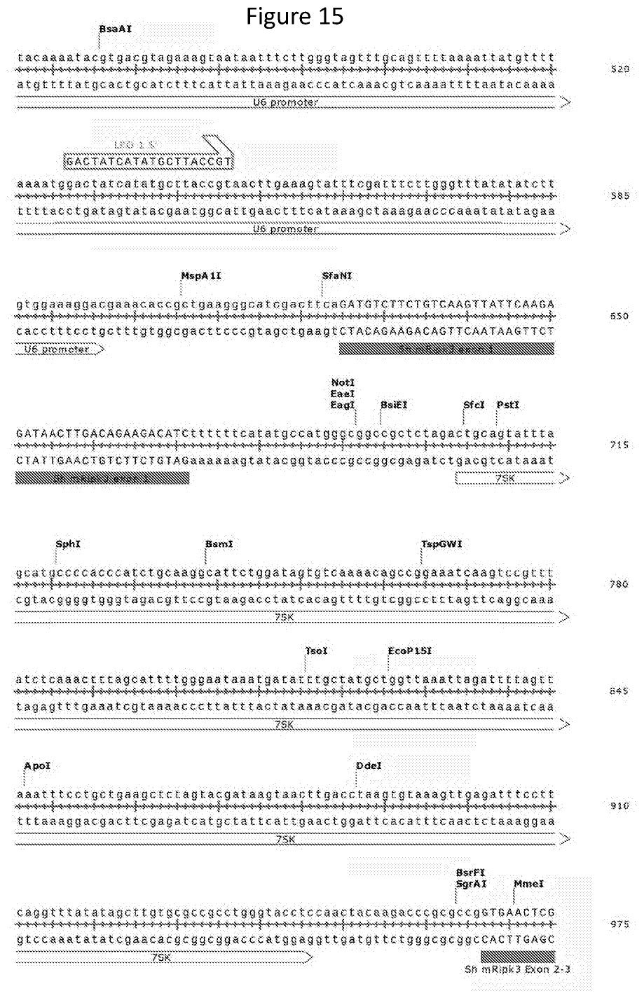

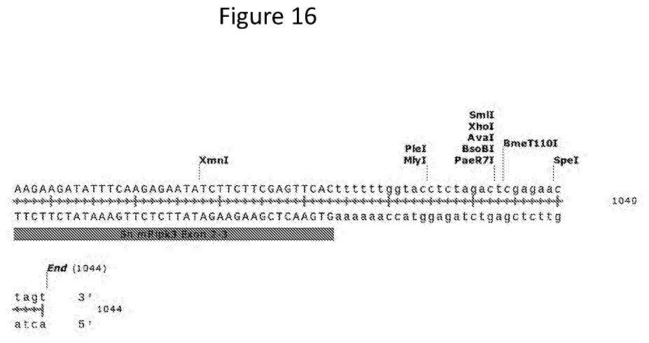

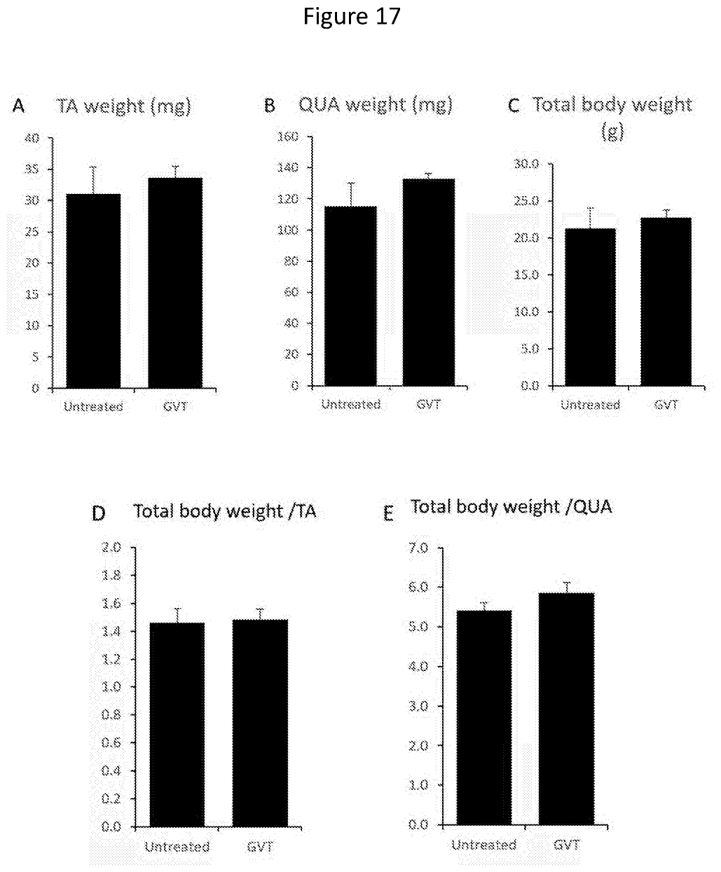

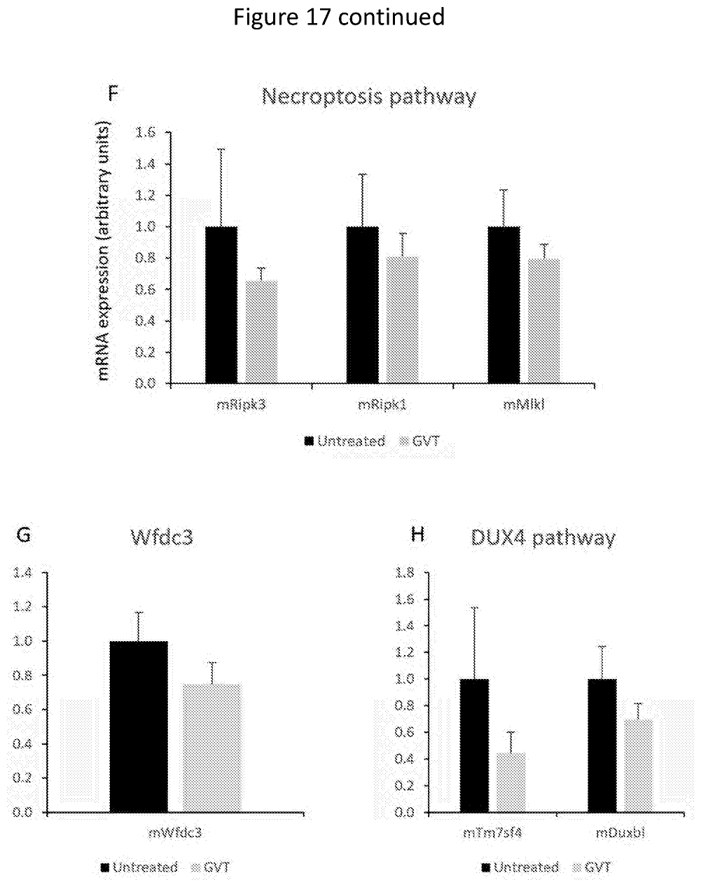

The inventors next investigated DUX4-mediated toxicity in iC2C12-DUX4 myotubes. An increase in DUX4 mRNA was observed after addition of dox in dose dependent manner (FIG. 3A), associated with an increase of several genes downstream of DUX4 including Tm7sf4 (FIG. 3B), Wfdc3, Dux-Bl and Snx30 (FIG. 11) and a decrease in cell viability (FIG. 3C). The expression of Ripk1 was not affected by DUX4 expression, while Mlk1 decreased when dox concentrations increased (FIGS. 3D and E). Ripk3 level was increased up to 1.9±0.5 fold (FIG. 3F).

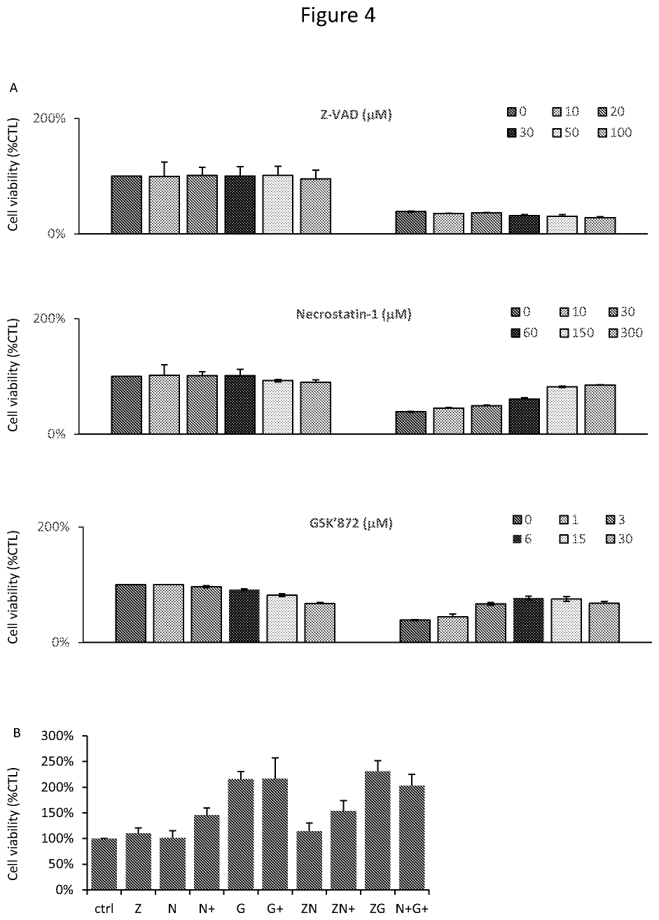

The effects of the different inhibitors on iC2C12-DUX4 myotubes were investigated. Again, the results are expressed as the percentage of live cells in the presence of the different compounds compared to the control condition without. No effect of Z-VAD were observed arguing against a major role of caspases in DUX4-mediated toxicity (FIG. 4A). When the cells were cultivated with necrostatin-1 in the presence of dox (1000 ng/ml), necrostatin-1 concentrations below 60 μM conferred a protection against cell death (up to 60%±6 of viable cells in presence of 60 μM necrostatin-1 compared to 39%±1 without, FIG. 4A), necrostatin-1 concentrations of 150 or 300 μM leading to up to 85%±2 viable cells (FIG. 4A). When the iC2C12-DUX4 myotubes were incubated with GSK'872, a massive cell death rescue was observed at low concentrations (up to 67%±2 of viable cells in presence of 3 μM GSK'872 compared to 39%±1 without, FIG. 4C). Higher GSK'872 concentrations slightly improved cell viability (up to 77%±4 of viable cells in presence of 6 μM GSK'872, FIG. 4C). These results demonstrated the role of necroptosis in DUX4 mediated myotube death. The different compounds were next added separately or together to iC2C12-DUX4 myotubes that were incubated with dox (1000 ng/ml). No modification of the cell viability was observed in the presence of 20 μM Z-VAD/30 μM necrostatin-1 but when necrostatin-1 concentration was increased to 90 μM, the combination 20 μM Z-VAD/90 μM necrostatin-1 lead to cell rescue (increase of cell survival by 54%±20 with necrostatin-1 compared to 10%±10 for Z-VAD alone). The best cell rescue was observed in the presence of GSK'872. Indeed, at low (2 μM) or high (4 μM) concentration, GSK'872 increased cell viability by 2.1 fold (FIG. 4D). The combination necrostastin-1/GSK'872 did not shown any additive or synergistic effect. These results demonstrated the important role of necroptosis in DUX4-mediated cell death. Ripk3 but also to a lesser extent Ripk1, participated in DUX4-mediated cell toxicity.

### Example 4: Necroptosis Participates in DUX4-Mediated Toxicity In Vivo

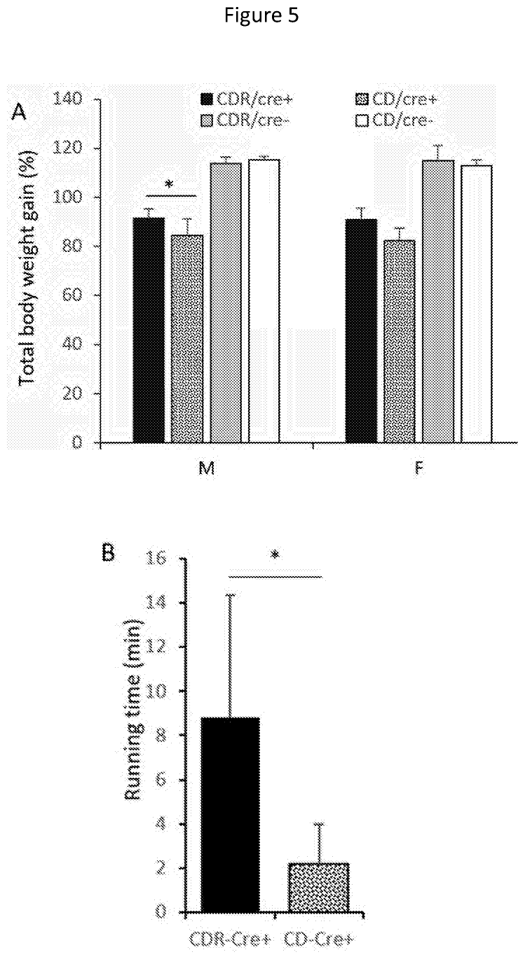

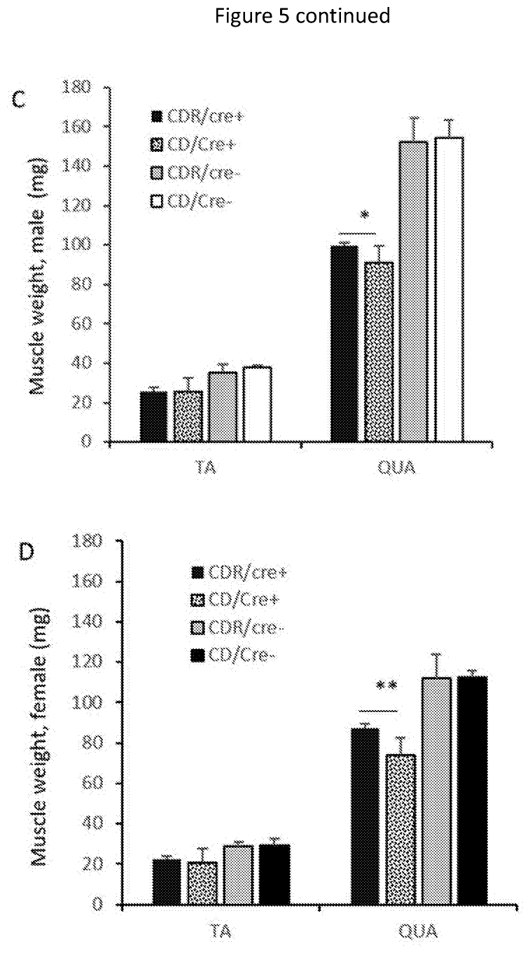

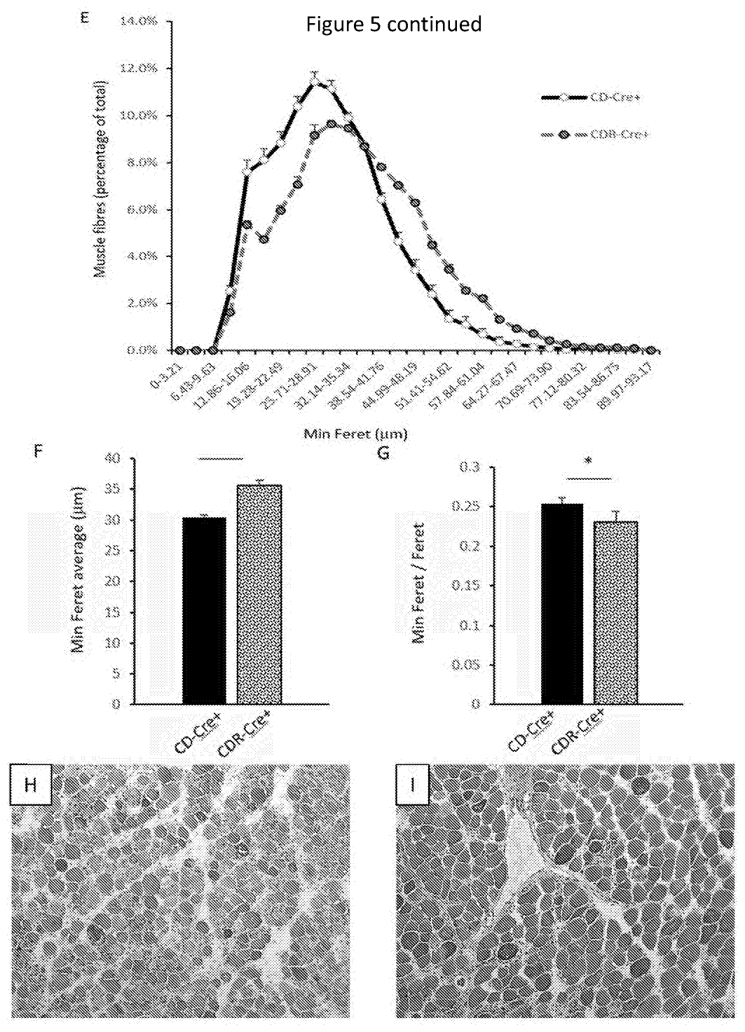

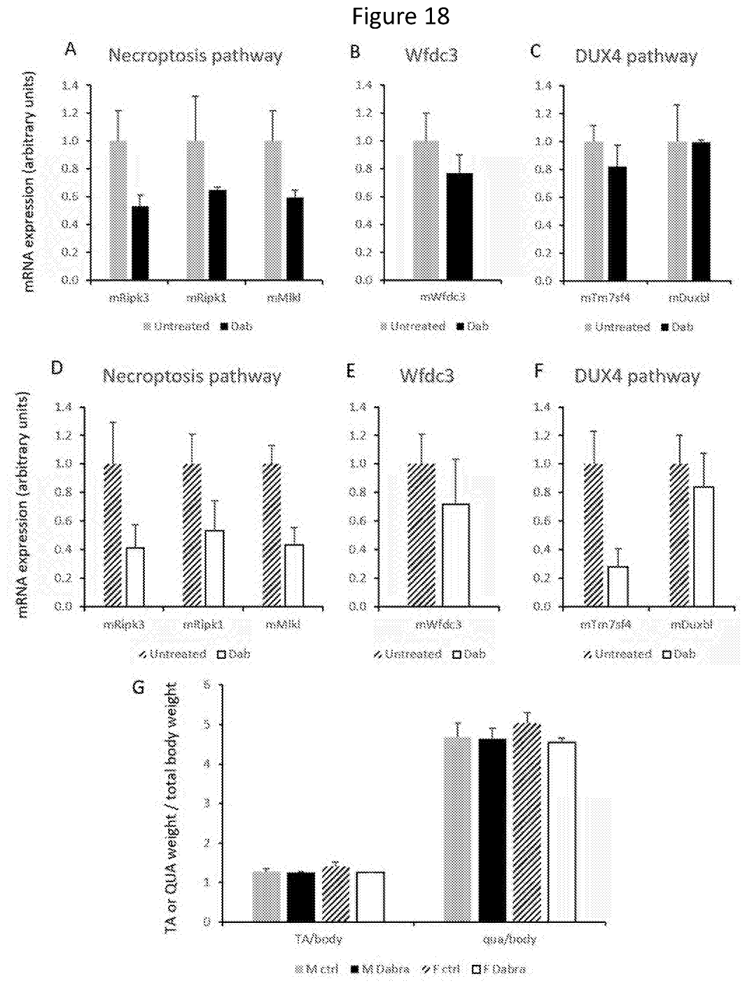

The experiments performed in cell culture suggested that necroptosis is a key element of DUX4-mediated toxicity. To evaluate the role of necroptosis in vivo, the cre-inducible DUX4 transgenic mouse model (FLExDUX4, here called CD) that conditionally expresses human DUX4 following tamoxifen injection (Jones, 2018) was used. This model was crossed with a Ripk3-deficient mouse model (Morgan, 2018), leading to the new transgenic CDR (DUX4−/+Rip−/−) mouse model (FIG. 12). After tamoxifen injection, total body weight was measured and FIG. 5A represents the variation of the total body weight gain (in percentage) from the beginning of treatment to animal death. In CD- and CDR-cre-negative animals, a 13-15% increase of the total body weight was observed in one 1 week (FIG. 5A) but in both CD- and CDR-cre-positive animals, a decrease in of the total body weight was seen. This decrease is less pronounced in the CDR animals (in males, 91.4%±3.9 for the CDR/cre+ and 84.6%±4.8 for the CD/Cre+, p=0.02; in females, 90.8%±6.7 for the CDR/cre+ and 82.5%±4.9 for the CD/Cre+, p=0.07). These results indicate the role of necroptosis in DUX4-mediated toxicity leading to weight loss.

FIG. 5B represents total running time measured on a 15° angled treadmill system using a treadmill exhaustion test. This test consisted of an acclimatisation period of 5 min on the treadmill (set to an angle of 15°), followed by 5 min at 5 m/min. The speed was then increased by 0.5 m every minute. Electric shock on the treadmill was removed and mice were encouraged to run by gently pushing them. Mice refusing to run for 10 sec were removed from the treadmill and the total running time was recorded.

The weights of the tibialis anterior (TA) and quadriceps (QUA) were analysed. CD- and CDR-cre-negative animals showed similar TA or QUA muscle weights (FIG. 5C), CD- and CDR-cre-positive animals showed also comparable weights of the TA (˜25 mg for the males and ˜20-22 mg for the females). However, QUA weights were higher in CDR-cre-positive than in CD-cre-positive animals (in males, 99.4 mg±6.6 for the CDR/cre+ and 90.9 mg±8.4 for the CD/Cre+, p=0.006; in females, 87.3 mg±6.2 for the CDR/cre+ and 73.8 mg±6 for the CD/Cre+, p=5.45E-05), showing that QUA weights are higher when RIPK3 is not expressed,

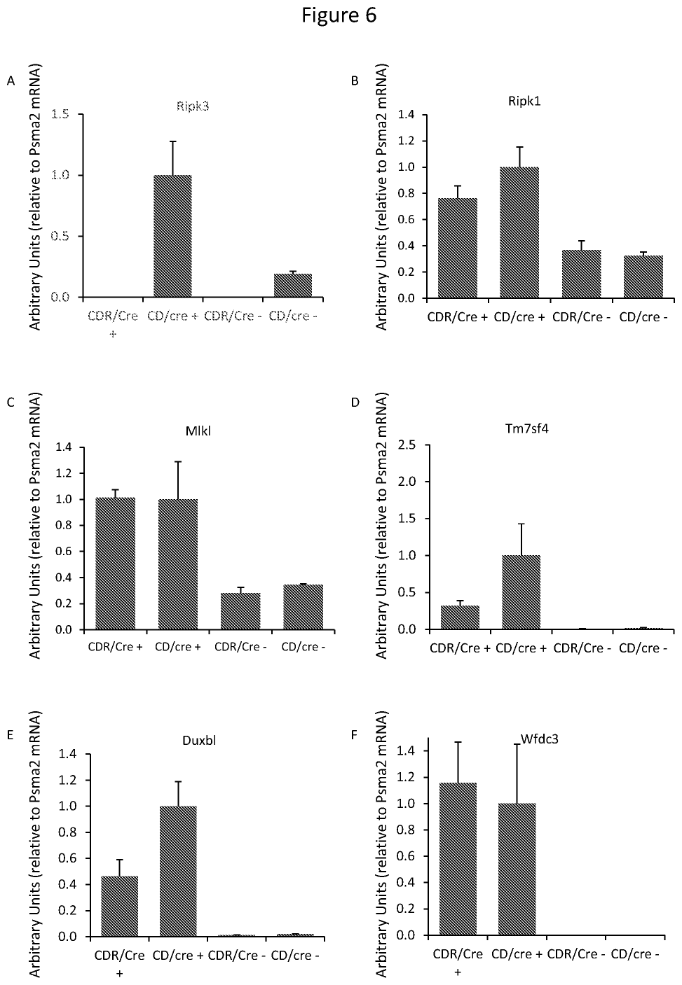

Next, the expression of Ripk1, Ripk3 and Mlk1 was investigated in the QUA only since variation in muscle weight was not observed in the TA of mice expressing or not Ripk3. No expression of Ripk3 was observed in the Ripk3-deficient mice (CDR-cre-positive or -negative, FIG. 6). Expression of Ripk1, Ripk3 and Mlk1 was higher in the CD-cre+ animals compared to the CD-cre-negative mice (3.1, 5.2 and 2.9 fold for Ripk1, Ripk3 and Mlk1 respectively, p=0.002; 0.01 and 0.03 respectively), thus showing that DUX4 expression induces necroptosis network activation. Interestingly, in the presence of Cre, the absence of RIPK3 did not modify the global mRNA levels of RIPK1 and MLKL (CDR/Cre+vs CD/Cre+, FIG. 6). However, the expression levels of 2 genes downstream of DUX4 were reduced in the CDR/Cre+ compared to the CD/Cre+ animals: by 3.1 fold (p=0.03) and 2.1 fold (p=0.005) for Tm7sf4 and mDuxb1 respectively. Expression levels of Wfdc3 remained unchanged.

### Example 5: RIPK3 Deficiency Ameliorates Muscle Phenotype of DUX4-Expressing Mice

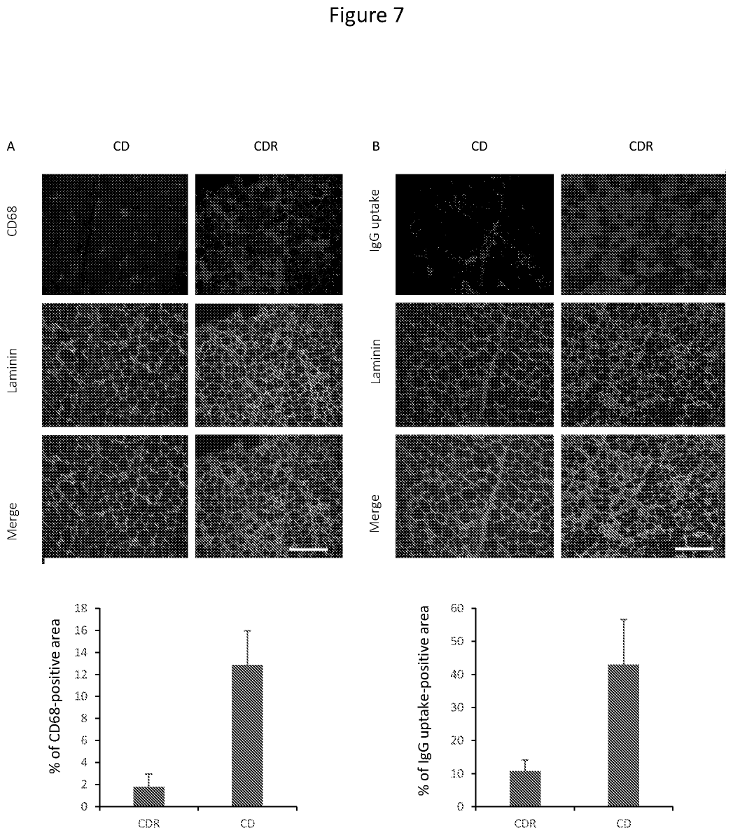

The inventors next questioned whether RIPK3 depletion ameliorates muscle phenotype after DUX4 expression. They investigated the presence of an inflammatory response and measured the muscle area infiltrated by macrophages using a CD68 antibody. Ripk3−/− CD mice had over 7 fold decrease (p=0.039) compared to RIPK3-competent CD mice (FIG. 7A), indicating a role of RIPK3 in inflammatory response to DUX4 expression. Myonecrosis was also investigated by IgG uptake labelling. The percentage of IgG-positive area was 4 fold decreased in the RIPK3-deficient mice (p=0.007) (FIG. 7B). These results show that RIPK3 deficiency suppresses inflammation-mediated muscle damage and ameliorates the muscle phenotype of DUX4-expressing mice and demonstrates the role of necroptosis in DUX4-mediated toxicity.

### Example 6: Design of shRNAs

The inventors designed expression constructs comprising sequences encoding shRNAs operably linked to RNA pol III promoters. An exemplary expression construct is shown in FIG. 14. The expression construct was cloned using restriction sites into an AAV vector for intramuscular or/and systemic injection into DUX4 mice.

For the shRNA constructs, the inventors used the sequences published in the Merienne, 2017 (SEQ ID NO: 61).

### Example 7: In Vivo Treatment with Galavit or Dabrafenib

Actal Cre-FLEx DUX4 mice were treated with either galavit or dabrafenib. Galavit (4.5 mg/kg) was injected intraperitoneally daily for 3 consecutive days to 7-8 week old males. Tamoxifen was intraperitoneally injected at day 0 and 1 (10 mg/kg). Mice were sacrificed 7 days after the 1st injection. Dabrafenib (30 mg/kg) was administered by oral (p.o.) gavage to 4-5 week old animals 5 times/week for 4 weeks. Tamoxifen was intraperitoneally injected weakly (2 mg/kg).

The effect, and lack thereof, of the weight of tibialis anterior (TA) and quadriceps (QUA) expressed as a ratio to total body weight between control animals and animals treated with galavit or dabrafenib is shown in FIG. 17D-E and FIG. 18G.

The expression of necroptotic genes (Ripk1, Ripk3 and Mlk1) and DUX4-network genes (Tm7sf4, mDuxb1 and Wfdc3) was investigated in animals treated with either galavit or dabrafenib. Treatment with either galavit or dabrafenib resulted in the decreased mean expression of almost all of these genes, relative to expression in untreated control animals (FIG. 17F-H and FIG. 18A-F).

### REFERENCES

van Deutekom J C, Wijmenga C, van Tienhoven E A, Gruter A M, Hewitt J E, Padberg G W, van Ommen G J, Hofker M R, Frants R R. FSHD associated DNA rearrangements are due to deletions of integral copies of a 3.2 kb tandemly repeated unit. Hum Mol Genet 1993; 2(12):2037-2042.
Wijmenga C, Hewitt J E, Sandkuijl L A, Clark L N, Wright T J, Dauwerse H G, Gruter A M, Hofker M R, Moerer P, Williamson R, et al. Chromosome 4q DNA rearrangements associated with facioscapulohumeral muscular dystrophy. Nat Genet 1992; 2(1):26-30.
Lemmers R J, Tawil R, Petek L M, Balog J, Block G J, Santen G W, Amell A M, van der Vliet P J, Almomani R, Straasheijm K R, Krom Y D, Klooster R, Sun Y, den Dunnen J T, Helmer Q, Donlin-Smith C M, Padberg G W, van Engelen B G, de GreefJC, Aartsma-Rus A M, Frants R R, de Visser M, Desnuelle C, Sacconi S, Filippova G N, Bakker B, Bamshad M J, Tapscott S J, Miller D G, van der Maarel S M. Digenic inheritance of an SMCHD1 mutation and an FSHD-permissive D4Z4 allele causes facioscapulohumeral muscular dystrophy type 2. Nat Genet 2012; 44(12):1370-1374.
Snider L, Asawachaicharn A, Tyler A E, Geng L N, Petek L M, Maves L, Miller D G, Lemmers RJLF, Winokur S T, Tawil R, van der Maarel SrM, Filippova G N, Tapscott S J. RNA transcripts, miRNA-sized fragments and proteins produced from D4Z4 units: new candidates for the pathophysiology of facioscapulohumeral dystrophy. Human molecular genetics 2009; 18(13):2414-2430.
Ferreboeuf M, Mariot V, Furling D, Butler-Browne G, Mouly V, Dumonceaux J. Nuclear protein spreading: implication for pathophysiology of neuromuscular diseases. Hum Mol Genet 2014; 23(15):4125-4133.
Snider L, Geng L N, Lemmers R J, Kyba M, Ware C B, Nelson A M, Tawil R, Filippova G N, van der Maarel S M, Tapscott S J, Miller D G. Facioscapulohumeral dystrophy: incomplete suppression of a retrotransposed gene. PLoS Genet 2010; 6(10):e1001181.
Ferreboeuf M, Mariot V, Bessieres B, Vasiljevic A, Attie-Bitach T, Collardeau S, Morere J, Roche S, Magdinier F, Robin-Ducellier J, Rameau P, Whalen S, Desnuelle C, Sacconi S, Mouly V, Butler-Browne G, Dumonceaux J. DUX4 and DUX4 downstream target genes are expressed in fetal FSHD muscles. Hum Mol Genet 2014; 23(1):171-181.
DeSimone A M, Pakula A, Lek A, Emerson C P, Jr. Facioscapulohumeral Muscular Dystrophy. Compr Physiol 2017; 7(4):1229-1279.
Bosnakovski D, Xu Z, Gang E J, Galindo C L, Liu M, Simsek T, Garner H R, Agha-Mohammadi S, Tassin A, Coppee F, Belayew A, Perlingeiro R R, Kyba M. An isogenetic myoblast expression screen identifies DUX4-mediated FSHD-associated molecular pathologies. Embo J 2008; 27(20):2766-2779.
Rickard A M, Petek L M, Miller D G. Endogenous DUX4 expression in FSHD myotubes is sufficient to cause cell death and disrupts RNA splicing and cell migration pathways. Hum Mol Genet 2015; 24(20):5901-5914.
Block G J, Narayanan D, Amell A M, Petek L M, Davidson K C, Bird T D, Tawil R, Moon R T, Miller D G. Wnt/beta-catenin signaling suppresses DUX4 expression and prevents apoptosis of FSHD muscle cells. Hum Mol Genet 2013; 22(23):390-396.
Wallace L M, Garwick S E, Mei W, Belayew A, Coppee F, Ladner K J, Guttridge D, Yang J, Harper S Q. DUX4, a candidate gene for facioscapulohumeral muscular dystrophy, causes p53-dependent myopathy in vivo. Ann Neurol 2011; 69(3):540-552.
Dandapat A, Bosnakovski D, Hartweck L, Arpke R, Baltgalvis K, Vang D, Baik J, Darabi R, Perlingeiro R, Hamra F, Gupta K, Lowe D, Kyba M. Dominant lethal pathologies in male mice engineered to contain an X-linked DUX4 transgene. Cell Rep 2014; 8(5):1484-1496. doi: 1410.1016/j.celrep.2014.1407.1056. Epub 214 August 1428.
Lek A, Zhang Y, Woodman K G, Huang S, DeSimone A M, Cohen J, Ho V, Conner J, Mead L, Kodani A, Pakula A, Sanjana N, King O D, Jones P L, Wagner K R, Lek M, Kunkel L M. Applying genome-wide CRISPR-Cas9 screens for therapeutic discovery in facioscapulohumeral muscular dystrophy. Sci Transl Med 2020; 12(536).
McQuade T, Cho Y, Chan F K. Positive and negative phosphorylation regulates RIP1- and RIP3-induced programmed necrosis. Biochem J 2013; 456(3):409-415.
Silke J, Rickard J A, Gerlic M. The diverse role of RIP kinases in necroptosis and inflammation. Nat Immunol 2015; 16(7):689-697.
Jones T, Jones P L. A cre-inducible DUX4 transgenic mouse model for investigating facioscapulohumeral muscular dystrophy. PLoS One 2018; 13(2):e0192657.
Morgan J E, Prola A, Mariot V, Pini V, Meng J, Hourde C, Dumonceaux J, Conti F, Relaix F, Authier F J, Tiret L, Muntoni F, Bencze M. Necroptosis mediates myofibre death in dystrophin-deficient mice. Nat Commun 2018; 9(1):3655.
Kowaljow V, Marcowycz A, Ansseau En, Conde C B, Sauvage Sb, MattÃ©otti C, Arias C, Corona E D, NuA±ez NsG, Leo O, Wattiez R, Figlewicz D, Laoudj-Chenivesse D,
Belayew A, CoppÃ©e Fdr, Rosa A L. The DUX4 gene at the FSHD1A locus encodes a pro-apoptotic protein. Neuromuscular disorders: NMD 2007; 17(8):611-623.
Wuebbles R D, Long S W, Hanel M L, Jones P L. Testing the effects of FSHD candidate gene expression in vertebrate muscle development. Int J Clin Exp Pathol 2010; 3(4):386-400.
Shadle S C, Zhong J W, Campbell A E, Conerly M L, Jagannathan S, Wong C J, Morello T D, van der Maarel S M, Tapscott S J. DUX4-induced dsRNA and MYC mRNA stabilization activate apoptotic pathways in human cell models of facioscapulohumeral dystrophy. PLoS Genet 2017; 13(3):e1006658.
Winokur S T, Barrett K, Martin J H, Forrester J R, Simon M, Tawil R, Chung S-A, Masny P S, Figlewicz D A. Facioscapulohumeral muscular dystrophy (FSHD) myoblasts demonstrate increased susceptibility to oxidative stress. Neuromuscular disorders: NMD 2003; 13(4):322-333.
Bou Saada Y, Dib C, Dmitriev P, Hamade A, Carnac G, Laoudj-Chenivesse D, Lipinski M, Vassetzky Y S. Facioscapulohumeral dystrophy myoblasts efficiently repair moderate levels of oxidative DNA damage. Histochem Cell Biol 2016; 145(4):475-483.
Dmitriev P, Bou Saada Y, Dib C, Ansseau E, Barat A, Hamade A, Dessen P, Robert T, Lazar V, Louzada RAN, Dupuy C, Zakharova V, Carnac G, Lipinski M, Vassetzky Y S. DUX4-induced constitutive DNA damage and oxidative stress contribute to aberrant differentiation of myoblasts from FSHD patients. Free Radic Biol Med 2016; 99:244-258.
Kelliher M A, Grimm S, Ishida Y, Kuo F, Stanger B Z, Leder P. The death domain kinase RIP mediates the TNF-induced N F-kappaB signal. Immunity 1998; 8(3):297-303.
Weintraub H, Davis R, Tapscott S, Thayer M, Krause M, Benezra R, Blackwell T K, Turner D, Rupp R, Hollenberg, S. The myoD gene family: nodal point during specification of the muscle cell lineage. Science 1991 251: 761-766.
Cserjesi P, Olson E N. Myogenin induces the myocyte-specific enhancer binding factor MEF-2 independently of other muscle-specific gene products. Mol Cell Biol. 1991 October; 11(10):4854-4862.
Gunning P, Leavitt J, Muscat G, Ng S Y, Kedes L. A human beta-actin expression vector system directs high-level accumulation of antisense transcripts. Proc Natl Acad Sci USA. 1987 July; 84(14):4831-4835.
Johnson J E, Wold B J, Hauschka S D Muscle creatine kinase sequence elements regulating skeletal and cardiac muscle expression in transgenic mice. Mol Cell Biol, 1989, 9: 3393-3399.
Semenza G L, Nejfelt M K, Chi S M, Antonarakis S E. Hypoxia-inducible nuclear factors bind to an enhancer element located 3′ to the human erythropoietin gene. Proc Natl Acad Sci USA. 1991; 88(13):5680-5684.
A steroid-inducible promoter for the controlled overexpression of cloned genes in eukaryotic cells.
Liu Y.-L., Mingozzi F., Rodriguez-Colon S. M., Joseph S., Dobrzynski E., Suzuki T., High K. A., Herzog R. W. Therapeutic levels of factor I X expression using a muscle-specific promoter and adeno-associated virus serotype 1 vector. Hum. Gene Ther. 2004; 15:783-792.
Hauser M. A., Robinson A., Hartigan-O'Connor D., Williams-Gregory D. A., Buskin J. N., Apone S., Kirk C. J., Hardy S., Hauschka S. D., Chamberlain J. S. Analysis of muscle creatine kinase regulatory elements in recombinant adenoviral vectors. Mol. Ther. 2000; 2:16-25.
Himeda C. L., Chen X., Hauschka S. D. Design and testing of regulatory cassettes for optimal activity in skeletal and cardiac muscles. Methods Mol. Biol. 2011; 709:3-19.
Wang B., Li J., Fu F. H., Chen C., Zhu X., Zhou L., Jiang X., Xiao X. Construction and analysis of compact muscle-specific promoters for AAV vectors. Gene Ther. 2008; 15:1489-1499.
McCarty D M. Self-complementary AAV vectors; advances and applications. Mol Ther. 2008; 16(10):1648-1656.
McCarty D M, Monahan P E, Samulski R J, Self-complementary recombinant adeno-associated virus (scAAV) vectors promote efficient transduction independently of DNA synthesis. Gene Ther. 2001; 8: 1248-1254.
Konermann S, Brigham M D, Trevino A E, et al. Genome-scale transcriptional activation by an engineered CRISPR-Cas9 complex. Nature. 2015; 517(7536):583-588.
Kuseu, C., Parlak, M., Tufan, T. et al. CRISPR-STOP: gene silencing through base-editing-induced nonsense mutations. 2017, Nat Methods 14, 710-712.
Joung, J., Sander, J. TALENs: a widely applicable technology for targeted genome editing. 2013, Nat Rev Mol Cell Biol 14, 49-55.
Merienne N, Vachey G, de Longprez L, et al. The Self-Inactivating KamiCas9 System for the Editing of CNS Disease Genes. 2017 Cell Rep. 20(12):2980-2991.
Domitrovich, A, Kunkel, G. Multiple, dispersed human U6 small nuclear RNA genes with varied transcriptional efficiencies. 2003 Nucleic acids research. 31. 2344-52. 10.1093/nar/gkg331.
Malerba, A., Klein, P., Bachtarzi, H. et al. PABPN1 gene therapy for oculopharyngeal muscular dystrophy. 2017 Nat Commun 8, 14848.
Yuan et al Unsaturated Heterocyclic Inhibitors of Necroptosis WO 2010/075290

1. An inhibitor of the necroptosis pathway for use in a method of treating facioscapulohumeral dystrophy (FSHD).

2. The inhibitor for use according to claim 1, wherein the inhibitor reduces activation of the necroptosis pathway.

3. The inhibitor for use according to claim 1 or 2, wherein the inhibitor inhibits gene expression or protein activity of a component of the necroptosis pathway.

4. The inhibitor for use according to any one of the preceding claims wherein the inhibitor inhibits the gene expression of at least one of RIPK3, RIPK1 or MLKL, or wherein the inhibitor inhibits the protein activity of at least one of RIPK3, RIPK1 or MLKL; optionally
wherein the gene is a human gene or the protein is a human protein.

5. The inhibitor for use according to any one of the preceding claims wherein administration of the inhibitor results in an at least 80% reduction of target gene expression or target protein activity in a target cell.

6. The inhibitor for use according to any one of the preceding claims wherein the inhibitor is administered to a target cell of muscular lineage, such as a myoblast, a myotube, or a mature myofibre.

7. The inhibitor for use according to any one of the preceding claims wherein administration of the inhibitor improves or alleviates one or more symptoms of FSHD, including muscle atrophy, muscle weakness, for example abdominal muscle weakness, hip weakness, lower leg weakness including peroneal muscle weakness, shoulder weakness including scapular winging, and/or facial weakness, lordosis, scoliosis, dysphagia, foot drop, inflammation of the muscles, retinal vasculopathy, hearing loss, respiratory involvement or any combination thereof.

8. The inhibitor for use according to any one of the preceding claims, wherein the inhibitor is an antisense oligonucleotide or a small molecule inhibitor.

9. The inhibitor for use according to claim 8 wherein the small molecule inhibitor is selected from:
6E11 (2-[4-(benzyloxy)phenyl]-2,5-dihydroxy-7-methoxy-3,4-dihydro-2H-1-benzopyran-4-one);
compound 1(1-[4-[methyl-[2-(3-sulfamoylanilino)pyrimidin-4-yl]amino]phenyl]-3-[4-(trifluoromethoxy)phenyl]urea);
compound 21 (1-[3-[5-(3-aminophenyl)-1H-pyrrolo[2,3-b]pyridin-3-yl]phenyl]-3-[2-fluoro-5-(trifluoromethyl)phenyl]urea);
compound 22 (7 oxo 2,4,5,7 tetrahydro 6H-pyrazolo[3,4 c]pyridine);
compound 27 (1-[4-(4-aminofuro[2,3-d]pyrimidin-5-yl)phenyl]-3-[2-fluoro-5-(trifluoromethyl)phenyl]urea);
dabrafenib (N-{3-[5-(2-aminopyrimidin-4-yl)-2-tert-butylthiazol-4-yl]-2-fluorophenyl}-2,6-difluorobenzenesulfonamide);
DNL747 (SAR 443060);
fostamatinib ([6-[[5-fluoro-2-(3,4,5-trimethoxyanilino)pyrimidin-4-yl]amino]-2,2-dimethyl-3-oxopyrido[3,2-b][1,4]oxazin-4-yl]methyl dihydrogen phosphate);
galavit (3-aminophthathydrazide monosodium salt);
GSK2982772 (5-benzyl-N-[(3S)-5-methyl-4-oxo-2,3-dihydro-1,5-benzoxazepin-3-yl]-1H-1,2,4-triazole-3-carboxamide);
GSK3145095 ((S)-5-benzyl-N-(7,9-difluoro-2-oxo-2,3,4,5-tetrahydro-1H-benzo[b]azepin-3-yl)-1H-1,2,4-triazole-3-carboxamide);
GSK840 ([4-(5-Methylcarbamoyl-benzoimidazol-1-yl)-phenyl]-acetic acid tert-butyl ester);
GSK843 (3-Benzothiazol-5-yl-7-(2,5-dimethyl-2H-pyrazol-3-yl)-thieno[3,2-c]pyridin-4-ylamine);
GSK872 (Benzothiazol-5-yl-[6-(propane-2-sulfonyl)-quinolin-4-yl]-amine);
GSK963 (2,2-dimethyl-1-[(3S)-3-phenyl-3,4-dihydropyrazol-2-yl]propan-1-one);
GW440139B (4-methyl-3-[(7-pyridin-2-ylquinolin-4-yl)amino]phenol),
GW806742X (3-(4-(Methyl(4-(3-(4-(trifluoromethoxy)phenyl)ureido)phenyl) amino pyrimidin-2-ylamino)benzenesulfonamide);
HS-1371(Quinoline, 4-(4-methylphenoxy)-7-[1-(4-piperidinyl)-1H-pyrazol-4-yl]-)
necrostatin-1 (Nec-1) (5-(1H-indol-3-ylmethyl)-3-methyl-2-sulfanylideneimidazolidin-4-one);
necrostatin-1s (7-Cl—O-Nec-1) (5-[(7-chloro-1H-indol-3-yl)methyl]-3-methylimidazolidine-2,4-dione);
necrostatin-3 (3R,3aR)-rel-2-acetyl-3,3a,4,5-tetrahydro-3-(4-methoxyphenyl)-8-methoxy-2H-benz[g]indazole);
necrostatin-3 analog (1-([3S,3aS]-3-[3-fluoro-4-[trifluoromethoxy]phenyl]-8-methoxy-3,3a,4,5-tetrahydro-2H-benzo[g]indazol-2-yl)-2-hydroxyethanone);
necrostatin-4 ((S)—N-(1-[2-chloro-6-fluorophenyl]ethyl)-5-cyano-1-methyl-1H-pyrrole-2-carboxamide);
necrostatin-5 (5-(1H-indol-3-ylmethyl)-2-thioxo-4-imidazolidinone);
necrosulfonamide (NSA) ((2E)-N-{4-[(3-methoxypyrazin-2-yl)sulfamoyl]phenyl}-3-(5-nitrothiophen-2-yl)prop-2-enamide);
pazopanib (5-[[4-[(2,3-Dimethyl-2H-indazol-6-yl)methylamino]-2-pyrimidinyl]amino]-2-methylbenzolsulfonamide);
PN10 (5-[(7-chloro-1H-indol-3-yl)methyl]-3-[4-[3-(2-imidazo[1,2-b]pyridazin-3-ylethynyl)-4-methylphenyl]butyl]imidazolidine-2,4-dione;
ponatinib (3-(2-imidazo[1,2-b]pyridazin-3-ylethynyl)-4-methyl-N-[4-[(4-methylpiperazin-1-yl)methyl]-3-(trifluoromethyl)phenyl]benzamide);
RIPA-56 (N-benzyl-N-hydroxy-2,2-dimethylbutanamide);
RIPK1 inhibitor 22b (1-(5-{4-Amino-7-ethyl-7H-pyrrolo[2,3-d]pyrimidin-5-yl}-2,3-dihydro-1H-indol-1-yl)-2-[3-(trifluoromethoxy)phenyl] ethan-1-one);
RIPK3 inhibitor 18 (N-{4-[(2-cyclopropaneamidopyridin-4-yl)oxy]-2,3-dimethylphenyl}-1-(4-fluorophenyl)-2-oxo-1,2-dihydropyridine-3-carboxamide);
RIPK3 inhibitor 42 (N-(6-(3-(3-(3-Bromophenyl)ureido)-4-fluorophenoxy)benzo[d]thiazol-2-yl)cyclopropanecarboxamide);
SZM594 (N-[6-(4-fluoro-3-{2-[3-(trifluoromethyl)phenyl]acetamido}phenoxy)-1,3-benzothiazol-2-yl]cyclopropanecarboxamide);
TAK-632 (N-[7-cyano-6-[4-fluoro-3-[[2-[3-(trifluoromethyl)phenyl]acetyl]amino]phenoxy]-1,3-benzothiazol-2-yl]cyclopropanecarboxamide);
tozasertib (VX-680, MK-0457 or N-[4-[4-(4-Methylpiperazin-1-yl)-6-[(5-methyl-1H-pyrazol-3-yl)amino]pyrimidin-2-yl]sulfanylphenyl]cyclopropanecarboxamide);
or any combination thereof;
optionally wherein the inhibitor is selected from one or more of dabrafenib (N-{3-[5-(2-aminopyrimidin-4-yl)-2-tert-butylthiazol-4-yl]-2-fluorophenyl}-2,6-difluorobenzenesulfonamide), necrostatin-1(5-(1H-indol-3-ylmethyl)-3-methyl-2-sulfanylideneimidazolidin-4-one), GSK872 (Benzothiazol-5-yl-[6-(propane-2-sulfonyl)-quinolin-4-yl]-amine) and ponatinib (3-(2-imidazo[1,2-b]pyridazin-3-ylethynyl)-4-methyl-N-[4-[(4-methylpiperazin-1-yl)methyl]-3-(trifluoromethyl)phenyl]benzamide).

10. The antisense oligonucleotide for use according to claim 8 wherein the antisense oligonucleotide targets an RNA molecule encoded by RIPK3, RIPK1 or MLKL.

11. The antisense oligonucleotide for use according to claim 8 or 10, wherein the antisense oligonucleotide targets an RNA molecule comprising or consisting of any one of SEQ ID NO: 1 to 17.

12. The antisense oligonucleotide for use according to any one of claim 8, 10 or 11, wherein the antisense oligonucleotide comprises a sequence that is 100% complementary to a sequence of at least 10, at least 11, at least 12, at least 13, at least 14, at least 15, at least 16, at least 17 or at least 18 contiguous nucleotides of any one of SEQ ID NO: 18 to 35; optionally
wherein the antisense oligonucleotide comprises a sequence that is 100% complementary to the sequence of any one of SEQ ID NOs: 18 to 35.

13. The antisense oligonucleotide for use according to any one of claims 8 or 10 to 12 wherein the antisense oligonucleotide is an shRNA.

14. An expression construct or vector encoding the antisense oligonucleotide of any one of claims 8 or 10 to 13 for use in a method of treating facioscapulohumeral dystrophy (FSHD).

15. The expression construct or vector for use according to claim 14 comprising a nucleotide sequence encoding the antisense oligonucleotide of any one of claim 8 or 10-13 operably linked to a promoter.

16. The expression construct or vector for use according to claim 15, wherein the promoter is selected from an RNA Pol III promoter or a muscle-preferred or muscle-specific promoter, optionally wherein the RNA Pol III promoter is selected from a U6, H1, 7SK promoter, or any variant thereof.

17. The vector for use according to any one of claims 14-16, wherein the vector is a viral vector, optionally wherein the viral vector is an AAV vector, further optionally wherein the serotype of the AAV vector is AAV 1, 6, 8, 9 or rhesus serotype 74 (rh74) or wherein the AAV vector is capsid-free.

18. A vector for use in a method of treating facioscapulohumeral dystrophy (FSHD), wherein the vector comprises a sequence encoding an antisense oligonucleotide comprising a sequence that is 100% complementary to an RNA molecule comprising the sequence of any one of SEQ ID NOs: 18 to 35, said coding sequence being operably linked to an RNA Pol III promoter such that expression of said antisense oligonucleotide in a cell of muscular lineage reduces the expression of at least one of human RIPK3, RIPK1 or MLKL, thereby alleviating at least one symptom of FSHD.

19. A pharmaceutical composition for use in a method of treating FSHD wherein the pharmaceutical composition comprises the small molecule inhibitor of claim 8 or 9, the antisense oligonucleotide of any one of claim 8 or 10-13, the expression construct of any one of claims 14-16, or the vector of any one of claims 14-18, further comprising a pharmaceutically acceptable carrier, diluent, excipient, adjuvant, buffer and/or stabilizer.

20. A vector or pharmaceutical composition comprising a nucleotide sequence encoding an antisense oligonucleotide for targeting at least one of RIPK3, RIPK1 or MLKL, said sequence being operably linked to a promoter.

21. The vector or pharmaceutical composition of claim 20, wherein expression of the antisense oligonucleotide silences the gene expression, transcription, translation and/or protein activity of RIPK3, RIPK1 or MLKL.

22. The vector or pharmaceutical composition of claim 20 or 21, wherein the antisense oligonucleotide targets an RNA molecule comprising or consisting of any one of SEQ ID NO: 1 to 17.

23. The vector or pharmaceutical composition of any one of claims 20-22, wherein the antisense oligonucleotide comprises a sequence that is 100% complementary to a sequence of at least 10, at least 11, at least 12, at least 13, at least 14, at least 15, at least 16, at least 17 or at least 18 contiguous nucleotides selected from any one of SEQ ID NO: 18 to 35.

24. The vector or pharmaceutical composition of any one of claims 20-23, wherein the antisense oligonucleotide comprises a sequence that is 100% complementary to the sequence of any one of SEQ ID NOs: 18 to 35.

25. The vector of any one of claims 20-24, wherein the vector is a viral vector, optionally wherein the viral vector is an AAV vector, and further optionally wherein the AAV vector is capsid-free or has a serotype selected from AAV 1, 6, 8, 9 or rhesus serotype 74 (rh74).


SEQUENCE LISTING (61 sequences)

SEQ ID NO: 1 - RNA, 1872 nt, Homo sapiens
ggaaaaaggguaacaacccggaaaguagacucaccgucuuggucuagagacugaccccugcacagacagaccccuuccccucucugcgaaaggaccaagccccagaagucacuccaucuccuacggcucgcaauuuccagaggcccccuggcaccuuccagccugaugucgugcgucaaguuauggcccagcggugcccccgcccccuugguguccaucgaggaacuggagaaccaggagcucgucggcaaaggcggguucggcacaguguuccgggcgcaacauaggaaguggggcuacgauguggcggucaagaucguaaacucgaaggcgauauccagggaggucaaggccauggcaagucuggauaacgaauucgugcugcgccuagaagggguuaucgagaaggugaacugggaccaagaucccaagccggcucuggugacuaaauucauggagaacggcuccuugucggggcugcugcagucccagugcccucggcccuggccgcuccuuugccgccugcugaaagaaguggugcuugggauguuuuaccugcacgaccagaacccggugcuccugcaccgggaccucaagccauccaacguccugcuggacccagagcugcacgucaagcuggcagauuuuggccuguccacauuucagggaggcucacagucagggacaggguccggggagccagggggcacccugggcuacuuggccccagaacuguuuguuaacguaaaccggaaggccuccacagccagugacgucuacagcuucgggauccuaaugugggcagugcuugcuggaagagaaguugaguugccaaccgaaccaucacucguguacgaagcagugugcaacaggcagaaccggccuucauuggcugagcugccccaagccgggccugagacucccggcuuagaaggacugaaggagcuaaugcagcucugcuggagcagugagcccaaggacagacccuccuuccaggaaugccuaccaaaaacugaugaagucuuccagaugguggagaacaauaugaaugcugcugucuccacgguaaaggauuuccugucucagcucaggagcagcaauaggagauuuucuaucccagagucaggccaaggagggacagaaauggauggcuuuaggagaaccauagaaaaccagcacucucguaaugaugucaugguuucugaguggcuaaacaaacugaaucuagaggagccucccagcucuguuccuaaaaaaugcccgagccuuaccaagaggagcagggcacaagaggagcagguuccacaagccuggacagcaggcacaucuucagauucgauggcccaaccuccccagacuccagagaccucaacuuucagaaaccagaugcccagcccuaccucaacuggaacaccaaguccuggaccccgagggaaucagggggcugagagacaaggcaugaacugguccugcaggaccccggagccaaauccaguaacagggcgaccgcucguuaacauauacaacugcucuggggugcaaguuggagacaacaacuacuugacuaugcaacagacaacugccuugcccacauggggcuuggcaccuucgggcaaggggaggggcuugcagcaccccccaccaguagguucgcaagaaggcccuaaagauccugaagccuggagcaggccacaggguugguauaaucauagcgggaaauaaagcaccuuccaagcuugccuccaagaguuacgaguuaaggaagagugccaccccuugaggccccugacuuccuucuagggcagucuggccugcccacaaacugacuuugugaccugucccccaggagucaauaaacaugauggaaugcua

SEQ ID NO: 2 - RNA, 4017 nt, Homo sapiens
gggaguccgcggcgagcgcagcagcagggcccgguccugcgccucgggagucggcguccaggcucggagcgcgacacggagacuagguggcaggguacagcucugccggggggggaaaaagugguaccauuuugggcguucuugagcuucagaaugcaaccagacauguccuugaaugucauuaagaugaaauccagugacuuccuggagagugcagaacuggacagcggaggcuuugggaaggugucucuguguuuccacagaacccagggacucaugaucaugaaaacaguguacaaggggcccaacugcauugagcacaacgaggcccucuuggaggaggcgaagaugaugaacagacugagacacagccggguggugaagcuccugggcgucaucauagaggaagggaaguacucccuggugauggaguacauggagaagggcaaccugaugcacgugcugaaagccgagaugaguacuccgcuuucuguaaaaggaaggauaauuuuggaaaucauugaaggaaugugcuacuuacauggaaaaggcgugauacacaaggaccugaagccugaaaauauccuuguugauaaugacuuccacauuaagaucgcagaccucggccuugccuccuuuaagauguggagcaaacugaauaaugaagagcacaaugagcugagggaaguggacggcaccgcuaagaagaauggcggcacccucuacuacauggcgcccgagcaccugaaugacgucaacgcaaagcccacagagaagucggauguguacagcuuugcuguaguacucugggcgauauuugcaaauaaggagccauaugaaaaugcuaucugugagcagcaguugauaaugugcauaaaaucugggaacaggccagauguggaugacaucacugaguacugcccaagagaaauuaucagucucaugaagcucugcugggaagcgaauccggaagcucggccgacauuuccuggcauugaagaaaaauuuaggccuuuuuauuuaagucaauuagaagaaaguguagaagaggacgugaagaguuuaaagaaagaguauucaaacgaaaaugcaguugugaagagaaugcagucucuucaacuugauuguguggcaguaccuucaagccggucaaauucagccacagaacagccugguucacugcacaguucccagggacuugggauggguccuguggaggaguccugguuugcuccuucccuggagcacccacaagaagagaaugagcccagccugcagaguaaacuccaagacgaagccaacuaccaucuuuauggcagccgcauggacaggcagacgaaacagcagcccagacagaauguggcuuacaacagagaggaggaaaggagacgcagggucucccaugacccuuuugcacagcaaagaccuuacgagaauuuucagaauacagagggaaaaggcacugcuuauuccagugcagccagucaugguaaugcagugcaccagcccucagggcucaccagccaaccucaaguacuguaucagaacaauggauuauauagcucacauggcuuuggaacaagaccacuggauccaggaacagcaggucccagaguuugguacaggccaauuccaagucauaugccuagucugcauaauaucccagugccugagaccaacuaucuaggaaauacacccaccaugccauucagcuccuugccaccaacagaugaaucuauaaaauauaccauauacaauaguacuggcauucagauuggagccuacaauuauauggagauuggugggacgaguucaucacuacuagacagcacaaauacgaacuucaaagaagagccagcugcuaaguaccaagcuaucuuugauaauaccacuagucugacggauaaacaccuggacccaaucagggaaaaucugggaaagcacuggaaaaacugugcccguaaacugggcuucacacagucucagauugaugaaauugaccaugacuaugagcgagauggacugaaagaaaagguuuaccagaugcuccaaaagugggugaugagggaaggcauaaagggagccacgguggggaagcuggcccaggcgcuccaccaguguuccaggaucgaccuucugagcagcuugauuuacgucagccagaacuaacccuggaugggcuacggcagcugaaguggacgccucacuuaguggauaaccccagaaaguuggcugccucagagcauucagaauucuguccucacugauagggguucugugucugcagaaauuuuguuuccuguacuucauagcuggagaauggggaaagaaaucugcagcaaaggggucucacucuguugccaggcuggucucaaacuucuggacucaagugauccucccgccucggccuuccaaagugcugggauaucaggcacugagccacugcgcccagccaacaauccgcucugaggaaagcguaagcaggaagaccucuuaauggcauagcaccaauaaaaaaaugacuccuaguuguguuuggaaagggagagaagagaugucugaggaaggucauguucuuucagcuuauggcauuuccuagaguuuuguugaagcaagaagaaaaacucagagaauauaaaaucaacuuuuaaaauugugugcucucuucuucacguaggcuccuguuaaaaacaaagugcagucagauucuaagcccuguucagagacuucguggaucacagcugcagcucaccgccacaucacaggauccguuaacguuaauacccaauacucugucagccacuguaggcucuaagaaccacgugcagucuucagcccauuaaauuaucgauuauuuuuuaaugaauugaauuuauauugagucuucaaauuaacugaauggauuuaaagggguaccaaggaggggggaaacaucagaauuucccaggcaguuguugcaaggaauugguacuaaccgugacuacaacaaaaauucuugauugacuuuuaaaguuauuuccuggcauucugguaccuucacccagccugagugcccuggagagggaacaggaaaugcugaucucuaccccugggugagaccagaaccucagggcugauacuguugaguggcuuccucgguuuacucuguguacugugaaaguauuuucauauuuuuucugugugccagagugaaaaaggacagcuucugagugugguaauugugccucuagcacccagccuuucaaagcccaccugaaaccuggggguggaugaaagaacuagaauagaagacugaagcuggguaggccgcucaguguccacuggcauuuugcuaaaccgacaaggaaggcugugugcuuagcucuccccagagggagggcgagaaggguguggugauggucaaucuggcugucggaacagauucuggugucuugggcugauaacaguguuguugauucugauugugaauccccucaacucuagcagacacauacacaccccugaaauggggcugcagagcaggcugucucagccuugccacugucggcaucucggccuggguaauucuguuguggggacuguccuguuccuuguaggauguuuaguagcaucccugcccccaccuacuagaugccaggggcacuguucuccccagccccccgccccaguugugacaauagucucuaaacauugucaaaugguccaaggaaaggggaaaauugccccgguugagaagagcacugcuguaaaguaaugagccucggcucuccugucugcaccuguccgguuacuacuuggccaccacgcagccuuggcuccuacagcccaaaagggagaauggagggaggcuccaggcuuugcuggaggggccugggugaguucuguuugcuccuuguaccaccauccaaaugguguuaucaaaucucuuagauuccaaagagguugaauaauuaauguucaaaggcaagagggcaaggcauuuuuuaacacuuuuuaaaauaaaaauuuauaccacaa

SEQ ID NO: 3 - RNA, 4295 nt, Homo sapiens
gggaguccgcggcgagcgcagcagcagggcccgguccugcgccucgggagucggcguccaggcucggagcgcgacacggagacuagguggcaggaaagaaggcccauaggugcugcuguaugagcuuccuccgcuagacagagcuggccccagccuuuagaugagguguagaaggcugguuacccgggucaccuguagccggccacugugcaacaccaugggcagccuaggaguuuccagaguccccucuccccaaggguacagcucugccggggggggaaaaagugguaccauuuugggcguucuugagcuucagaaugcaaccagacauguccuugaaugucauuaagaugaaauccagugacuuccuggagagugcagaacuggacagcggaggcuuugggaaggugucucuguguuuccacagaacccagggacucaugaucaugaaaacaguguacaaggggcccaacugcauugagcacaacgaggcccucuuggaggaggcgaagaugaugaacagacugagacacagccggguggugaagcuccugggcgucaucauagaggaagggaaguacucccuggugauggaguacauggagaagggcaaccugaugcacgugcugaaagccgagaugaguacuccgcuuucuguaaaaggaaggauaauuuuggaaaucauugaaggaaugugcuacuuacauggaaaaggcgugauacacaaggaccugaagccugaaaauauccuuguugauaaugacuuccacauuaagguucaggcuacagcucaaaacaagcaacacugcaguccuuauccucagugugucuuugggauccuuggacacuugcauccaugggccucuggacgccauuuggggaaauagaagaucgcagaccucggccuugccuccuuuaagauguggagcaaacugaauaaugaagagcacaaugagcugagggaaguggacggcaccgcuaagaagaauggcggcacccucuacuacauggcgcccgagcaccugaaugacgucaacgcaaagcccacagagaagucggauguguacagcuuugcuguaguacucugggcgauauuugcaaauaaggagccauaugaaaaugcuaucugugagcagcaguugauaaugugcauaaaaucugggaacaggccagauguggaugacaucacugaguacugcccaagagaaauuaucagucucaugaagcucugcugggaagcgaauccggaagcucggccgacauuuccuggcauugaagaaaaauuuaggccuuuuuauuuaagucaauuagaagaaaguguagaagaggacgugaagaguuuaaagaaagaguauucaaacgaaaaugcaguugugaagagaaugcagucucuucaacuugauuguguggcaguaccuucaagccggucaaauucagccacagaacagccugguucacugcacaguucccagggacuugggauggguccuguggaggaguccugguuugcuccuucccuggagcacccacaagaagagaaugagcccagccugcagaguaaacuccaagacgaagccaacuaccaucuuuauggcagccgcauggacaggcagacgaaacagcagcccagacagaauguggcuuacaacagagaggaggaaaggagacgcagggucucccaugacccuuuugcacagcaaagaccuuacgagaauuuucagaauacagagggaaaaggcacugcuuauuccagugcagccagucaugguaaugcagugcaccagcccucagggcucaccagccaaccucaaguacuguaucagaacaauggauuauauagcucacauggcuuuggaacaagaccacuggauccaggaacagcaggucccagaguuugguacaggccaauuccaagucauaugccuagucugcauaauaucccagugccugagaccaacuaucuaggaaauacacccaccaugccauucagcuccuugccaccaacagaugaaucuauaaaauauaccauauacaauaguacuggcauucagauuggagccuacaauuauauggagauuggugggacgaguucaucacuacuagacagcacaaauacgaacuucaaagaagagccagcugcuaaguaccaagcuaucuuugauaauaccacuagucugacggauaaacaccuggacccaaucagggaaaaucugggaaagcacuggaaaaacugugcccguaaacugggcuucacacagucucagauugaugaaauugaccaugacuaugagcgagauggacugaaagaaaagguuuaccagaugcuccaaaagugggugaugagggaaggcauaaagggagccacgguggggaagcuggcccaggcgcuccaccaguguuccaggaucgaccuucugagcagcuugauuuacgucagccagaacuaacccuggaugggcuacggcagcugaaguggacgccucacuuaguggauaaccccagaaaguuggcugccucagagcauucagaauucuguccucacugauagggguucugugucugcagaaauuuuguuuccuguacuucauagcuggagaauggggaaagaaaucugcagcaaaggggucucacucuguugccaggcuggucucaaacuucuggacucaagugauccucccgccucggccuuccaaagugcugggauaucaggcacugagccacugcgcccagccaacaauccgcucugaggaaagcguaagcaggaagaccucuuaauggcauagcaccaauaaaaaaaugacuccuaguuguguuuggaaagggagagaagagaugucugaggaaggucauguucuuucagcuuauggcauuuccuagaguuuuguugaagcaagaagaaaaacucagagaauauaaaaucaacuuuuaaaauugugugcucucuucuucacguaggcuccuguuaaaaacaaagugcagucagauucuaagcccuguucagagacuucguggaucacagcugcagcucaccgccacaucacaggauccguuaacguuaauacccaauacucugucagccacuguaggcucuaagaaccacgugcagucuucagcccauuaaauuaucgauuauuuuuuaaugaauugaauuuauauugagucuucaaauuaacugaauggauuuaaagggguaccaaggaggggggaaacaucagaauuucccaggcaguuguugcaaggaauugguacuaaccgugacuacaacaaaaauucuugauugacuuuuaaaguuauuuccuggcauucugguaccuucacccagccugagugcccuggagagggaacaggaaaugcugaucucuaccccugggugagaccagaaccucagggcugauacuguugaguggcuuccucgguuuacucuguguacugugaaaguauuuucauauuuuuucugugugccagagugaaaaaggacagcuucugagugugguaauugugccucuagcacccagccuuucaaagcccaccugaaaccuggggguggaugaaagaacuagaauagaagacugaagcuggguaggccgcucaguguccacuggcauuuugcuaaaccgacaaggaaggcugugugcuuagcucuccccagagggagggcgagaaggguguggugauggucaaucuggcugucggaacagauucuggugucuugggcugauaacaguguuguugauucugauugugaauccccucaacucuagcagacacauacacaccccugaaauggggcugcagagcaggcugucucagccuugccacugucggcaucucggccuggguaauucuguuguggggacuguccuguuccuuguaggauguuuaguagcaucccugcccccaccuacuagaugccaggggcacuguucuccccagccccccgccccaguugugacaauagucucuaaacauugucaaaugguccaaggaaaggggaaaauugccccgguugagaagagcacugcuguaaaguaaugagccucggcucuccugucugcaccuguccgguuacuacuuggccaccacgcagccuuggcuccuacagcccaaaagggagaauggagggaggcuccaggcuuugcuggaggggccugggugaguucuguuugcuccuuguaccaccauccaaaugguguuaucaaaucucuuagauuccaaagagguugaauaauuaauguucaaaggcaagagggcaaggcauuuuuuaacacuuuuuaaaauaaaaauuuauaccacaa

SEQ ID NO: 4 - RNA, 4092 nt, Homo sapiens
aguccacgcccuccagccgggcgcgcucgacgcggacggcgggccagcugccggagcgcggcgacuccaggggacccacagcuggggcgccagagcgcggccauccgggcggggccgacggagcgcggcaggacuuggcuggacggcgcggccacggagaagggcgcggguacagcucugccggggggggaaaaagugguaccauuuugggcguucuugagcuucagaaugcaaccagacauguccuugaaugucauuaagaugaaauccagugacuuccuggagagugcagaacuggacagcggaggcuuugggaaggugucucuguguuuccacagaacccagggacucaugaucaugaaaacaguguacaaggggcccaacugcauugagcacaacgaggcccucuuggaggaggcgaagaugaugaacagacugagacacagccggguggugaagcuccugggcgucaucauagaggaagggaaguacucccuggugauggaguacauggagaagggcaaccugaugcacgugcugaaagccgagaugaguacuccgcuuucuguaaaaggaaggauaauuuuggaaaucauugaaggaaugugcuacuuacauggaaaaggcgugauacacaaggaccugaagccugaaaauauccuuguugauaaugacuuccacauuaagaucgcagaccucggccuugccuccuuuaagauguggagcaaacugaauaaugaagagcacaaugagcugagggaaguggacggcaccgcuaagaagaauggcggcacccucuacuacauggcgcccgagcaccugaaugacgucaacgcaaagcccacagagaagucggauguguacagcuuugcuguaguacucugggcgauauuugcaaauaaggagccauaugaaaaugcuaucugugagcagcaguugauaaugugcauaaaaucugggaacaggccagauguggaugacaucacugaguacugcccaagagaaauuaucagucucaugaagcucugcugggaagcgaauccggaagcucggccgacauuuccuggcauugaagaaaaauuuaggccuuuuuauuuaagucaauuagaagaaaguguagaagaggacgugaagaguuuaaagaaagaguauucaaacgaaaaugcaguugugaagagaaugcagucucuucaacuugauuguguggcaguaccuucaagccggucaaauucagccacagaacagccugguucacugcacaguucccagggacuugggauggguccuguggaggaguccugguuugcuccuucccuggagcacccacaagaagagaaugagcccagccugcagaguaaacuccaagacgaagccaacuaccaucuuuauggcagccgcauggacaggcagacgaaacagcagcccagacagaauguggcuuacaacagagaggaggaaaggagacgcagggucucccaugacccuuuugcacagcaaagaccuuacgagaauuuucagaauacagagggaaaaggcacugcuuauuccagugcagccagucaugguaaugcagugcaccagcccucagggcucaccagccaaccucaaguacuguaucagaacaauggauuauauagcucacauggcuuuggaacaagaccacuggauccaggaacagcaggucccagaguuugguacaggccaauuccaagucauaugccuagucugcauaauaucccagugccugagaccaacuaucuaggaaauacacccaccaugccauucagcuccuugccaccaacagaugaaucuauaaaauauaccauauacaauaguacuggcauucagauuggagccuacaauuauauggagauuggugggacgaguucaucacuacuagacagcacaaauacgaacuucaaagaagagccagcugcuaaguaccaagcuaucuuugauaauaccacuagucugacggauaaacaccuggacccaaucagggaaaaucugggaaagcacuggaaaaacugugcccguaaacugggcuucacacagucucagauugaugaaauugaccaugacuaugagcgagauggacugaaagaaaagguuuaccagaugcuccaaaagugggugaugagggaaggcauaaagggagccacgguggggaagcuggcccaggcgcuccaccaguguuccaggaucgaccuucugagcagcuugauuuacgucagccagaacuaacccuggaugggcuacggcagcugaaguggacgccucacuuaguggauaaccccagaaaguuggcugccucagagcauucagaauucuguccucacugauagggguucugugucugcagaaauuuuguuuccuguacuucauagcuggagaauggggaaagaaaucugcagcaaaggggucucacucuguugccaggcuggucucaaacuucuggacucaagugauccucccgccucggccuuccaaagugcugggauaucaggcacugagccacugcgcccagccaacaauccgcucugaggaaagcguaagcaggaagaccucuuaauggcauagcaccaauaaaaaaaugacuccuaguuguguuuggaaagggagagaagagaugucugaggaaggucauguucuuucagcuuauggcauuuccuagaguuuuguugaagcaagaagaaaaacucagagaauauaaaaucaacuuuuaaaauugugugcucucuucuucacguaggcuccuguuaaaaacaaagugcagucagauucuaagcccuguucagagacuucguggaucacagcugcagcucaccgccacaucacaggauccguuaacguuaauacccaauacucugucagccacuguaggcucuaagaaccacgugcagucuucagcccauuaaauuaucgauuauuuuuuaaugaauugaauuuauauugagucuucaaauuaacugaauggauuuaaagggguaccaaggaggggggaaacaucagaauuucccaggcaguuguugcaaggaauugguacuaaccgugacuacaacaaaaauucuugauugacuuuuaaaguuauuuccuggcauucugguaccuucacccagccugagugcccuggagagggaacaggaaaugcugaucucuaccccugggugagaccagaaccucagggcugauacuguugaguggcuuccucgguuuacucuguguacugugaaaguauuuucauauuuuuucugugugccagagugaaaaaggacagcuucugagugugguaauugugccucuagcacccagccuuucaaagcccaccugaaaccuggggguggaugaaagaacuagaauagaagacugaagcuggguaggccgcucaguguccacuggcauuuugcuaaaccgacaaggaaggcugugugcuuagcucuccccagagggagggcgagaaggguguggugauggucaaucuggcugucggaacagauucuggugucuugggcugauaacaguguuguugauucugauugugaauccccucaacucuagcagacacauacacaccccugaaauggggcugcagagcaggcugucucagccuugccacugucggcaucucggccuggguaauucuguuguggggacuguccuguuccuuguaggauguuuaguagcaucccugcccccaccuacuagaugccaggggcacuguucuccccagccccccgccccaguugugacaauagucucuaaacauugucaaaugguccaaggaaaggggaaaauugccccgguugagaagagcacugcuguaaaguaaugagccucggcucuccugucugcaccuguccgguuacuacuuggccaccacgcagccuuggcuccuacagcccaaaagggagaauggagggaggcuccaggcuuugcuggaggggccugggugaguucuguuugcuccuuguaccaccauccaaaugguguuaucaaaucucuuagauuccaaagagguugaauaauuaauguucaaaggcaagagggcaaggcauuuuuuaacacuuuuuaaaauaaaaauuuauaccacaa

SEQ ID NO: 5 - RNA, 3879 nt, Homo sapiens
gggaguccgcggcgagcgcagcagcagggcccgguccugcgccucgggagucggcguccaggcucggagcgcgacacggagacuagguggcaggguacagcucugccggggggggaaaaagugguaccauuuugggcguucuugagcuucagaaugcaaccagacauguccuugaaugucauuaagaugaaauccagugacuuccuggagagugcagaacuggacagcggaggcuuugggaaggugucucuguguuuccacagaacccagggacucaugaucaugaaaacaguguacaaggggcccaacugcauugagcacaacgaggcccucuuggaggaggcgaagaugaugaacagacugagacacagccggguggugaagcuccugggcgucaucauagaggaagggaaguacucccuggugauggaguacauggagaagggcaaccugaugcacgugcugaaagccgagaucgcagaccucggccuugccuccuuuaagauguggagcaaacugaauaaugaagagcacaaugagcugagggaaguggacggcaccgcuaagaagaauggcggcacccucuacuacauggcgcccgagcaccugaaugacgucaacgcaaagcccacagagaagucggauguguacagcuuugcuguaguacucugggcgauauuugcaaauaaggagccauaugaaaaugcuaucugugagcagcaguugauaaugugcauaaaaucugggaacaggccagauguggaugacaucacugaguacugcccaagagaaauuaucagucucaugaagcucugcugggaagcgaauccggaagcucggccgacauuuccuggcauugaagaaaaauuuaggccuuuuuauuuaagucaauuagaagaaaguguagaagaggacgugaagaguuuaaagaaagaguauucaaacgaaaaugcaguugugaagagaaugcagucucuucaacuugauuguguggcaguaccuucaagccggucaaauucagccacagaacagccugguucacugcacaguucccagggacuugggauggguccuguggaggaguccugguuugcuccuucccuggagcacccacaagaagagaaugagcccagccugcagaguaaacuccaagacgaagccaacuaccaucuuuauggcagccgcauggacaggcagacgaaacagcagcccagacagaauguggcuuacaacagagaggaggaaaggagacgcagggucucccaugacccuuuugcacagcaaagaccuuacgagaauuuucagaauacagagggaaaaggcacugcuuauuccagugcagccagucaugguaaugcagugcaccagcccucagggcucaccagccaaccucaaguacuguaucagaacaauggauuauauagcucacauggcuuuggaacaagaccacuggauccaggaacagcaggucccagaguuugguacaggccaauuccaagucauaugccuagucugcauaauaucccagugccugagaccaacuaucuaggaaauacacccaccaugccauucagcuccuugccaccaacagaugaaucuauaaaauauaccauauacaauaguacuggcauucagauuggagccuacaauuauauggagauuggugggacgaguucaucacuacuagacagcacaaauacgaacuucaaagaagagccagcugcuaaguaccaagcuaucuuugauaauaccacuagucugacggauaaacaccuggacccaaucagggaaaaucugggaaagcacuggaaaaacugugcccguaaacugggcuucacacagucucagauugaugaaauugaccaugacuaugagcgagauggacugaaagaaaagguuuaccagaugcuccaaaagugggugaugagggaaggcauaaagggagccacgguggggaagcuggcccaggcgcuccaccaguguuccaggaucgaccuucugagcagcuugauuuacgucagccagaacuaacccuggaugggcuacggcagcugaaguggacgccucacuuaguggauaaccccagaaaguuggcugccucagagcauucagaauucuguccucacugauagggguucugugucugcagaaauuuuguuuccuguacuucauagcuggagaauggggaaagaaaucugcagcaaaggggucucacucuguugccaggcuggucucaaacuucuggacucaagugauccucccgccucggccuuccaaagugcugggauaucaggcacugagccacugcgcccagccaacaauccgcucugaggaaagcguaagcaggaagaccucuuaauggcauagcaccaauaaaaaaaugacuccuaguuguguuuggaaagggagagaagagaugucugaggaaggucauguucuuucagcuuauggcauuuccuagaguuuuguugaagcaagaagaaaaacucagagaauauaaaaucaacuuuuaaaauugugugcucucuucuucacguaggcuccuguuaaaaacaaagugcagucagauucuaagcccuguucagagacuucguggaucacagcugcagcucaccgccacaucacaggauccguuaacguuaauacccaauacucugucagccacuguaggcucuaagaaccacgugcagucuucagcccauuaaauuaucgauuauuuuuuaaugaauugaauuuauauugagucuucaaauuaacugaauggauuuaaagggguaccaaggaggggggaaacaucagaauuucccaggcaguuguugcaaggaauugguacuaaccgugacuacaacaaaaauucuugauugacuuuuaaaguuauuuccuggcauucugguaccuucacccagccugagugcccuggagagggaacaggaaaugcugaucucuaccccugggugagaccagaaccucagggcugauacuguugaguggcuuccucgguuuacucuguguacugugaaaguauuuucauauuuuuucugugugccagagugaaaaaggacagcuucugagugugguaauugugccucuagcacccagccuuucaaagcccaccugaaaccuggggguggaugaaagaacuagaauagaagacugaagcuggguaggccgcucaguguccacuggcauuuugcuaaaccgacaaggaaggcugugugcuuagcucuccccagagggagggcgagaaggguguggugauggucaaucuggcugucggaacagauucuggugucuugggcugauaacaguguuguugauucugauugugaauccccucaacucuagcagacacauacacaccccugaaauggggcugcagagcaggcugucucagccuugccacugucggcaucucggccuggguaauucuguuguggggacuguccuguuccuuguaggauguuuaguagcaucccugcccccaccuacuagaugccaggggcacuguucuccccagccccccgccccaguugugacaauagucucuaaacauugucaaaugguccaaggaaaggggaaaauugccccgguugagaagagcacugcuguaaaguaaugagccucggcucuccugucugcaccuguccgguuacuacuuggccaccacgcagccuuggcuccuacagcccaaaagggagaauggagggaggcuccaggcuuugcuggaggggccugggugaguucuguuugcuccuuguaccaccauccaaaugguguuaucaaaucucuuagauuccaaagagguugaauaauuaauguucaaaggcaagagggcaaggcauuuuuuaacacuuuuuaaaauaaaaauuuauaccacaa

SEQ ID NO: 6 - RNA, 4131 nt, Homo sapiens
gggaguccgcggcgagcgcagcagcagggcccgguccugcgccucgggagucggcguccaggcucggagcgcgacacggagacuagguggcaggguacagcucugccggggggggaaaaagugguaccauuuugggcguucuugagcuucagaaugcaaccagacauguccuugaaugucauuaagaugaaauccagugacuuccuggagagugcagaacuggacagcggaggcuuugggaaggugucucuguguuuccacagaacccagggacucaugaucaugaaaacaguguacaaggggcccaacugcauugagcacaacgaggcccucuuggaggaggcgaagaugaugaacagacugagacacagccggguggugaagcuccugggcgucaucauagaggaagggaaguacucccuggugauggaguacauggagaagggcaaccugaugcacgugcugaaagccgagaugaguacuccgcuuucuguaaaaggaaggauaauuuuggaaaucauugaaggaaugugcuacuuacauggaaaaggcgugauacacaaggaccugaagccugaaaauauccuuguugauaaugacuuccacauuaagguucaggcuacagcucaaaacaagcaacacugcaguccuuauccucagugugucuuugggauccuuggacacuugcauccaugggccucuggacgccauuuggggaaauagaagaucgcagaccucggccuugccuccuuuaagauguggagcaaacugaauaaugaagagcacaaugagcugagggaaguggacggcaccgcuaagaagaauggcggcacccucuacuacauggcgcccgagcaccugaaugacgucaacgcaaagcccacagagaagucggauguguacagcuuugcuguaguacucugggcgauauuugcaaauaaggagccauaugaaaaugcuaucugugagcagcaguugauaaugugcauaaaaucugggaacaggccagauguggaugacaucacugaguacugcccaagagaaauuaucagucucaugaagcucugcugggaagcgaauccggaagcucggccgacauuuccuggcauugaagaaaaauuuaggccuuuuuauuuaagucaauuagaagaaaguguagaagaggacgugaagaguuuaaagaaagaguauucaaacgaaaaugcaguugugaagagaaugcagucucuucaacuugauuguguggcaguaccuucaagccggucaaauucagccacagaacagccugguucacugcacaguucccagggacuugggauggguccuguggaggaguccugguuugcuccuucccuggagcacccacaagaagagaaugagcccagccugcagaguaaacuccaagacgaagccaacuaccaucuuuauggcagccgcauggacaggcagacgaaacagcagcccagacagaauguggcuuacaacagagaggaggaaaggagacgcagggucucccaugacccuuuugcacagcaaagaccuuacgagaauuuucagaauacagagggaaaaggcacugcuuauuccagugcagccagucaugguaaugcagugcaccagcccucagggcucaccagccaaccucaaguacuguaucagaacaauggauuauauagcucacauggcuuuggaacaagaccacuggauccaggaacagcaggucccagaguuugguacaggccaauuccaagucauaugccuagucugcauaauaucccagugccugagaccaacuaucuaggaaauacacccaccaugccauucagcuccuugccaccaacagaugaaucuauaaaauauaccauauacaauaguacuggcauucagauuggagccuacaauuauauggagauuggugggacgaguucaucacuacuagacagcacaaauacgaacuucaaagaagagccagcugcuaaguaccaagcuaucuuugauaauaccacuagucugacggauaaacaccuggacccaaucagggaaaaucugggaaagcacuggaaaaacugugcccguaaacugggcuucacacagucucagauugaugaaauugaccaugacuaugagcgagauggacugaaagaaaagguuuaccagaugcuccaaaagugggugaugagggaaggcauaaagggagccacgguggggaagcuggcccaggcgcuccaccaguguuccaggaucgaccuucugagcagcuugauuuacgucagccagaacuaacccuggaugggcuacggcagcugaaguggacgccucacuuaguggauaaccccagaaaguuggcugccucagagcauucagaauucuguccucacugauagggguucugugucugcagaaauuuuguuuccuguacuucauagcuggagaauggggaaagaaaucugcagcaaaggggucucacucuguugccaggcuggucucaaacuucuggacucaagugauccucccgccucggccuuccaaagugcugggauaucaggcacugagccacugcgcccagccaacaauccgcucugaggaaagcguaagcaggaagaccucuuaauggcauagcaccaauaaaaaaaugacuccuaguuguguuuggaaagggagagaagagaugucugaggaaggucauguucuuucagcuuauggcauuuccuagaguuuuguugaagcaagaagaaaaacucagagaauauaaaaucaacuuuuaaaauugugugcucucuucuucacguaggcuccuguuaaaaacaaagugcagucagauucuaagcccuguucagagacuucguggaucacagcugcagcucaccgccacaucacaggauccguuaacguuaauacccaauacucugucagccacuguaggcucuaagaaccacgugcagucuucagcccauuaaauuaucgauuauuuuuuaaugaauugaauuuauauugagucuucaaauuaacugaauggauuuaaagggguaccaaggaggggggaaacaucagaauuucccaggcaguuguugcaaggaauugguacuaaccgugacuacaacaaaaauucuugauugacuuuuaaaguuauuuccuggcauucugguaccuucacccagccugagugcccuggagagggaacaggaaaugcugaucucuaccccugggugagaccagaaccucagggcugauacuguugaguggcuuccucgguuuacucuguguacugugaaaguauuuucauauuuuuucugugugccagagugaaaaaggacagcuucugagugugguaauugugccucuagcacccagccuuucaaagcccaccugaaaccuggggguggaugaaagaacuagaauagaagacugaagcuggguaggccgcucaguguccacuggcauuuugcuaaaccgacaaggaaggcugugugcuuagcucuccccagagggagggcgagaaggguguggugauggucaaucuggcugucggaacagauucuggugucuugggcugauaacaguguuguugauucugauugugaauccccucaacucuagcagacacauacacaccccugaaauggggcugcagagcaggcugucucagccuugccacugucggcaucucggccuggguaauucuguuguggggacuguccuguuccuuguaggauguuuaguagcaucccugcccccaccuacuagaugccaggggcacuguucuccccagccccccgccccaguugugacaauagucucuaaacauugucaaaugguccaaggaaaggggaaaauugccccgguugagaagagcacugcuguaaaguaaugagccucggcucuccugucugcaccuguccgguuacuacuuggccaccacgcagccuuggcuccuacagcccaaaagggagaauggagggaggcuccaggcuuugcuggaggggccugggugaguucuguuugcuccuuguaccaccauccaaaugguguuaucaaaucucuuagauuccaaagagguugaauaauuaauguucaaaggcaagagggcaaggcauuuuuuaacacuuuuuaaaauaaaaauuuauaccacaa

SEQ ID NO: 7 - RNA, 4206 nt, Homo sapiens
aguccacgcccuccagccgggcgcgcucgacgcggacggcgggccagcugccggagcgcggcgacuccaggggacccacagcuggggcgccagagcgcggccauccgggcggggccgacggagcgcggcaggacuuggcuggacggcgcggccacggagaagggcgcggguacagcucugccggggggggaaaaagugguaccauuuugggcguucuugagcuucagaaugcaaccagacauguccuugaaugucauuaagaugaaauccagugacuuccuggagagugcagaacuggacagcggaggcuuugggaaggugucucuguguuuccacagaacccagggacucaugaucaugaaaacaguguacaaggggcccaacugcauugagcacaacgaggcccucuuggaggaggcgaagaugaugaacagacugagacacagccggguggugaagcuccugggcgucaucauagaggaagggaaguacucccuggugauggaguacauggagaagggcaaccugaugcacgugcugaaagccgagaugaguacuccgcuuucuguaaaaggaaggauaauuuuggaaaucauugaaggaaugugcuacuuacauggaaaaggcgugauacacaaggaccugaagccugaaaauauccuuguugauaaugacuuccacauuaagguucaggcuacagcucaaaacaagcaacacugcaguccuuauccucagugugucuuugggauccuuggacacuugcauccaugggccucuggacgccauuuggggaaauagaagaucgcagaccucggccuugccuccuuuaagauguggagcaaacugaauaaugaagagcacaaugagcugagggaaguggacggcaccgcuaagaagaauggcggcacccucuacuacauggcgcccgagcaccugaaugacgucaacgcaaagcccacagagaagucggauguguacagcuuugcuguaguacucugggcgauauuugcaaauaaggagccauaugaaaaugcuaucugugagcagcaguugauaaugugcauaaaaucugggaacaggccagauguggaugacaucacugaguacugcccaagagaaauuaucagucucaugaagcucugcugggaagcgaauccggaagcucggccgacauuuccuggcauugaagaaaaauuuaggccuuuuuauuuaagucaauuagaagaaaguguagaagaggacgugaagaguuuaaagaaagaguauucaaacgaaaaugcaguugugaagagaaugcagucucuucaacuugauuguguggcaguaccuucaagccggucaaauucagccacagaacagccugguucacugcacaguucccagggacuugggauggguccuguggaggaguccugguuugcuccuucccuggagcacccacaagaagagaaugagcccagccugcagaguaaacuccaagacgaagccaacuaccaucuuuauggcagccgcauggacaggcagacgaaacagcagcccagacagaauguggcuuacaacagagaggaggaaaggagacgcagggucucccaugacccuuuugcacagcaaagaccuuacgagaauuuucagaauacagagggaaaaggcacugcuuauuccagugcagccagucaugguaaugcagugcaccagcccucagggcucaccagccaaccucaaguacuguaucagaacaauggauuauauagcucacauggcuuuggaacaagaccacuggauccaggaacagcaggucccagaguuugguacaggccaauuccaagucauaugccuagucugcauaauaucccagugccugagaccaacuaucuaggaaauacacccaccaugccauucagcuccuugccaccaacagaugaaucuauaaaauauaccauauacaauaguacuggcauucagauuggagccuacaauuauauggagauuggugggacgaguucaucacuacuagacagcacaaauacgaacuucaaagaagagccagcugcuaaguaccaagcuaucuuugauaauaccacuagucugacggauaaacaccuggacccaaucagggaaaaucugggaaagcacuggaaaaacugugcccguaaacugggcuucacacagucucagauugaugaaauugaccaugacuaugagcgagauggacugaaagaaaagguuuaccagaugcuccaaaagugggugaugagggaaggcauaaagggagccacgguggggaagcuggcccaggcgcuccaccaguguuccaggaucgaccuucugagcagcuugauuuacgucagccagaacuaacccuggaugggcuacggcagcugaaguggacgccucacuuaguggauaaccccagaaaguuggcugccucagagcauucagaauucuguccucacugauagggguucugugucugcagaaauuuuguuuccuguacuucauagcuggagaauggggaaagaaaucugcagcaaaggggucucacucuguugccaggcuggucucaaacuucuggacucaagugauccucccgccucggccuuccaaagugcugggauaucaggcacugagccacugcgcccagccaacaauccgcucugaggaaagcguaagcaggaagaccucuuaauggcauagcaccaauaaaaaaaugacuccuaguuguguuuggaaagggagagaagagaugucugaggaaggucauguucuuucagcuuauggcauuuccuagaguuuuguugaagcaagaagaaaaacucagagaauauaaaaucaacuuuuaaaauugugugcucucuucuucacguaggcuccuguuaaaaacaaagugcagucagauucuaagcccuguucagagacuucguggaucacagcugcagcucaccgccacaucacaggauccguuaacguuaauacccaauacucugucagccacuguaggcucuaagaaccacgugcagucuucagcccauuaaauuaucgauuauuuuuuaaugaauugaauuuauauugagucuucaaauuaacugaauggauuuaaagggguaccaaggaggggggaaacaucagaauuucccaggcaguuguugcaaggaauugguacuaaccgugacuacaacaaaaauucuugauugacuuuuaaaguuauuuccuggcauucugguaccuucacccagccugagugcccuggagagggaacaggaaaugcugaucucuaccccugggugagaccagaaccucagggcugauacuguugaguggcuuccucgguuuacucuguguacugugaaaguauuuucauauuuuuucugugugccagagugaaaaaggacagcuucugagugugguaauugugccucuagcacccagccuuucaaagcccaccugaaaccuggggguggaugaaagaacuagaauagaagacugaagcuggguaggccgcucaguguccacuggcauuuugcuaaaccgacaaggaaggcugugugcuuagcucuccccagagggagggcgagaaggguguggugauggucaaucuggcugucggaacagauucuggugucuugggcugauaacaguguuguugauucugauugugaauccccucaacucuagcagacacauacacaccccugaaauggggcugcagagcaggcugucucagccuugccacugucggcaucucggccuggguaauucuguuguggggacuguccuguuccuuguaggauguuuaguagcaucccugcccccaccuacuagaugccaggggcacuguucuccccagccccccgccccaguugugacaauagucucuaaacauugucaaaugguccaaggaaaggggaaaauugccccgguugagaagagcacugcuguaaaguaaugagccucggcucuccugucugcaccuguccgguuacuacuuggccaccacgcagccuuggcuccuacagcccaaaagggagaauggagggaggcuccaggcuuugcuggaggggccugggugaguucuguuugcuccuuguaccaccauccaaaugguguuaucaaaucucuuagauuccaaagagguugaauaauuaauguucaaaggcaagagggcaaggcauuuuuuaacacuuuuuaaaauaaaaauuuauaccacaa

SEQ ID NO: 8 - RNA, 4517 nt, Homo sapiens
gcaagagagcuccgggacucagaccccgggcgcgaggucccacgagcggcggggcggcgcucucgcgguccuccuccccggcaagggccggggcagggggaggaggcagcgcgaacaguccacgcccuccagccgggcgcgcucgacgcggacggcgggccagcugccggagcgcggcgacuccaggggacccacagcuggggcgccagagcgcggccauccgggcggggccgacggagcgcggcaggacuuggcuggacggcgcggccacggagaagggcgcggugagcggacuggcaccgcgcggggaacauuccggaggccgccgggcucagucccggugaccccccuggucgggguucgcgcgcccucccccagcacgcccgggcaaggcugccgcggcgucggaguaauccccaacccuccgccgccgcuucuucccucacccgggccuccagacguuugccgcggaaacgaaagguacagcucugccggggggggaaaaagugguaccauuuugggcguucuugagcuucagaaugcaaccagacauguccuugaaugucauuaagaugaaauccagugacuuccuggagagugcagaacuggacagcggaggcuuugggaaggugucucuguguuuccacagaacccagggacucaugaucaugaaaacaguguacaaggggcccaacugcauugagcacaacgaggcccucuuggaggaggcgaagaugaugaacagacugagacacagccggguggugaagcuccugggcgucaucauagaggaagggaaguacucccuggugauggaguacauggagaagggcaaccugaugcacgugcugaaagccgagaugaguacuccgcuuucuguaaaaggaaggauaauuuuggaaaucauugaaggaaugugcuacuuacauggaaaaggcgugauacacaaggaccugaagccugaaaauauccuuguugauaaugacuuccacauuaagguucaggcuacagcucaaaacaagcaacacugcaguccuuauccucagugugucuuugggauccuuggacacuugcauccaugggccucuggacgccauuuggggaaauagaagaucgcagaccucggccuugccuccuuuaagauguggagcaaacugaauaaugaagagcacaaugagcugagggaaguggacggcaccgcuaagaagaauggcggcacccucuacuacauggcgcccgagcaccugaaugacgucaacgcaaagcccacagagaagucggauguguacagcuuugcuguaguacucugggcgauauuugcaaauaaggagccauaugaaaaugcuaucugugagcagcaguugauaaugugcauaaaaucugggaacaggccagauguggaugacaucacugaguacugcccaagagaaauuaucagucucaugaagcucugcugggaagcgaauccggaagcucggccgacauuuccuggcauugaagaaaaauuuaggccuuuuuauuuaagucaauuagaagaaaguguagaagaggacgugaagaguuuaaagaaagaguauucaaacgaaaaugcaguugugaagagaaugcagucucuucaacuugauuguguggcaguaccuucaagccggucaaauucagccacagaacagccugguucacugcacaguucccagggacuugggauggguccuguggaggaguccugguuugcuccuucccuggagcacccacaagaagagaaugagcccagccugcagaguaaacuccaagacgaagccaacuaccaucuuuauggcagccgcauggacaggcagacgaaacagcagcccagacagaauguggcuuacaacagagaggaggaaaggagacgcagggucucccaugacccuuuugcacagcaaagaccuuacgagaauuuucagaauacagagggaaaaggcacugcuuauuccagugcagccagucaugguaaugcagugcaccagcccucagggcucaccagccaaccucaaguacuguaucagaacaauggauuauauagcucacauggcuuuggaacaagaccacuggauccaggaacagcaggucccagaguuugguacaggccaauuccaagucauaugccuagucugcauaauaucccagugccugagaccaacuaucuaggaaauacacccaccaugccauucagcuccuugccaccaacagaugaaucuauaaaauauaccauauacaauaguacuggcauucagauuggagccuacaauuauauggagauuggugggacgaguucaucacuacuagacagcacaaauacgaacuucaaagaagagccagcugcuaaguaccaagcuaucuuugauaauaccacuagucugacggauaaacaccuggacccaaucagggaaaaucugggaaagcacuggaaaaacugugcccguaaacugggcuucacacagucucagauugaugaaauugaccaugacuaugagcgagauggacugaaagaaaagguuuaccagaugcuccaaaagugggugaugagggaaggcauaaagggagccacgguggggaagcuggcccaggcgcuccaccaguguuccaggaucgaccuucugagcagcuugauuuacgucagccagaacuaacccuggaugggcuacggcagcugaaguggacgccucacuuaguggauaaccccagaaaguuggcugccucagagcauucagaauucuguccucacugauagggguucugugucugcagaaauuuuguuuccuguacuucauagcuggagaauggggaaagaaaucugcagcaaaggggucucacucuguugccaggcuggucucaaacuucuggacucaagugauccucccgccucggccuuccaaagugcugggauaucaggcacugagccacugcgcccagccaacaauccgcucugaggaaagcguaagcaggaagaccucuuaauggcauagcaccaauaaaaaaaugacuccuaguuguguuuggaaagggagagaagagaugucugaggaaggucauguucuuucagcuuauggcauuuccuagaguuuuguugaagcaagaagaaaaacucagagaauauaaaaucaacuuuuaaaauugugugcucucuucuucacguaggcuccuguuaaaaacaaagugcagucagauucuaagcccuguucagagacuucguggaucacagcugcagcucaccgccacaucacaggauccguuaacguuaauacccaauacucugucagccacuguaggcucuaagaaccacgugcagucuucagcccauuaaauuaucgauuauuuuuuaaugaauugaauuuauauugagucuucaaauuaacugaauggauuuaaagggguaccaaggaggggggaaacaucagaauuucccaggcaguuguugcaaggaauugguacuaaccgugacuacaacaaaaauucuugauugacuuuuaaaguuauuuccuggcauucugguaccuucacccagccugagugcccuggagagggaacaggaaaugcugaucucuaccccugggugagaccagaaccucagggcugauacuguugaguggcuuccucgguuuacucuguguacugugaaaguauuuucauauuuuuucugugugccagagugaaaaaggacagcuucugagugugguaauugugccucuagcacccagccuuucaaagcccaccugaaaccuggggguggaugaaagaacuagaauagaagacugaagcuggguaggccgcucaguguccacuggcauuuugcuaaaccgacaaggaaggcugugugcuuagcucuccccagagggagggcgagaaggguguggugauggucaaucuggcugucggaacagauucuggugucuugggcugauaacaguguuguugauucugauugugaauccccucaacucuagcagacacauacacaccccugaaauggggcugcagagcaggcugucucagccuugccacugucggcaucucggccuggguaauucuguuguggggacuguccuguuccuuguaggauguuuaguagcaucccugcccccaccuacuagaugccaggggcacuguucuccccagccccccgccccaguugugacaauagucucuaaacauugucaaaugguccaaggaaaggggaaaauugccccgguugagaagagcacugcuguaaaguaaugagccucggcucuccugucugcaccuguccgguuacuacuuggccaccacgcagccuuggcuccuacagcccaaaagggagaauggagggaggcuccaggcuuugcuggaggggccugggugaguucuguuugcuccuuguaccaccauccaaaugguguuaucaaaucucuuagauuccaaagagguugaauaauuaauguucaaaggcaagagggcaaggcauuuuuuaacacuuuuuaaaauaaaaauuuauaccacaa

SEQ ID NO: 9 - RNA, 2477 nt, Homo sapiens
gugagccuggugguuggcagcuggagccacgucggagggggaagugucgcagcauucucugcaggcaucacagaccugaggcaguggccuccggagggcacuggacagaaacagccauccaaguggcugaguggagggacccugcucaagugcagcugcaguggccgggguuucccucagguagggaucggggcgccuugucgccgccagccacguguggcguccgguacagucagcagagugcagggugcgggcaccaggaaagggggcgcaggggaacucccgcgggccucgcguuugcaaacuucucgccugggcaggaggcggucgugggaaagaagguggaagagcgagcuuuuuggaacugugcacgggacagauuggacgcacaccccucgggaggcgcgaaggcauggaaaauuugaagcauauuaucacccuuggccaggucauccacaaacggugugaagagaugaaauacugcaagaaacagugccggcgccugggccaccgcguccucggccugaucaagccucuggagaugcuccaggaccaaggaaagaggagcgugcccucugagaaguuaaccacagccaugaaccgcuucaaggcugcccuggaggaggcuaauggggagauagaaaaguucagcaauagauccaauaucugcagguuucuaacagcaagccaggacaaaauacucuucaaggacgugaacaggaagcugagugaugucuggaaggagcucucgcuguuacuucagguugagcaacgcaugccuguuucacccauaagccaaggagcguccugggcacaggaagaucagcaggaugcagacgaagacaggcgagcuuuccagaugcuaagaagagauaaugaaaaaauagaagcuucacugagacgauuagaaaucaacaugaaagaaaucaaggaaacuuugaggcaguauuuaccaccaaaaugcaugcaggagaucccgcaagagcaaaucaaggagaucaagaaggagcagcuuucaggauccccguggauucugcuaagggaaaaugaagucagcacacuuuauaaaggagaauaccacagagcuccaguggccauaaaaguauucaaaaaacuccaggcuggcagcauugcaauagugaggcagacuuucaauaaggagaucaaaaccaugaagaaauucgaaucucccaacauccugcguauauuugggauuugcauugaugaaacagugacuccgccucaauucuccauugucauggaguacugugaacucgggacccugagggagcuguuggauagggaaaaagaccucacacuuggcaagcgcaugguccuaguccugggggcagcccgaggccuauaccggcuacaccauucagaagcaccugaacuccacggaaaaaucagaagcucaaacuuccugguaacucaaggcuaccaagugaagcuugcaggauuugaguugaggaaaacacagacuuccaugaguuugggaacuacgagagaaaagacagacagagucaaaucuacagcauaucucucaccucaggaacuggaagauguauuuuaucaauaugauguaaagucugaaauauacagcuuuggaaucguccucugggaaaucgccacuggagauaucccguuucaaggcuguaauucugagaagauccgcaagcugguggcugugaagcggcagcaggagccacugggugaagacugcccuucagagcugcgggagaucauugaugagugccgggcccaugaucccucugugcggcccucuguggaugaaaucuuaaagaaacucuccaccuuuucuaaguaguguaucaaaaucuaaaccaaggagucucuggacaagaagcugggagaggcacaaacuggacaucucucucucucauauccuucggcauuggguuaucuaugggugcaaggagugggcacgcuucucuguuacaaauagaaaacgauuccagucauacaggacacaucccacuccaaaugauauuuccaaaaacauaccucugacaguaacuuugauagaugguuugucaaauguaucuuucuggguauccacaccucuuggcaaugaaauuugcagcuccucccuuccauaaaugaagucucuuuccccaccauuugaaucugggcuggcacugugacuugauuugaucaauagaauguggaagaagugacuguaugccaguuccaagccuagguuucaagaggccuuauaaaugucuguuggaaccuuacccagccaugaacauguugagugagcaugcuggagaaugagagaccacaugaagcagaaacaugcuuuccuagcugaagucauacuagcccaaccaacauggcagcuaacacaugaaugaggccaaucaagaccagaagaaccacucaagcagaucccagcccaaauugcccauucacacaaucaggagcuaaauaaauuacuguugucuuaacacuaa

SEQ ID NO: 10 - RNA, 1853 nt, Homo sapiens
gugagccuggugguuggcagcuggagccacgucggagggggaagugucgcagcauucucugcaggcaucacagaccugaggcaguggccuccggagggcacuggacagaaacagccauccaaguggcugaguggagggacccugcucaagugcagcugcaguggccgggguuucccucagguagggaucggggcgccuugucgccgccagccacguguggcguccgguacagucagcagagugcagggugcgggcaccaggaaagggggcgcaggggaacucccgcgggccucgcguuugcaaacuucucgccugggcaggaggcggucgugggaaagaagguggaagagcgagcuuuuuggaacugugcacgggacagauuggacgcacaccccucgggaggcgcgaaggcauggaaaauuugaagcauauuaucacccuuggccaggucauccacaaacggugugaagagaugaaauacugcaagaaacagugccggcgccugggccaccgcguccucggccugaucaagccucuggagaugcuccaggaccaaggaaagaggagcgugcccucugagaaguuaaccacagccaugaaccgcuucaaggcugcccuggaggaggcuaauggggagauagaaaaguucagcaauagauccaauaucugcagguuucuaacagcaagccaggacaaaauacucuucaaggacgugaacaggaagcugagugaugucuggaaggagcucucgcuguuacuucagguugagcaacgcaugccuguuucacccauaagccaaggagcguccugggcacaggaagaucagcaggaugcagacgaagacaggcgagcuuuccagaugcuaagaagagauaaugaaaaaauagaagcuucacugagacgauuagaaaucaacaugaaagaaaucaaggaaacuuugaggcagucuuuggaaucguccucugggaaaucgccacuggagauaucccguuucaaggugaagaaugugaagacuggcucagccaguggcuguaauucugagaagauccgcaagcugguggcugugaagcggcagcaggagccacugggugaagacugcccuucagagcugcgggagaucauugaugagugccgggcccaugaucccucugugcggcccucuguggaugaaaucuuaaagaaacucuccaccuuuucuaaguaguguaucaaaaucuaaaccaaggagucucuggacaagaagcugggagaggcacaaacuggacaucucucucucucauauccuucggcauuggguuaucuaugggugcaaggagugggcacgcuucucuguuacaaauagaaaacgauuccagucauacaggacacaucccacuccaaaugauauuuccaaaaacauaccucugacaguaacuuugauagaugguuugucaaauguaucuuucuggguauccacaccucuuggcaaugaaauuugcagcuccucccuuccauaaaugaagucucuuuccccaccauuugaaucugggcuggcacugugacuugauuugaucaauagaauguggaagaagugacuguaugccaguuccaagccuagguuucaagaggccuuauaaaugucuguuggaaccuuacccagccaugaacauguugagugagcaugcuggagaaugagagaccacaugaagcagaaacaugcuuuccuagcugaagucauacuagcccaaccaacauggcagcuaacacaugaaugaggccaaucaagaccagaagaaccacucaagcagaucccagcccaaauugcccauucacacaaucaggagcuaaauaaauuacuguugucuuaacacuaa

SEQ ID NO: 11 - RNA, 1899 nt, Mus musculus
ggagccucuuauuugaaagggaaguugugaagucgcucauucaaaguccgggggagcgaccugggaagcggaauucggggccugaaccuccgugccuugaccuacugauuguucccuuugcagacccugccuuccucucaguccacacuccgagccguagacgucggguuuccagcuccgucugugcacacauaacuccaggaagccgcuagcucccgacgaugucuucugucaaguuauggccuacuggugcgucagcgguuccucuggugagccgugaagaacugaagaagcuggaguuuguggguaaaggaggguucggagucguguuccgggcacaccacagaacauggaaccaugauguagcagucaagaucgugaacucgaagaagauauccugggaggugaaggcuaugguuaaucuucguaaugagaacguucugcuccugcugggggucacugaggaccuccagugggacuucguguccgggcaggcucuggugacaagauucauggagaauggcucccucgcagggcugcugcaacccgagugcccucggcccuggccacuccucugucgccugcugcaggaaguggugcuggggaugugcuaccuacacagcuugaacccuccgcuccugcaccgggaccucaagcccucuaacauucugcuggauccagagcuccacgccaagcuagcagauuuuggccuguccacguuucagggagggucccagucagggucaggaucaggaucaggauccagggacucugggggcacccuagcguacuuggacccagagcuguuauuugaugucaaccugaaggcuucuaaagcgagugaugucuacagcuuugggauccucgugugggcagugcuggcuggcagagaagcugaguugguagacaagacuucacuaauccgggaaacagugugugacaggcagagucguccuccacugacagagcugccuccagguagcccugagacucccggcuuggaaaaacugaaggaguuaaugauucauugcugggguucccaguccgaaaacaggccauccuuccaggacugcgaaccaaaaaccaaugaaguuuacaaucugguaaaggacaagguagaugcugcugucuccgagguaaagcauuaucugucucagcacagaagcagcggcagaaacuugucugccagagagccaagccaaagaggcacagaaauggauugcccgagggaaaccaugguuucuaaaaugcuggaccgccugcauuuggaggaacccuccggaccaguuccuggaaaauguccugagaggcaagcacaggacacaucaguugggccugccacaccagcaaggacaucuucugaccccguggcuggcacuccucagauuccacauacuuuacccuucagaggcacaacaccugggccagucuuuacugagacucccgguccucacccccaaaggaaucagggagauggaagacacggcacuccuugguaucccuggaccccaccgaauccaaugacagggccaccggcucucgucuucaacaacuguucugaagugcagauugggaacuacaacuccuugguagcaccaccaagaacuacugccucaaguucggccaaguaugaccaagcacaguucggcagggguaggggcuggcagcccuuccacaaguagacuucagagaaucacugcaagagccugaagugugccauucagcguggcaauaaaaagcacguuuuaagcaaccuggacuggcuaagacaguccuugccacuuccugaagcucacaacauucugugaggacaguuggaccuacacccaaacugacucuugacccaucuccuuaaagucaauaaacauagcauguuaacugugaaaaaaaaaaaaaaaa

SEQ ID NO: 12 - RNA, 1882 nt, Mus musculus
ggagccucuuauuugaaagggaaguugugaagucgcucauucaaaguccgggggagcgaccugggaagcggaauucggggccugaaccuccgugccuugaccuacugauuguucccuuugcagacccugccuuccucucaguccacacuccgagccguagacgucggguuuccagcuccgucugugcacacauaacuccaggaagccgcuagcucccgacgaugucuucugucaaguuaugcgguuccucuggugagccgugaagaacugaagaagcuggaguuuguggguaaaggaggguucggagucguguuccgggcacaccacagaacauggaaccaugauguagcagucaagaucgugaacucgaagaagauauccugggaggugaaggcuaugguuaaucuucguaaugagaacguucugcuccugcugggggucacugaggaccuccagugggacuucguguccgggcaggcucuggugacaagauucauggagaauggcucccucgcagggcugcugcaacccgagugcccucggcccuggccacuccucugucgccugcugcaggaaguggugcuggggaugugcuaccuacacagcuugaacccuccgcuccugcaccgggaccucaagcccucuaacauucugcuggauccagagcuccacgccaagcuagcagauuuuggccuguccacguuucagggagggucccagucagggucaggaucaggaucaggauccagggacucugggggcacccuagcguacuuggacccagagcuguuauuugaugucaaccugaaggcuucuaaagcgagugaugucuacagcuuugggauccucgugugggcagugcuggcuggcagagaagcugaguugguagacaagacuucacuaauccgggaaacagugugugacaggcagagucguccuccacugacagagcugccuccagguagcccugagacucccggcuuggaaaaacugaaggaguuaaugauucauugcugggguucccaguccgaaaacaggccauccuuccaggacugcgaaccaaaaaccaaugaaguuuacaaucugguaaaggacaagguagaugcugcugucuccgagguaaagcauuaucugucucagcacagaagcagcggcagaaacuugucugccagagagccaagccaaagaggcacagaaauggauugcccgagggaaaccaugguuucuaaaaugcuggaccgccugcauuuggaggaacccuccggaccaguuccuggaaaauguccugagaggcaagcacaggacacaucaguugggccugccacaccagcaaggacaucuucugaccccguggcuggcacuccucagauuccacauacuuuacccuucagaggcacaacaccugggccagucuuuacugagacucccgguccucacccccaaaggaaucagggagauggaagacacggcacuccuugguaucccuggaccccaccgaauccaaugacagggccaccggcucucgucuucaacaacuguucugaagugcagauugggaacuacaacuccuugguagcaccaccaagaacuacugccucaaguucggccaaguaugaccaagcacaguucggcagggguaggggcuggcagcccuuccacaaguagacuucagagaaucacugcaagagccugaagugugccauucagcguggcaauaaaaagcacguuuuaagcaaccuggacuggcuaagacaguccuugccacuuccugaagcucacaacauucugugaggacaguuggaccuacacccaaacugacucuugacccaucuccuuaaagucaauaaacauagcauguuaacugugaaaaaaaaaaaaaaaa

SEQ ID NO: 13 - RNA, 1980 nt, Mus musculus
ggagccucuuauuugaaagggaaguugugaagucgcucauucaaaguccgggggagcgaccugggaagcggaauucggggccugaaccuccgugccuugaccuacugauuguucccuuugcagacccugccuuccucucaguccacacuccgagccguagacgucggguuuccagcuccgucugugcacacauaacuccaggaagccgcuagcucccgacgaugucuucugucaaguuaugucacggaaucccggagcuugggagacagaggaugcgcuaggcugggggaugggcccccuaccggacaagaaccuuucucaggccuacuggugcgucagcgguuccucuggugagccgugaagaacugaagaagcuggaguuuguggguaaaggaggguucggagucguguuccgggcacaccacagaacauggaaccaugauguagcagucaagaucgugaacucgaagaagauauccugggaggugaaggcuaugguuaaucuucguaaugagaacguucugcuccugcugggggucacugaggaccuccagugggacuucguguccgggcaggcucuggugacaagauucauggagaauggcucccucgcagggcugcugcaacccgagugcccucggcccuggccacuccucugucgccugcugcaggaaguggugcuggggaugugcuaccuacacagcuugaacccuccgcuccugcaccgggaccucaagcccucuaacauucugcuggauccagagcuccacgccaagcuagcagauuuuggccuguccacguuucagggagggucccagucagggucaggaucaggaucaggauccagggacucugggggcacccuagcguacuuggacccagagcuguuauuugaugucaaccugaaggcuucuaaagcgagugaugucuacagcuuugggauccucgugugggcagugcuggcuggcagagaagcugaguugguagacaagacuucacuaauccgggaaacagugugugacaggcagagucguccuccacugacagagcugccuccagguagcccugagacucccggcuuggaaaaacugaaggaguuaaugauucauugcugggguucccaguccgaaaacaggccauccuuccaggacugcgaaccaaaaaccaaugaaguuuacaaucugguaaaggacaagguagaugcugcugucuccgagguaaagcauuaucugucucagcacagaagcagcggcagaaacuugucugccagagagccaagccaaagaggcacagaaauggauugcccgagggaaaccaugguuucuaaaaugcuggaccgccugcauuuggaggaacccuccggaccaguuccuggaaaauguccugagaggcaagcacaggacacaucaguugggccugccacaccagcaaggacaucuucugaccccguggcuggcacuccucagauuccacauacuuuacccuucagaggcacaacaccugggccagucuuuacugagacucccgguccucacccccaaaggaaucagggagauggaagacacggcacuccuugguaucccuggaccccaccgaauccaaugacagggccaccggcucucgucuucaacaacuguucugaagugcagauugggaacuacaacuccuugguagcaccaccaagaacuacugccucaaguucggccaaguaugaccaagcacaguucggcagggguaggggcuggcagcccuuccacaaguagacuucagagaaucacugcaagagccugaagugugccauucagcguggcaauaaaaagcacguuuuaagcaaccuggacuggcuaagacaguccuugccacuuccugaagcucacaacauucugugaggacaguuggaccuacacccaaacugacucuugacccaucuccuuaaagucaauaaacauagcauguuaacugugaaaaaaaaaaaaaaaa

SEQ ID NO: 14 - RNA, 4512 nt, Mus musculus
cgaaaagcgcggaacuugcugucaucuagcgggagguuggacucuucuugaggucguuuuagcucaagucgagacugaaggacacagcacuaagcaagaaccaaaagugguguguuggagauucugagcaaucaaaaugcaaccagacauguccuuggacaauauuaagauggcauccagugaccugcuggagaagacagaccuagacagcggaggcuucgggaagguguccuuguguuaccacagaagccauggauuugucauccugaaaaaaguauacacagggcccaaccgcgcugaguacaaugagguucucuuggaagaggggaagaugaugcacagacugagacacagucgaguggugaagcuacugggcaucaucauagaagaagggaacuauucgcuggugauggaguacauggagaagggcaaccugaugcacgugcuaaagacccagauagaugucccacuuucauugaaaggaaggauaaucguggaggccauagaaggcaugugcuacuuacaugacaaaggugugauacacaaggaccugaagccugagaauauccucguugaucgugacuuucacauuaagauagccgaucuugguguggcuuccuuuaagacauggagcaaacugacuaaggagaaagacaacaagcagaaagaagugagcagcaccacuaagaagaacaauggugguacccuuuacuacauggcacccgaacaccugaaugacaucaaugcaaagcccacggagaagucggacguguacagcuuuggcauuguccuuugggcaauauuugcaaaaaaggagcccuaugagaaugucaucuguacugagcaguucgugaucugcauaaaaucugggaacaggccaaauguagaggaaauccuugaguacuguccaagggagaucaucagccucauggagcggugcuggcaggcgaucccagaagacaggccaacauuucuuggcauugaagaagaauuuaggccuuuuuacuuaagucauuuugaagaauauguagaagaggauguggcaaguuuaaagaaagaguauccagaucaaagcccagugcugcagagaauguuuucacugcagcaugacuguguacccuuaccuccgagcaggucaaauucagaacaaccuggaucgcugcacaguucccaggggcuccagauggguccuguggaggaguccugguuuucuuccuccccagaguacccacaggacgagaaugaucgcagugugcaggcuaagcugcaagaggaagccagcuaucaugcuuuuggaauauuugcagagaaacagacaaaaccgcagccaaggcagaaugaggcuuacaacagagaggaggaaaggaaacgaagggucucucaugaccccuuugcacagcagagagcucgugagaauauuaagagugcaggagcaagaggucauucugaucccagcacaacgagucguggaauugcagugcaacagcugucauggccagccacccaaacaguuuggaacaauggauuguauaaucagcauggauuuggaacuacagguacaggaguuugguauccgccaaaucuaagccaaauguauaguacuuauaaaacuccagugccagagaccaacauaccgggaagcacacccaccaugccauacuucucugggccaguagcagaugaccucauaaaauauacuauauucaauaguucugguauucagauuggaaaccacaauuauauggauguuggacugaauucacaaccaccaaacaauacuugcaaagaagagucgacuuccagacaccaagccaucuuugauaacaccacuagucugacugaugaacaccugaacccuaucagggaaaaccugggaaggcaguggaaaaacugugcccgcaagcugggcuucacugagucucagaucgaugaaaucgaccaugacuaugaaagagauggacugaaagagaaaguuuaccaaaugcuucagaaguggcugaugcgggaaggcaccaaaggggccacagugggaaaguuggcccaggcacuucaccaauguugcaggauagaccugcugaaccacuugauucgugccagccagagcuaagccugggcaggcucuggcagugggaagcaaacuauuugucuggugcacaaaccccguuugcccacuagccuucagaacucuaucucagcaugagcucugcauuugagcacacagggucaugcaguuuggaacugguggaugggaagagaaaucugaagcccacagugauucuucagaacaugcaagcauaaagaccgcugaaugaauggucgguccaugaccaguaggaggaaaaaaauuaaaaauacaguguauugaguuuucaaagggagagaagauguugggggaagguggccuucguucagcuugugucauagucaucacuuagguuauuugcucaguuuccugugguuucauuggguaaggggaaaacaaacaaaaaacauucaaagaauguaaaaucagcuagcuucccgcuucauggacugugagucagaaaaguucugcucaaaugccuucuaugagccacggcucuucuaaaaacuauaggaucuguuaacuugagcauccaaugcuguacugucaccuuaagcuuuaagaaucauguguaauucuagucuaucaaauuaucaauuagugagagauuuuuaaggagugcuaaggagaaaaaggugcagggcagagagagaucuucagguacuggcugucauugcagcucagguacuggcugucauugcagagcugcugcugguucuggcugcuguaaaaagucuacuuuuaaguuucuaaaguuucucuaccauucuggcgcuucaugcaguuucccacccugugagccccacagagggaacagaugugcaggaaacacugaucgggaaauaccaucuccuccucucuggauaugacuggugccuuagcacuguucccugugcccaauaaacuguuuccucaguuuucuccagugugcuguacaaauauguuucuucugugugccaccaaucaagaaaggacagcuucagagucaucuugugccuuuaggaagcccauauugcaaccuacaagugagugaaggagcuaggguagaagacugaagccgccuacaccagccacagucacuguuaggaugggcugcacagccgcuuuggccuuguuggccauucuggcacucauuggcacuucauccuccuuuguugggcuauccuguacucaguaggauauuugggaacauuccuggccuccaucugcaagaugccaacaguacaagucaaccucacaacacccaccaggugugagaaaaauaacuagauauugcuacauguucuuuggagggacucuccuguaacguuauuucccccauccccagacaagguuucucaguucucgcuguccuggaacucacuuuguagaccagauuggucucccaauucagagaucugccugccucugccuccugagugcugggaauaaaggcaugugccaccaccacccagcuuccugcuguaaguuuauaugccuuacuucuccugucacacaggcuaggccaucauuaacagcauggaacaugcacaguggaagggaauuggagcagggaagguuccaggcuuuccugaaggagccugcauagucuuuaaaaaauguuuucuuuuuaacuucuaaugcuguuuuaagaagacagaauuagaaacugcuucuaguggaguuuuaaggagaauaaaauagcaagggaggccagagcugguuuuggaauugcaguagcaaucaggguccuaguauuaaaaucaugucuguuaguguucaguaggggaagacuuguguguccacugcugaagauaggugcuucagauccaggccuggauugugaccacagacuuucagaucuuuuccaggcucaccacugacagccagacaauggcuuggcaccucgaguuguggcugagccuguaugaguugaaggauggucaguagaugaagccguuuacucucauggugugugcccuuguacaagagaucugggcgcgugacacaaagccagucauugacaggcuagacccaaagaccuuuaagcacucccauuugcuuccgcuguaucacaauaacugaugacuuuguagucagugguaccugcuccgaacaaguacguggagugugggagucccucugggagcccagcguggaauuagaacaccucuuuaaagccaccuccuaagccgagcagaaugcaguggacugggguacaagucagaccugagugucauuguccacaucauggaaaaacaagauggccaccagaacacugaaggccugaggagacuaacccuguucccagaacucagaguucuguuugguucuguuuuuaauaagauaaccuuucuuuaauguauacauaugcauauacauauauauuuguacuuuaaauacagaguacugaauaaaauuuaugugacuauaaaaaaaaaaaaaaaaaa

SEQ ID NO: 15 - RNA, 4759 nt, Mus musculus
ggagacaggggcggccgcagccuagcaccaccccaguaguccuuagaggaggaccagcggcccccggaacggccucgggagcgcgggugucggugccgcucaccucccgggaguccagcggcggugcggacccgcggaggugcgagcuggacgcggcgggaggcggcaggcugugggggaccugcgaguggcugggcgccacuucagcaccgaacuccgaccgguccucgucagggcccagggccgucccggccgggcagccacggcgaggcgccgcuccugacguccaccgcccguccuuccucaccuaggccgggguacuuggccgcgggaaggccucugaggacacagcacuaagcaagaaccaaaagugguguguuggagauucugagcaaucaaaaugcaaccagacauguccuuggacaauauuaagauggcauccagugaccugcuggagaagacagaccuagacagcggaggcuucgggaagguguccuuguguuaccacagaagccauggauuugucauccugaaaaaaguauacacagggcccaaccgcgcugaguacaaugagguucucuuggaagaggggaagaugaugcacagacugagacacagucgaguggugaagcuacugggcaucaucauagaagaagggaacuauucgcuggugauggaguacauggagaagggcaaccugaugcacgugcuaaagacccagauagaugucccacuuucauugaaaggaaggauaaucguggaggccauagaaggcaugugcuacuuacaugacaaaggugugauacacaaggaccugaagccugagaauauccucguugaucgugacuuucacauuaagauagccgaucuugguguggcuuccuuuaagacauggagcaaacugacuaaggagaaagacaacaagcagaaagaagugagcagcaccacuaagaagaacaauggugguacccuuuacuacauggcacccgaacaccugaaugacaucaaugcaaagcccacggagaagucggacguguacagcuuuggcauuguccuuugggcaauauuugcaaaaaaggagcccuaugagaaugucaucuguacugagcaguucgugaucugcauaaaaucugggaacaggccaaauguagaggaaauccuugaguacuguccaagggagaucaucagccucauggagcggugcuggcaggcgaucccagaagacaggccaacauuucuuggcauugaagaagaauuuaggccuuuuuacuuaagucauuuugaagaauauguagaagaggauguggcaaguuuaaagaaagaguauccagaucaaagcccagugcugcagagaauguuuucacugcagcaugacuguguacccuuaccuccgagcaggucaaauucagaacaaccuggaucgcugcacaguucccaggggcuccagauggguccuguggaggaguccugguuuucuuccuccccagaguacccacaggacgagaaugaucgcagugugcaggcuaagcugcaagaggaagccagcuaucaugcuuuuggaauauuugcagagaaacagacaaaaccgcagccaaggcagaaugaggcuuacaacagagaggaggaaaggaaacgaagggucucucaugaccccuuugcacagcagagagcucgugagaauauuaagagugcaggagcaagaggucauucugaucccagcacaacgagucguggaauugcagugcaacagcugucauggccagccacccaaacaguuuggaacaauggauuguauaaucagcauggauuuggaacuacagguacaggaguuugguauccgccaaaucuaagccaaauguauaguacuuauaaaacuccagugccagagaccaacauaccgggaagcacacccaccaugccauacuucucugggccaguagcagaugaccucauaaaauauacuauauucaauaguucugguauucagauuggaaaccacaauuauauggauguuggacugaauucacaaccaccaaacaauacuugcaaagaagagucgacuuccagacaccaagccaucuuugauaacaccacuagucugacugaugaacaccugaacccuaucagggaaaaccugggaaggcaguggaaaaacugugcccgcaagcugggcuucacugagucucagaucgaugaaaucgaccaugacuaugaaagagauggacugaaagagaaaguuuaccaaaugcuucagaaguggcugaugcgggaaggcaccaaaggggccacagugggaaaguuggcccaggcacuucaccaauguugcaggauagaccugcugaaccacuugauucgugccagccagagcuaagccugggcaggcucuggcagugggaagcaaacuauuugucuggugcacaaaccccguuugcccacuagccuucagaacucuaucucagcaugagcucugcauuugagcacacagggucaugcaguuuggaacugguggaugggaagagaaaucugaagcccacagugauucuucagaacaugcaagcauaaagaccgcugaaugaauggucgguccaugaccaguaggaggaaaaaaauuaaaaauacaguguauugaguuuucaaagggagagaagauguugggggaagguggccuucguucagcuugugucauagucaucacuuagguuauuugcucaguuuccugugguuucauuggguaaggggaaaacaaacaaaaaacauucaaagaauguaaaaucagcuagcuucccgcuucauggacugugagucagaaaaguucugcucaaaugccuucuaugagccacggcucuucuaaaaacuauaggaucuguuaacuugagcauccaaugcuguacugucaccuuaagcuuuaagaaucauguguaauucuagucuaucaaauuaucaauuagugagagauuuuuaaggagugcuaaggagaaaaaggugcagggcagagagagaucuucagguacuggcugucauugcagcucagguacuggcugucauugcagagcugcugcugguucuggcugcuguaaaaagucuacuuuuaaguuucuaaaguuucucuaccauucuggcgcuucaugcaguuucccacccugugagccccacagagggaacagaugugcaggaaacacugaucgggaaauaccaucuccuccucucuggauaugacuggugccuuagcacuguucccugugcccaauaaacuguuuccucaguuuucuccagugugcuguacaaauauguuucuucugugugccaccaaucaagaaaggacagcuucagagucaucuugugccuuuaggaagcccauauugcaaccuacaagugagugaaggagcuaggguagaagacugaagccgccuacaccagccacagucacuguuaggaugggcugcacagccgcuuuggccuuguuggccauucuggcacucauuggcacuucauccuccuuuguugggcuauccuguacucaguaggauauuugggaacauuccuggccuccaucugcaagaugccaacaguacaagucaaccucacaacacccaccaggugugagaaaaauaacuagauauugcuacauguucuuuggagggacucuccuguaacguuauuucccccauccccagacaagguuucucaguucucgcuguccuggaacucacuuuguagaccagauuggucucccaauucagagaucugccugccucugccuccugagugcugggaauaaaggcaugugccaccaccacccagcuuccugcuguaaguuuauaugccuuacuucuccugucacacaggcuaggccaucauuaacagcauggaacaugcacaguggaagggaauuggagcagggaagguuccaggcuuuccugaaggagccugcauagucuuuaaaaaauguuuucuuuuuaacuucuaaugcuguuuuaagaagacagaauuagaaacugcuucuaguggaguuuuaaggagaauaaaauagcaagggaggccagagcugguuuuggaauugcaguagcaaucaggguccuaguauuaaaaucaugucuguuaguguucaguaggggaagacuuguguguccacugcugaagauaggugcuucagauccaggccuggauugugaccacagacuuucagaucuuuuccaggcucaccacugacagccagacaauggcuuggcaccucgaguuguggcugagccuguaugaguugaaggauggucaguagaugaagccguuuacucucauggugugugcccuuguacaagagaucugggcgcgugacacaaagccagucauugacaggcuagacccaaagaccuuuaagcacucccauuugcuuccgcuguaucacaauaacugaugacuuuguagucagugguaccugcuccgaacaaguacguggagugugggagucccucugggagcccagcguggaauuagaacaccucuuuaaagccaccuccuaagccgagcagaaugcaguggacugggguacaagucagaccugagugucauuguccacaucauggaaaaacaagauggccaccagaacacugaaggccugaggagacuaacccuguucccagaacucagaguucuguuugguucuguuuuuaauaagauaaccuuucuuuaauguauacauaugcauauacauauauauuuguacuuuaaauacagaguacugaauaaaauuuaugugacuaua

SEQ ID NO: 16 - RNA, 2031 nt, Mus musculus
cggagcagaacucuggacagaaauagcugucggacagguugguuuugaggacucugcuggagcauugcugcguuggcccaaauuugacuuuaggcgggaaccgcggcuggugcggaguuccuccgagaggcgagagcuuuaagguugcaccaagggaggggacucaggacaccggggaccugcuccgaccuggcgugcauugucaacgaggcagaagaccgcgguccgggagcggcgggagacugaugauccuacaggaacugagcgcaggauagaccggacgagcgccccucggagaucacguagaauccagugagcuguagcgcggcaggaggaagcaggagaaucccagaaucacagauuggacuuccuggaacuuaggcuauggauaaauugggacagaucaucaaguuaggccagcucaucuaugaacagugugaaaagaugaaauacugccggaaacaaugccagcgucuaggaaaccgugugcacggccugcuacagccucuccagagacuccaggcccaaggaaagaagaaccugcccgaugacauuacugcugcccugggccguuuugaugaaguccugaaggaggcuaaccagcagauagaaaaguucagcaagaagucccauauuuggaaguuugugagugugggcaaugauaagauccucuuccaugaagugaaugagaagcugagagacgucugggaggagcuguugcugcugcuucagguuuaucauuggaauaccguuucagaugucagccagccagcauccuggcagcaggaagaucgacaggaugcagaggaagacggaaaugaaaauaugaaaguuauccugaugcaguugcaaauuagcguggaagaaaucaacaaaacccugaagcaaugcucacuaaaacccacacaggagaucccacaagaucuccaaaucaaggagauuccaaaggaacaucuuggaccuccguggaccaaacugaagacaaguaaaaugagcaccauuuauagaggagaguaucacagaucuccaguuaccaucaaaguauucaacaacccccaggccgaaaguguuggaauagugagguucacuuucaaugacgagaucaaaaccaugaagaaauucgauucucccaacaucuugcguauauuugggauuugcauugaucaaacagugaagcccccugaguucuccauugucauggaguacugugaacuuggaacccugagggaacugcuggauagagaaaaagaccucacaaugagugugcgcagccuccuaguccugagggcagccagaggcuuauacaggcuacaccauucggaaacacuccacagaaacaucagcagcuccaguuuccucguagccggaggcuaccaaguaaagcuugcaggauuugaguuaagcaaaacacagaauuccaucagccggacagcaaagagcacuaaagcagagagauccaguucaacgauauaugucuccccugagagacugaaaaauccauuuugccuuuaugacauaaaagcugaaauauauagcuuuggaauuguacucugggaaauugccacuggaaagaucccauuugaaggcugugauucuaagaagauccgggagcugguggcugaggacaagaagcaggaaccagugggucaggauugcccugaguuguugcgggaaaucauuaaugagugucgugcccaugagcccucccaacggcccucuguggacgguaggagucuuucuggcagagaacgaaucuuggagagacugucugcgguugaagaauccacggacaagaagguguaagaggacaaaguggacauuuguccuucauaucuguuagcagcaucaacuuauugcugggagcaaauagcuucuuucucccuugcuugcuugcuuuuuuucuuucuuuuacuugagaugggaucucaccauguagccccugaaaacuguguagaacaggcuggcucaaacucaaggaaaucuuccaguucugccuccugaaugcuggaauuaaaggcauagaccaccaaa

SEQ ID NO: 17 - RNA, 2007 nt, Mus musculus
cggagcagaacucuggacagaaauagcugucggacagguugguuuugaggacucugcuggagcauugcugcguuggcccaaauuugacuuuaggcgggaaccgcggcuggugcggaguuccuccgagaggcgagagcuuuaagguugcaccaagggaggggacucaggacaccggggaccugcuccgaccuggcgugcauugucaacgaggcagaagaccgcgguccgggagcggcgggagacugaugauccuacaggaacugagcgcaggauagaccggacgagcgccccucggagaucacguagaauccagugagcuguagcgcggcaggaggaagcaggagaaucccagaaucacagauuggacuuccuggaacuuaggcuauggauaaauugggacagaucaucaaguuaggccagcucaucuaugaacagugugaaaagaugaaauacugccggaaacaaugccagcgucuaggaaaccgugugcacggccugcuacagccucuccagagacuccaggcccaaggaaagaagaaccugcccgaugacauuacugcugcccugggccguuuugaugaaguccugaaggaggcuaaccagcagauagaaaaguucagcaagaagucccauauuuggaaguuugugagugugggcaaugauaagauccucuuccaugaagugaaugagaagcugagagacgucugggaggagcuguugcugcugcuucagguuuaucauuggaauaccguuucagaugucagccagccagcauccuggcagcaggaagaucgacaggaugcagaggaagacggaaaugaaaauaugaaaguuauccugaugcaguugcaaauuagcguggaagaaaucaacaaaacccugaagcaaugcucacuaaaacccacacaggagaucccacaagaucuccaaaucaaggagauuccaaaggaacaucuuggaccuccguggaccaaacugaagacaaguaaaaugagcaccauuuauagaggagaguaucacagaucuccaguuaccaucaaaguauucaacaacccccaggccgaaaguguuggaauagugagguucacuuucaaugacgagaucaaaaccaugaagaaauucgauucucccaacaucuugcguauauuugggauuugcauugaucaaacagugaagcccccugaguucuccauugucauggaguacugugaacuuggaacccugagggaacugcuggauagagaaaaagaccucacaaugagugugcgcagccuccuaguccugagggcagccagaggcuuauacaggcuacaccauucggaaacacuccacagaaacaucagcagcuccaguuuccucguagccggaggcuaccaaguaaagcuugcaggauuugaguuaagcaaaacacagaauuccaucagccggacagcaaagagcacuaaagcagagagauccaguucaacgauauaugucuccccugagagacugaaaaauccauuuugccuuuaugacauaaaagcugaaauauauagcuuuggaauuguacucugggaaauugccacuggaaagaucccauuugaaggcugugauucuaagaagauccgggagcugguggcugaggacaagaagcaggaaccagugggucaggauugcccugaguuguugcgggaaaucauuaaugagugucgugcccaugagcccucccaacggcccucuguggacggaaucuuggagagacugucugcgguugaagaauccacggacaagaagguguaagaggacaaaguggacauuuguccuucauaucuguuagcagcaucaacuuauugcugggagcaaauagcuucuuucucccuugcuugcuugcuuuuuuucuuucuuuuacuugagaugggaucucaccauguagccccugaaaacuguguagaacaggcuggcucaaacucaaggaaaucuuccaguucugccuccugaaugcuggaauuaaaggcauagaccaccaaa

SEQ ID NO: 18 - RNA, 19 nt, Artificial Sequence
  Exemplary target mRNA sequence
gaugucgugcgucaaguua

SEQ ID NO: 19 - RNA, 19 nt, Artificial Sequence
  Exemplary target mRNA sequence
ggcaagucuggauaacgaa

SEQ ID NO: 20 - RNA, 19 nt, Artificial Sequence
  Exemplary target mRNA sequence
gcugcugucuccacgguaa

SEQ ID NO: 21 - RNA, 19 nt, Artificial Sequence
  Exemplary target mRNA sequence
gaaugucauuaagaugaaa

SEQ ID NO: 22 - RNA, 19 nt, Artificial Sequence
  Exemplary target mRNA sequence
gggcgauauuugcaaauaa

SEQ ID NO: 23 - RNA, 19 nt, Artificial Sequence
  Exemplary target mRNA sequence
cgugaagaguuuaaagaaa

SEQ ID NO: 24 - RNA, 19 nt, Artificial Sequence
  Exemplary target mRNA sequence
ggugugaagagaugaaaua

SEQ ID NO: 25 - RNA, 19 nt, Artificial Sequence
  Exemplary target mRNA sequence
ggagaucccgcaagagcaa

SEQ ID NO: 26 - RNA, 19 nt, Artificial Sequence
  Exemplary target mRNA sequence
ggaugaaaucuuaaagaaa

SEQ ID NO: 27 - RNA, 19 nt, Artificial Sequence
  Exemplary target mRNA sequence
gguagacaagacuucacua

SEQ ID NO: 28 - RNA, 19 nt, Artificial Sequence
  Exemplary target mRNA sequence
gaugucuucugucaaguua

SEQ ID NO: 29 - RNA, 19 nt, Artificial Sequence
  Exemplary target mRNA sequence
gugaacucgaagaagauau

SEQ ID NO: 30 - RNA, 19 nt, Artificial Sequence
  Exemplary target mRNA sequence
ggaagguguccuuguguua

SEQ ID NO: 31 - RNA, 19 nt, Artificial Sequence
  Exemplary target mRNA sequence
gugagcagcaccacuaaga

SEQ ID NO: 32 - RNA, 19 nt, Artificial Sequence
  Exemplary target mRNA sequence
gaaagaguauccagaucaa

SEQ ID NO: 33 - RNA, 19 nt, Artificial Sequence
  Exemplary target mRNA sequence
gggacagaucaucaaguua

SEQ ID NO: 34 - RNA, 19 nt, Artificial Sequence
  Exemplary target mRNA sequence
gaggaagacggaaaugaaa

SEQ ID NO: 35 - RNA, 19 nt, Artificial Sequence
  Exemplary target mRNA sequence
gcagagagauccaguucaa

SEQ ID NO: 36 - DNA, 21 bp, Artificial Sequence
  RT-PCR forwards primer
ggcccggtgagagactccaca

SEQ ID NO: 37 - DNA, 20 bp, Artificial Sequence
  RT-PCR forwards primers
tgatcagtcaacgggggaca

SEQ ID NO: 38 - DNA, 28 bp, Artificial Sequence
  RT-PCR forwards primer
gcatctctgagtctcaaattatgacttg

SEQ ID NO: 39 - DNA, 18 bp, Artificial Sequence
  RT-PCR forwards primer
cacccaccccagcaagga

SEQ ID NO: 40 - DNA, 21 bp, Artificial Sequence
  RT-PCR forwards primer
atcaaagtattcaacaacccc

SEQ ID NO: 41 - DNA, 20 bp, Artificial Sequence
  RT-PCR forwards primer
agaagaagggaactattcgc

SEQ ID NO: 42 - DNA, 19 bp, Artificial Sequence
  RT-PCR forwards primer
cgggcacaccacagaacat

SEQ ID NO: 43 - DNA, 21 bp, Artificial Sequence
  RT-PCR forwards primer
attatgagaagtgcctcatgg

SEQ ID NO: 44 - DNA, 22 bp, Artificial Sequence
  RT-PCR forwards primer
tcctccatgaacaaacagttcc

SEQ ID NO: 45 - DNA, 20 bp, Artificial Sequence
  RT-PCR forwards primer
ggtagctgcaggagagcacg

SEQ ID NO: 46 - DNA, 20 bp, Artificial Sequence
  RT-PCR forwards primer
tgctcgacatcacagagcag

SEQ ID NO: 47 - DNA, 19 bp, Artificial Sequence
  RT-PCR forwards primer
agagcgcggttacagcttc

SEQ ID NO: 48 - DNA, 30 bp, Artificial Sequence
  RT-PCT reverse primer
ccaggagatgtaactctaatccaggtttgc

SEQ ID NO: 49 - DNA, 20 bp, Artificial Sequence
  RT-PCT reverse primer
tccaacacttcgagaggtcc

SEQ ID NO: 50 - DNA, 23 bp, Artificial Sequence
  RT-PCT reverse primer
gcgttctgctccttctagcttct

SEQ ID NO: 51 - DNA, 20 bp, Artificial Sequence
  RT-PCT reverse primer
atgggggtctgggatggaaa

SEQ ID NO: 52 - DNA, 21 bp, Artificial Sequence
  RT-PCT reverse primer
gcaaatcccaaatatacgcaa

SEQ ID NO: 53 - DNA, 18 bp, Artificial Sequence
  RT-PCT reverse primer
ttctatggcctccacgat

SEQ ID NO: 54 - DNA, 21 bp, Artificial Sequence
  RT-PCT reverse primer
gtagcacatccccagcaccac

SEQ ID NO: 55 - DNA, 20 bp, Artificial Sequence
  RT-PCT reverse primer
gacattctggttcagggttc

SEQ ID NO: 56 - DNA, 21 bp, Artificial Sequence
  RT-PCT reverse primer
aatcatggacgactccttggg

SEQ ID NO: 57 - DNA, 20 bp, Artificial Sequence
  RT-PCT reverse primer
ctggggacaggattcgtctc

SEQ ID NO: 58 - DNA, 20 bp, Artificial Sequence
  RT-PCT reverse primer
gatctgcagacacacactgg

SEQ ID NO: 59 - DNA, 22 bp, Artificial Sequence
  RT-PCT reverse primer
ctccaccttgtgaacactcctt

SEQ ID NO: 60 - DNA, 1044 bp, Artificial Sequence
  Exemplary expression construct (5′ to 3′) encoding Ripk3 shRNAs operably linked to pol III promoters
gctagcaattgggatccaattcgaacgctgacgtcatcaacccgctccaaggaatcgcgggcccagtgtcactaggcgggaacacccagcgcgcgtgcgccctggcaggaagatggctgtgagggacaggggagtggcgccctgcaatatttgcatgtcgctatgtgttctgggaaatcaccataaacgtgaaatgtctttggatttgggaatcttataagttctgtatgagaccacagatctgaagttcgagggcgacacccggtagacaagacttcactattcaagagatagtgaagtcttgtctaccttttttagatctggatccaagcttccatggaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttgggtttatatatcttgtggaaaggacgaaacaccgctgaagggcatcgacttcagatgtcttctgtcaagttattcaagagataacttgacagaagacatcttttttcatatgccatgggcggccgctctagactgcagtatttagcatgccccacccatctgcaaggcattctggatagtgtcaaaacagccggaaatcaagtccgtttatctcaaactttagcattttgggaataaatgatatttgctatgctggttaaattagattttagttaaatttcctgctgaagctctagtacgataagtaacttgacctaagtgtaaagttgagatttccttcaggtttatatagcttgtgcgccgcctgggtacctccaactacaagacccgcgccggtgaactcgaagaagatatttcaagagaatatcttcttcgagttcacttttttggtacctctagactcgagaactagt

SEQ ID NO: 61 - DNA, 3684 bp, Artificial Sequence
  Expression construct (5′ to 3′) used in Merienne, 2017
gcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatacgcgtaccgctagccaggaagagtttgtagaaacgcaaaaaggccatccgtcaggatggccttctgcttagtttgatgcctggcagtttatggcgggcgtcctgcccgccaccctccgggccgttgcttcacaacgttcaaatccgctcccggcggatttgtcctactcaggagagcgttcaccgacaaacaacagataaaacgaaaggcccagtcttccgactgagcctttcgttttatttgatgcctggcagttccctactctcgcgttaacgctagcatggatgttttcccagtcacgacgttgtaaaacgacggccagtcttaagctcgggccccaaataatgattttattttgactgatagtgacctgttcgttgcaacaaattgatgagcaatgcttttttataatgccaactttgtacaaaaaagcaggcttcatcgatcaattgggatccaattcgaacgctgacgtcatcaacccgctccaaggaatcgcgggcccagtgtcactaggcgggaacacccagcgcgcgtgcgccctggcaggaagatggctgtgagggacaggggagtggcgccctgcaatatttgcatgtcgctatgtgttctgggaaatcaccataaacgtgaaatgtctttggatttgggaatcttataagttctgtatgagaccacagatctgaagttcgagggcgacacccgttttagagctagaaatagcaagttaaaataaggctagtccgttatcaacttgaaaaagtggcaccgagtcggtgcttttttagatctggatccaagcttccatggaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttgggtttatatatcttgtggaaaggacgaaacaccgctgaagggcatcgacttcagttttagagctagaaatagcaagttaaaataaggctagtccgttatcaacttgaaaaagtggcaccgagtcggtgcttttttcatatgccatgggcggccgctctagactgcagtatttagcatgccccacccatctgcaaggcattctggatagtgtcaaaacagccggaaatcaagtccgtttatctcaaactttagcattttgggaataaatgatatttgctatgctggttaaattagattttagttaaatttcctgctgaagctctagtacgataagtaacttgacctaagtgtaaagttgagatttccttcaggtttatatagcttgtgcgccgcctgggtacctccaactacaagacccgcgccggttttagagctagaaatagcaagttaaaataaggctagtccgttatcaacttgaaaaagtggcaccgagtcggtgcttttttggtacctctagactcgagactagtaggcctgacccagctttcttgtacaaagttggcattataagaaagcattgcttatcaatttgttgcaacgaacaggtcactatcagtcaaaataaaatcattatttgccatccagctgatatcccctatagtgagtcgtattacatggtcatagctgtttcctggcagctctggcccgtgtctcaaaatctctgatgttacattgcacaagataaaaatatatcatcatgaacaataaaactgtctgcttacataaacagtaatacaaggggtgttatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcgcaagctcatgaccaaaatcccttaacgtgagttacgcgtcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaa